(19) United States
(12) Patent Application Publication
(10) Pub. No.: US 2007/0161081 A1
(43) Pub. Date: Jul. 12, 2007

(54) Hepatocyte growth factor intron fusion proteins

(75) Inventors: Pei Jin, Palo Alto, CA (US); H. Michael Shepard, San Francisco, CA (US); Irene Y. Ni, Foster City, CA (US)

Correspondence Address: MORRISON & FOERSTER LLP, 755 PAGE MILL RD, PALO ALTO, CA, 94304-1018, US

(73) Assignee: Receptor Biologix, Inc., South San Francisco, CA (US)

(21) Appl. No.: 11/590,955

(22) Filed: Oct. 31, 2006

Related U.S. Application Data
(60) Provisional application No. 60/735,609, filed on Nov. 10, 2005.

(51) Int. Cl. A61K 38/18; C07K 14/475; C07H 21/04; C12P 21/06

(52) U.S. Cl. 435/69.1; 435/320.1; 435/325; 514/12; 530/399; 536/23.5

(57) ABSTRACT

Isoforms of ligands, including isoforms of hepatocyte growth factor (HGF) containing an intron-encoded portion, and pharmaceutical compositions containing HGF isoforms are provided. The HGF ligand isoforms and compositions containing them can be used in methods of treatment of diseases, such as cancer and other angiogenic diseases.

### RELATED APPLICATIONS

Benefit of priority is claimed to U.S. provisional application Ser. No. 60/735,609, filed Nov. 10, 2005, entitled “HEPATOCYTE GROWTH FACTOR INTRON FUSION PROTEINS,” to Pei Jin, H. Michael Shepard, and Irene Ni.

This application is related to International PCT Application Serial No. (Attorney Docket No. 17118-045W01/2824PC), filed the same day herewith, entitled “HEPATOCYTE GROWTH FACTOR INTRON FUSION PROTEINS,” to Receptor Biologix, Inc., Pei Jin, H. Michael Shepard, and Irene Ni, which also claims priority to U.S. Provisional Application Ser. No. 60/735,609.

This application also is related to U.S. application Ser. No. 10/846,113, filed May 14, 2004, and to corresponding International PCT application No. WO 05/016966, published Feb. 24, 2005, entitled “INTRON FUSION PROTEINS, AND METHODS OF IDENTIFYING AND USING SAME.” This application also is related to U.S. application Ser. No. 11/129,740, filed May 13, 2005, and to corresponding International PCT application No. PCT/US2005/17051, filed May 13, 2005, entitled “CELL SURFACE RECEPTOR ISOFORMS AND METHODS OF IDENTIFYING AND USING THE SAME.” The application also is related to U.S. application Ser. No. 11/429,090 and to corresponding International PCT application No. PCT/US2006/17786, which each claim priority to U.S. provisional application No. 60/678,076, entitled “ISOFORMS OF RECEPTOR FOR ADVANCED GLYCATION END PRODUCTS (RAGE) AND METHODS OF IDENTIFYING AND USING SAME”, filed May 4, 2005. This application also is related to U.S. application No. (Attorney Docket No. 17118-041001/ 2822) and to International application No. (Attorney Docket No. 17118-041WO1/ 2822PC) filed the same day herewith, which each claim priority to provisional application No. 60/736,134, entitled “METHODS FOR PRODUCTION OF RECEPTOR AND LIGAND ISOFORMS,” filed Nov. 10, 2005.

The subject matter of each of the above-noted applications, provisional applications and international applications as well as any applications noted throughout the disclosure herein is incorporated herein by reference thereto.

### INCORPORATION BY REFERENCE OF SEQUENCE LISTING PROVIDED ON COMPACT DISCS

An electronic version on compact disc (CD) ROM of the Sequence Listing is filed herewith in duplicate, the contents of which are incorporated by reference in their entirety. The computer-readable file on each of the aforementioned duplicate compact discs created on Oct. 31, 2006, is identical, 971 kilobytes in size, and is entitled 2824SEQ.001.txt.

### FIELD OF THE INVENTION

Isoforms of ligands, including isoforms of hepatocyte growth factor (HGF) containing an intron-encoded portion, and pharmaceutical compositions containing HGF isoforms are provided. The HGF ligand isoforms and compositions containing them can be used in methods of treatment of diseases, such as cancer and other angiogenic diseases.

### BACKGROUND

Growth factors are produced by many different cell types and exert their effects via autocrine and paracrine mechanisms. They function as stimulators or inhibitors of the division, differentiation and migration of cells and are involved in carcinogenesis, in which they influence a variety of functions including cell proliferation, cell invasion, metastasis formation, angiogenesis, local immune system functions and extracellular matrix synthesis. In particular, invasion of tumor cells and subsequent establishment of metastasis are devastating events associated with cancer progression and severity.

Hepatocyte growth factor (HGF, also called scatter factor) is a growth factor ligand for the c-met protooncogene (MET receptor). In normal tissues, HGF plays a role in the construction and reconstruction of tissues during organogenesis and tissue regeneration including the development of embryonic tissues including the liver, kidney, lung, mammary gland, teeth, placenta, and skeletal muscle. HGF also plays a role in the regeneration and protection of mature tissues. In malignant tissues, however, tumor cells utilize the biological actions of HGF for their invasion and metastatic behavior. HGF promotes the invasive behavior of tumors by regulating cell-cell adhesion, cell-matrix association, proteolytic breakdown of the extracellular matrix, cellular locomotion, and angiogenesis.

Because of its involvement in proliferative and angiogenic diseases, including many cancers, HGF is a target for therapeutic intervention. Small molecule therapeutics that target the HGF or its receptor, MET, have been designed. While it may be possible to design small molecules as therapeutics that target such cell surface receptors and/or other angiogenic receptors or their ligands, there are, however, a number of limitations with such strategies. Small molecules can be promiscuous and affect receptors other than the intended target. Additionally, some small molecules bind irreversibly or substantially irreversibly to the receptors (i.e. subnanomolar binding affinity). The merits of such approaches have not been validated. Antibodies against receptor and/or receptor ligands can be used as therapeutics. Antibody treatments, however, can result in an immune response in a subject and thus, such treatments often need extensive tailoring to avoid complications in treatment. Thus, there exists an unmet need for therapeutics for treatment of diseases, including cancers and other diseases involving undesirable cell proliferation and angiogenic reactions. Accordingly, among the objects herein, it is an object to provide such therapeutics and methods for identifying or discovering candidate therapeutics and methods of treatment.

### SUMMARY

Provided herein are therapeutics for treatment of diseases, including cancers and other diseases involving undesirable cell proliferation, angiogenic and inflammatory reactions. Also provided are methods for identifying or discovering candidate therapeutics and methods of treatment using the therapeutics. The therapeutics are polypeptides or modified polypeptides, such as polypeptides including peptidomimetic bonds.

HGF polypeptide isoforms are provided. Among the isoforms are isolated HGF polypeptide isoforms that contain all or a portion of a K4 domain of an HGF ligand. The portion is sufficient to confer an activity exhibited by the K4 domain, or contains at least 2, 3, 4, 5, 6, 7, 8, 9, 10, 15, 20, 25, 30 or more amino acids therefrom. Among these are HGF isoform polypeptides that are intron fusion proteins. The isolated HGF polypeptide isoforms also can include all or part of a SerP domain.

Exemplary of the HGF isoforms are those encoded by a sequence of nucleotides that includes all or a portion of an intron selected from among introns 1, 2, 3, 4, 5, 6, 7, 8, 9, 10, 11, 12, 13, 14, 15, 16, and 17 of a cognate HGF gene (i.e., the gene that encodes the HGF ligand that includes all exons), such as the human HGF gene. The sequence of an allele thereof is set forth in SEQ ID NO: 1. Also provided are HGF isoforms that are allelic, species or other variants thereof, including, for example, isoforms for which the cognate HGF ligand has at least 80%, 85%, 90%, 95%, 96%, 97%, 98% or 99% or more sequence identity with the sequence of amino acids encoded by the corresponding portions of SEQ ID NO: 1. The portion encoded by an intron can be one codon, including a stop codon, or more codons, so that the resulting HGF isoforms either stops at the end of the exon or includes 1, 2, 3, or more amino acids encoded by an intron.

Also provided are isolated HGF polypeptide isoforms described above that include all or part of an N-terminal domain, all or part of a K1 domain, all or part of a K2 domain, or all or part of a K3 domain or any combination thereof. Among the isoforms provided are those that are encoded by a nucleic acid molecule that includes all or a portion of intron 11. The portion can be one codon, including a stop codon, so that the resulting isoforms stops at the end of the exon and includes no other amino acids, or the portion can be more than one codon so that the isoform includes, 1, 2, 3, 4, 5, 6, 7, 8, 9, 10, 11, 12, 13, 14, 15, 16, 17 18, 19, 20, 21 up to all of the amino acids encoded by an intron. In exemplary embodiments the isoforms includes one, two or three amino acids encoded by intron 11.

Provided are HGF polypeptide isoforms that have at least 80%, 85%, 90%, 95%, 96%, 97%, 98%, 99% or 100% sequence identity with a sequence of amino acids set forth in any of SEQ ID NOS: 10, 12, 18, or 20 and/or is an allelic or species variant thereof. An exemplary allelic variant of the HGF polypeptide has the sequence of amino acids set forth in SEQ ID NO: 16. As a result, HGF isoforms will include the variations present in any exon or intron that is part of the particular isoforms. Among HGF isoform variants provided herein are those that contain the same number of amino acids as set forth in any of SEQ ID NOS: 10, 12, 18, or 20.

Among the isoforms provided are those that are encoded by a nucleic acid molecule that includes all or a portion of intron 13. The portion can be one codon, including a stop codon, so that the resulting isoforms stop at the end of the exon and include no other amino acids, or the portion can be more than one codon so that the isoforms includes, 1, 2, 3, 4, 5, 6, 7, 8, 9, 10, 11, 12, 13, 14, 15, 16, 17 18, 19, 20, 21 up to all of the amino acids encoded by intron 13. In exemplary embodiments the isoforms include one, two or three amino acids encoded by intron 13. Provided are HGF polypeptide isoforms that have at least 80%, 85%, 90%, 95%, 96%, 97%, 98%, 99% or 100% sequence identity with a sequence of amino acids set forth in SEQ ID NO: 14, as well as allelic and species variants thereof, including portions of the allelic variant whose sequence is set forth in SEQ ID NO: 16. This includes variants that contain the same number of amino acids as set forth in SEQ ID NO: 14.

The isolated HGF polypeptide isoforms provided herein include those that act as an antagonist of an HGF polypeptide, such as the cognate HGF polypeptide; those that bind to a MET receptor; those that inhibit one or more MET-mediated activities selected from among mitogenesis, morphogenesis and motogenesis; those that inhibit angiogenesis; those that bind to a glycosaminoglycan, such as heparin sulfate; those that bind to an angiogenic molecule, such as any of ATP synthase, angiomotin, αvβ3 integrin, annexin II, MET, VEGFR, and FGFR; those that inhibit angiogenesis induced by a cognate HGF, FGF-2 and/or VEGF; and those that are an HGF antagonist and inhibit angiogenesis. An HGF isoform can possess one or more of any of these activities and/or other activities.

Also provided are pharmaceutical compositions that contain one or more of the HGF polypeptide isoforms in a pharmaceutically acceptable carrier. The pharmaceutical compositions can be formulated for administration by any suitable route. The compositions can include additional active agents, including, but not limited to, other anti-cancer and/or anti-angiogenesis agents. The amount of HGF isoforms is effective for a particular activity, including antagonizing a cognate HGF polypeptide, such as where antagonizing a cognate HGF inhibits one or more of a MET-mediated activity selected from any one or more of mitogenesis, motogenesis and morphogenesis; and/or inhibiting angiogenesis, such as angiogenesis induced by a cognate HGF, FGF-2 and/or VEGF.

Nucleic acid molecules encoding any of the HGF polypeptides are provided. Nucleic acid molecules provided contain all or part of an exon and at least one codon from an intron other than intron 5. The intron can contain a stop codon at any locus including the first locus. For example, provided are nucleic acid molecules that encode an open reading frame that spans an exon intron junction, where the open reading frame terminates at the stop codon in an intron (other than in intron 5), such as intron 11 or 13. Also provided are nucleic acid molecules where a stop codon is the first codon in the intron, such as intron 13. In exemplary embodiments provided are nucleic acid molecules containing a sequence of nucleotides set forth in any one of SEQ ID NOS: 9, 11, 13, 17, or 19, or an allelic variant thereof, such one or more of the variations in SEQ ID NO:15, or species variants. Also provided are nucleic acid molecules that encode an isoform, as noted above, that includes all or part of a K4 domain and that has at least 60, 65, 70, 75, 80, 85, 90, 91, 92, 93, 94, 95, 96, 97, 98, 99% sequence identity to any of SEQ ID NOS: 9, 11, 13, 17, or 19; or that hybridizes under conditions of medium or high stringency along at least 70% of its full length to a nucleic acid molecule comprising a sequence of nucleotides set forth in any of SEQ ID NOS: 9, 11, 13, 17, or 19 wherein the encoded polypeptide contains a K4 domain and contains at least one codon from an intron. Also provide are nucleic acid molecules that contain degenerate codons of any of the noted nucleic acid molecules. Also provided are nucleic acid molecules that are splice variants of an HGF gene and that include all or a portion of an intron other than intron 5. Polypeptides encoded by any of these nucleic acid molecules are provided, as are vectors that contain any of the nucleic acid molecules. The vectors include eukaryotic and prokaryotic vectors, expression vectors, such as mammalian expression vectors, and vectors suitable for gene therapy. Exemplary vectors are viral vectors including, but not limited to adenovirus vectors, adeno-associated virus vectors, EBV vectors, SV40 vectors, cytomegalovirus vectors, vaccinia virus vectors, herpesvirus vectors, retroviral vectors and lentivirus vectors. Also included are artificial chromosomes. Vectors can be episomal or integrative.

Cells containing the vectors are provided. The cells include eukaryotic cells, including mammalian and insect cells, and prokaryotic cells.

Methods of treatment are provided. The methods can be effected by administering a pharmaceutical composition containing an HGF isoform provided herein, and/or by gene therapy through introduction of a nucleic acid molecule encoding such isoforms. Gene therapy methods include ex vivo methods, which includes introduction into host cells removed from the subject or a compatible source, and in vivo methods, which include topical, local and system administration. Ex vivo treatment can include administering the nucleic acid into a cell in vitro, followed by administration of the cell into the subject. The cell can be from a suitable donor or from the subject, such as a human, to be treated.

Methods for treating a disease or condition by administering a pharmaceutical composition containing one or more isoforms are provided. The isoforms can be one that inhibits angiogenesis, cell proliferation, cell migration, tumor cell growth and/or tumor cell metastasis. Conditions treated include, but are not limited to, cancer, angiogenic disease and malaria. Angiogenic diseases include ocular disease, endometriosis, arthritis and other chronic or acute inflammatory diseases. Exemplary diseases are rheumatoid arthritis, osteoarthritis, psoriasis, Osler-Webber syndrome, endometriosis, Still's disease, angiogenesis of the heart-muscle, peripheral hemangiectasis, hemophilic arthritis, age-related macular degeneration, retinopathy of prematurity, rejection to keratoplasty, systemic lupus erythematosus, atherosclerosis, neovascular glaucoma, choroidal neovascularization, retrolental fibroplasias, perosis, neurofibroma, hemangioma, acoustic neuroma, neurofibroma, trachoma, suppurative granuloma, and diabetes related diseases, such as proliferative diabetic retinopathy and vascular diseases, inflammatory lung disease, Crohn's disease and psoriasis. Cancers that can be treated include gastric, lung, breast, colon, pancreatic, prostate and other tumors and blood cancer, and include carcinomas, lymphomas, blastomas, sarcoma, and leukemia or lymphoid malignancies, squamous cell cancers, lung cancers, small-cell lung cancers, non-small cell lung cancers, adenocarcinomas of the lung, squamous carcinomas of the lung, cancers of the peritoneum, hepatocellular cancers, gastric or stomach cancers, gastrointestinal cancers, pancreatic cancers, glioblastomas, cervical cancers, ovarian cancers, liver cancers, bladder cancers, hepatomas, breast cancers, colon cancers, rectal cancers, colorectal cancers, endometrial or uterine carcinomas, salivary gland carcinomas, kidney or renal cancers, prostate cancers, vulval cancers, thyroid cancers, hepatic carcinomas, anal carcinomas, penile carcinomas and head and neck cancers. The particular isoforms to employ can be determined empirically as needed. The compositions can be administered to inhibit tumor invasion or metastasis of a tumor and/or to inhibit angiogenesis.

Conjugates that contain HGF isoforms linked, directly or indirectly via a linker to another moiety are provided. Conjugates include fusion proteins and also chemical conjugates. Conjugates can contain HGF isoforms or domains thereof or functional portion thereof, and a second portion from a different HGF isoform or from a cell surface receptor (CSR) isoform or ligand isoform. Cell surface receptor isoforms include, for example, all or part of an extracellular domain of the cell surface receptor isoforms. Cell surface receptor isoforms include receptor tyrosine kinases, such as all or part of a herstatin polypeptide. Exemplary herstatin polypeptides include a sequence of amino acids set forth in any one of SEQ ID NOS:186-200 or allelic or species variants thereof.

Also provided are conjugates that are chimeric polypeptides that contain all or at least one domain of an HGF isoform and all of or at least one domain of a different HGF isoform or of another cell surface receptor isoform, such as an intron fusion protein. Other chimeric polypeptides include all of or at least one domain of an HGF isoform and an intron-encoded portion of a cell surface receptor isoform.

Also provided are combinations that contain one or more HGF isoform(s) and a containing one or more other cell surface receptor isoforms and/or a therapeutic drug. Such combinations include those where the isoforms and/or drugs are in separate compositions or in a single composition. The combinations can be provided as a kit, with optional instructions for use and/or with other reagents and utensils and components for administration and use of the components of the combination. Methods of treatment by administering the combinations are provided. Each component can be administered separately, simultaneously, intermittently, in a single composition or combinations thereof.

Cell surface receptor isoforms for inclusion in the combinations or conjugates include, but are not limited to, isoforms of VEGFR, FGFR, DDR, TNFR, PDGFR, MET, TIE, RAGE, EPH or HER. The isoforms can be intron fusion proteins. Exemplary Met isoforms contain a sequence of amino acids selected from any one of SEQ ID NOS: 85, 87, 89, 91, 93, 95, 97, 99, 101, 103, 105, 107, 109, 111, 113 and 114.

Also provided are fusion proteins or conjugates that contain a fragment of a CD45 polypeptide linked directly or via a linker to a protein, where the fragment of CD45 is selected to add carbohydrates or glycosylation sites, and hence includes at least one such site and a sufficient amount to extend serum half-life of a linked moiety, such as a polypeptides. Fusion proteins contain the CD45 polypeptide or fragment thereof linked directly or indirectly to polypeptide; conjugates contain the CD45 polypeptide or fragment thereof linked directly or indirectly to a non-peptide moiety, typically a therapeutic agent. Linked agents include proteins and other agents, such as small molecule therapeutics. Linked proteins include therapeutic proteins, such as a CSR or ligand isoform or, a cytokine, CSR, ligand, growth factor, hormone or forms thereof that include additional amino acids on the end. The CD45 polypeptide or fragment contains a sufficient number of glycosylation sites or carbohydrates, whereby serum half-life of the protein is increased by 1%, 5%, 10%, 20%, 30%, 40%, 50%, 60%, 70%, 80%, 90%, 95%, 96%, 97%, 98%, 99% or greater. Linkage can be direct or via a chemical linkage, such as a peptide linkage or a chemical linker, including a linkage resulting from a heterobifunctional linker, and/or it can be a photocleavable linker. The conjugate or fusion protein optionally includes a polypeptide or peptide or amino acid linker, which can contain 1-30, 1-10, 2-10 or 2-15 or more amino acid residues. An exemplary CD45 polypeptide or fragment thereof contains the sequence of amino acids set forth in any of SEQ ID NOS: 272, 274, 275, 276, 277, 278, 279, 281, 283, 285, 287, 289, 291, 293 and 295, or fragments thereof or variants thereof.

The protein linked to the CD45 protein can be a ligand, such as HGF or isoforms hereof, or a CSR or CSR isoform or a form of ligand, CSR or isoform containing additional amino acids. Exemplary proteins include, but are not limited to, those that contain a sequence of amino acids set forth in any of SEQ ID NOS: 3, 10, 12, 14, 16, 18, 20, 22, 24, 26, 28, 30, 32, 34, 35, 37, 39, 40, 42, 44, 46, 47, 49, 50, 52, 54, 56, 58, 59, 60, 61, 62, 63, 64, 65, 67, 69, 71, 73, 75, 77, 78, 80, 82, 83, 85, 87, 89, 91, 93, 95, 97, 99, 101, 103, 105, 107, 109, 111, 113, 114, 116, 118, 120, 122, 124, 126, 127, 128, 130, 132, 134, 136, 138, 140, 142, 144, 145, 146, 147, 148, 150, 152, 154, 156, 158, 160, 161, 162, 163, 164, 165, 166, 167, 169, 171, 172, 173, 174, 175, 176, 177, 179, 181, 183, 185, 186, 187, 188, 189, 190, 191, 192, 193, 194, 195, 196, 197, 198, 199, 200, 216, 217, 218, 219, 220, 221, 222, 223, 224, 225, 226, 227, 228, 229, 230, 246, 247, 248, 249, 250 and 251, or allelic variants thereof.

### BRIEF DESCRIPTION OF THE FIGURES

FIG. 1 depicts the genomic organization of an exemplary HGF gene (see SEQ ID NO: 1 for the sequence thereof). The HGF gene contains 18 exons (solid) interrupted by 17 introns (dashed). A predominant splice form of HGF contains a polypeptide encoded by the 18 exons. HGF isoforms provided herein are encoded by alternatively spliced variants of the HGF gene and are encoded by exons and at least one codon from an intron portion. The exon-intron organization of nucleic acid encoding exemplary HGF isoforms SR023A01, SR023A08, and SR023E09 is depicted. The asterix depicts a stop codon within the intron portion of the gene thereby resulting in a truncated isoform. The open box in exon 5 of the SR023A02 isoform denotes a deleted portion of exon 5. HGF isoforms include all or part of any one or more of introns of the HGF gene operatively linked to an exon of HGF resulting in an intron fusion protein of HGF.

FIG. 2 depicts the domain organization of a cognate HGF. The figure depicts the domain organization of HGF isoforms including SR023A02, SR023A08, and SR023E09.

FIG. 3 depicts an overview of the contribution of HGF in cancer progression, including tumor growth and angiogenesis. HGF acts (A) as a morphogenic and mitogenic factor promoting the scattering and migration, invasion, and metastasis of cancer cells, (B) as a mitogenic factor stimulating the proliferation of cancer cells thereby promoting tumor growth, and (C) as an angiogenic factor thereby promoting angiogenesis and growth of blood vessels which contributes to the metastasis and growth of primary and secondary tumors. Target points for modulation of these pathways by HGF isoforms are indicated.

### DETAILED DESCRIPTION

Outline

A. DEFINITIONS

B. HEPATOCYTE GROWTH FACTOR (HGF) AND MET RECEPTOR
1. HGF
   a. HGF DOMAIN STRUCTURE
      i. N TERMINAL DOMAIN
      ii. KRINGLE DOMAINS
      iii. β-CHAIN
2. HGF VARIANTS
   a. HGF SPLICE VARIANTS
   b. HGF ALLELIC VARIANTS
3. MET RECEPTOR

C. HGF ISOFORMS
1. CLASSES OF HGF ISOFORMS
2. ALTERNATIVE SPLICING AND GENERATION OF HGF ISOFORMS
   a. INTRON MODIFICATION AND INTRON FUSION PROTEINS
      i. NATURAL INTRON FUSION PROTEINS
      ii. COMBINATORIAL INTRON FUSION PROTEINS
   b. ISOFORMS GENERATED BY EXON MODIFICATIONS
2. HGF ISOFORM POLYPEPTIDE STRUCTURE
3. HGF ISOFORM ACTIVITIES
   a. CELL SURFACE ACTION ALTERATIONS
   b. COMPETITIVE ANTAGONIST
   c. NEGATIVELY ACTING AND INHIBITORY ISOFORMS

D. METHODS FOR IDENTIFYING AND GENERATING HGF ISOFORMS
1. METHODS FOR IDENTIFYING AND ISOLATING ISOFORMS
2. IDENTIFICATION OF ALLELIC AND SPECIES VARIANTS OF ISOFORMS

E. EXEMPLARY HGF ISOFORMS
1. HGF ISOFORMS
2. HGF INTRON FUSION PROTEINS

F. METHODS FOR PRODUCING NUCLEIC ACIDS ENCODING HGF ISOFORM POLYPEPTIDES
1. SYNTHETIC GENES AND POLYPEPTIDES
2. METHODS OF CLONING AND ISOLATING HGF ISOFORMS
3. EXPRESSION SYSTEMS
   a. PROKARYOTIC EXPRESSION
   b. YEAST
   c. INSECT CELLS
   d. MAMMALIAN CELLS
   e. PLANTS

G. ISOFORM CONJUGATES
1. ISOFORM FUSIONS
   a. HGF ISOFORM FUSIONS FOR IMPROVED PRODUCTION OF HGF ISOFORM POLYPEPTIDES
      i. TISSUE PLASMINOGEN ACTIVATOR
      ii. TPA-HGF INTRON FUSION PROTEIN FUSIONS
   b. CHIMERIC AND SYNTHETIC INTRON FUSION POLYPEPTIDES
   c. HGF MULTIMERS AND MULTIMERIZATION DOMAINS
      i. PEPTIDE LINKERS
      ii. POLYPEPTIDE MULTIMERIZATION DOMAINS
         (a) IMMUNOGLOBULIN DOMAIN
         (i) FC DOMAIN
         (ii) PROTUBERANCES-INTO-CAVITY (I.E. KNOBS AND HOLES)
         (b) LEUCINE ZIPPERS
         (i) FOS AND JUN
         (ii) GCN4
         (c) OTHER MULTIMERIZATION DOMAINS R/PKA-AD/AKAP
   d. METHODS OF GENERATING AND CLONING HGF FUSIONS
2. TARGETING AGENT/TARGETING AGENT CONJUGATES
3. PEPTIDOMIMETIC ISOFORMS

H. METHODS FOR ALTERING SERUM HALF-LIFE AND OTHER THERAPEUTIC PROPERTIES
1. N-LINKED AND O-LINKED GLYCOSYLATION
2. EFFECTS OF GLYCOSYLATION
3. THERAPEUTIC USES FOR GLYCOSYLATION
4. USE OF CD45 FOR ALTERING SERUM HALF-LIFE
   a. CD45 FUNCTION
   b. CD45 DIMERIZATION AND GLYCOSYLATION
   c. CD45 FUSION PROTEINS
   d. CONJUGATES OF CD45 FUSION PROTEINS
   e. THERAPEUTIC CD45 FUSION PROTEINS
   f. METHODS FOR MEASURING GLYCOSYLATION
   g. METHODS OF PRODUCTION AND INCREASING GLYCOSYLATION
   h. HGF-CD45 FUSION PROTEINS AND THERAPEUTIC USES

I. METHODS OF PREPARING AND ISOLATING HGF ISOFORM-SPECIFIC ANTIBODIES

J. ASSAYS TO ASSESS OR MONITOR HGF ISOFORM ACTIVITIES
1. LIGAND BINDING ASSAYS AND HGF BINDING ASSAYS
2. LIGAND DIMERIZATION
3. COMPLEXATION
4. MET AND ERK1/2 PHOSPHORYLATION ASSAYS
5. MORPHOGENIC/ANGIOGENIC ASSAYS
6. MITOGENIC/PROLIFERATION ASSAYS
7. MOTOGENIC/CELL MIGRATION ASSAYS
8. APOPTOTIC ASSAYS
9. ANIMAL MODELS
   a. TUMOR SUPPRESSION ASSAYS
   b. ANGIOGENIC DISEASE

K. PREPARATION, FORMULATION AND ADMINISTRATION OF HGF ISOFORMS AND HGF ISOFORM COMPOSITIONS

L. IN VIVO EXPRESSION OF HGF ISOFORMS AND GENE THERAPY
1. DELIVERY OF HGF
   a. VECTORS—EPISOMAL AND INTEGRATING
   b. ARTIFICIAL CHROMOSOMES AND OTHER NON-VIRAL VECTOR DELIVERY METHODS
   c. LIPOSOMES AND OTHER ENCAPSULATED FORMS AND ADMINISTRATION OF CELLS CONTAINING THE NUCLEIC ACID MOLECULES
2. IN VITRO AND EX VIVO DELIVERY
3. SYSTEMIC, LOCAL AND TOPICAL DELIVERY

M. HGF AND CANCER AND ANGIOGENESIS
1. TUMOR GROWTH AND METASTASIS
   a. MITOGENESIS
   b. MOTOGENESIS AND MORPHOGENESIS
2. ANGIOGENESIS
   a. THE ANGIOGENIC PROCESS
   b. CELL SURFACE RECEPTORS IN ANGIOGENESIS
   c. HGF IN TUMOR ANGIOGENESIS
   d. HGF IN OTHER VASCULAR DISEASES
3. HGF ISOFORMS AND CANCER AND ANGIOGENESIS

N. EXEMPLARY TREATMENTS WITH HGF ISOFORMS
1. CANCER
2. ANGIOGENIC DISEASES
   a. ARTHRITIS AND CHRONIC INFLAMMATORY DISEASES
   b. OCULAR DISEASES
   c. ENDOMETRIOSIS
3. MALARIA
4. COMBINATION THERAPIES
5. EVALUATION OF HGF ISOFORM ACTIVITIES

O. EXAMPLES

### A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a ligand is an extracellular substance, generally a polypeptide, that binds to one or more receptors. A ligand can be soluble or can be a transmembrane protein. For purposes herein, a ligand binds to a receptor and induces signal transduction by the receptor.

As used herein, signal transduction refers to a series of sequential events, such as protein phosphorylations, consequent upon binding of ligand by a transmembrane cell surface receptor, that transfer a signal through a series of intermediate molecules until final regulatory molecules, such as transcription factors, are modified in response to the signal. Responses triggered by signal transduction include the activation of specific genes. Gene activation leads to further effects, since genes are expressed as proteins, many of which are enzymes, transcription factors, or other regulators of metabolic activity that mediate any one or more biological activities of a ligand-receptor interaction.

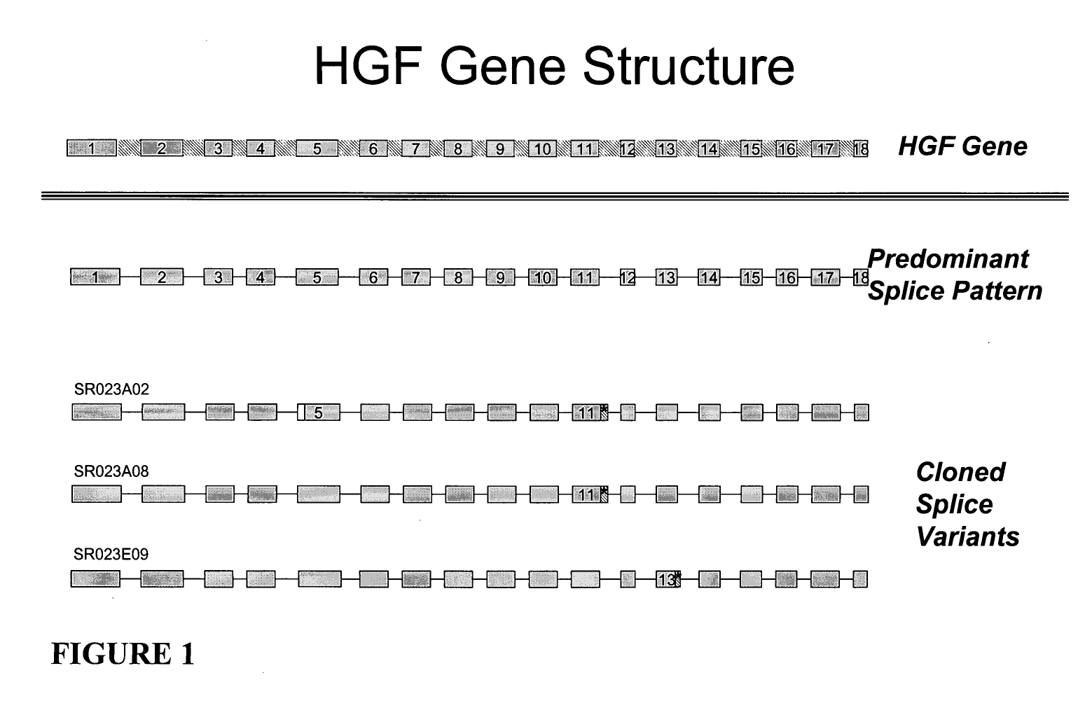

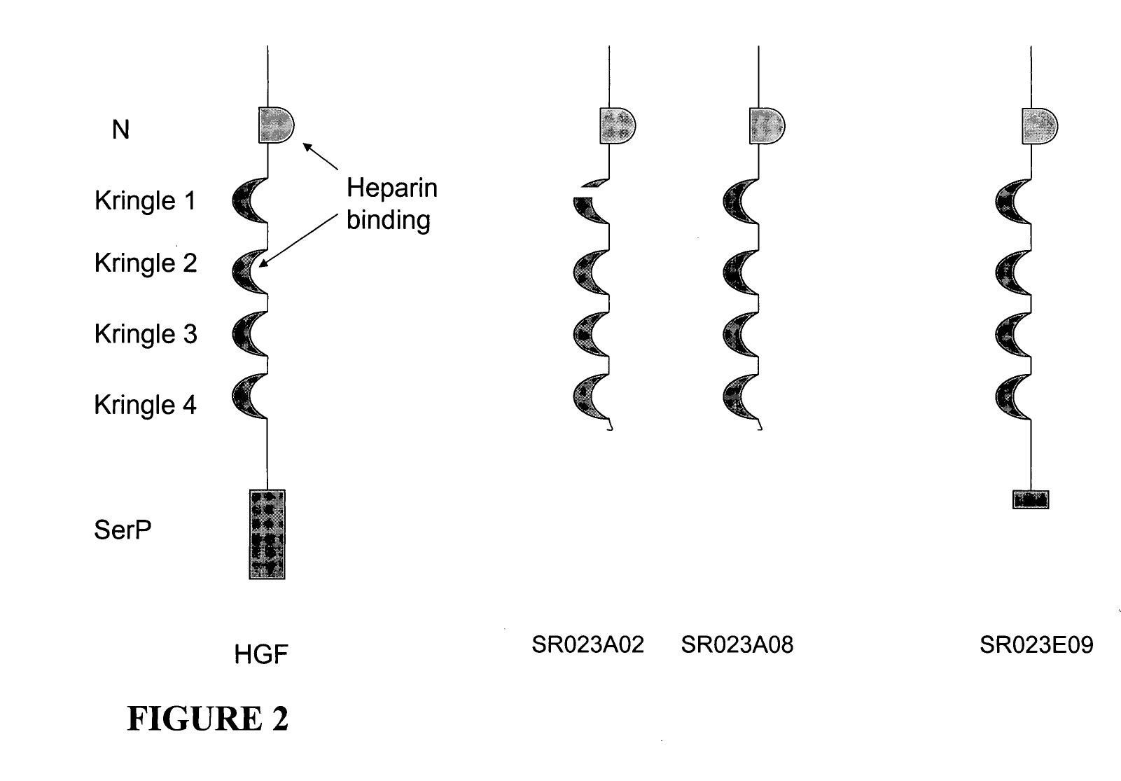

As used herein, hepatocyte growth factor (HGF) refers to a ligand of the MET receptor that induces mitogenesis, morphogenesis, and motogenesis. Normally, HGF is involved in organogenesis and tissue regeneration in developing and mature tissues. In malignant tissues, HGF contributes to cancer progression by promoting the invasion, migration, and proliferation of tumor cells, thereby contributing to tumorigenesis. HGF also is an angiogenic factor contributing to cancer growth and spread, and other angiogenic diseases. As an example, a human HGF encodes a 728 amino acid residue ligand with a 31 amino acid signal peptide, an N-terminal domain between amino acid 34-124, a Kringle 1 domain between amino acids 128-206, a Kringle 2 domain between amino acids 241-288, a Kringle 3 domain between amino acids 305-383, a Kringle 4 domain between amino acids 391-469, and a serine protease domain between amino acids 495-728 (see e.g., FIG. 2, SEQ ID NO:3). The precursor protein is a monomer which is cleaved to generate a disulfide-linked heterodimer composed of a 69 kDa α-chain and a 34 kDa β-chain. The HGF gene is composed of 18 exons interrupted by 17 introns (see e.g., FIG. 1). An exemplary genomic sequence of HGF is set forth as SEQ ID NO:1. Alternative splice variants of HGF exist. Two known splice variants, NK1 and NK2, are truncated HGF isoforms that contain an N-terminal domain, and a Kringle 1 domain (NK1) or a Kringle 1 and a Kringle 2 domain (NK2). NK1 and NK2 are partial agonists of MET signaling. An engineered variant of HGF, termed NK4, has been generated by enzymatic cleavage of HGF and is an antagonist of HGF-MET signaling. HGF includes allelic variants of HGF including species variants and any one of the allelic variations of HGF set forth in SEQ ID NO:1. HGF is also found in different species besides human, including cow, dog, cat, mouse, rat, horse, or others. Exemplary species variants of HGF are set forth in any one of SEQ ID NOS: 246-251.

As used herein, a cell surface receptor (CSR) is a protein that is expressed on the surface of a cell and typically includes a transmembrane domain or other moiety that anchors it to the surface of a cell. As a receptor it binds to ligands that mediate or participate in an activity of the cell surface receptor, such as signal transduction or ligand internalization. Cell surface receptors include, but are not limited to, single transmembrane receptors and G-protein coupled receptors. Receptor tyrosine kinases, such as growth factor receptors, also are among such cell surface receptors.

As used herein, a receptor tyrosine kinase (RTK) refers to a protein, typically a glycoprotein, that is a member of the growth factor receptor family of proteins. Growth factor receptors are typically involved in cellular processes including cell growth, cell division, differentiation, metabolism and cell migration. RTKs also are known to be involved in cell proliferation, differentiation and determination of cell fate as well as tumor growth. RTKs have a conserved domain structure including an extracellular domain, a membrane-spanning (transmembrane) domain and an intracellular tyrosine kinase domain. Typically, the extracellular domain binds to a polypeptide growth factor or a cell membrane-associated molecule or other ligand. The tyrosine kinase domain is involved in positive and negative regulation of the receptor. An exemplary RTK is the MET receptor.

Dimerization of RTKs activates the catalytic tyrosine kinase domain of the receptor and tyrosine autophosphorylation. Autophosphorylation in the kinase domain maintains the tyrosine kinase domain in an activated state. Autophosphorylation in other regions of the protein influences interactions of the receptor with other cellular proteins. In some RTKs, ligand binding to the extracellular domain leads to dimerization of the receptor. In some RTKs, the receptor can dimerize in the absence of ligand. Dimerization also can be increased by receptor overexpression.

As used herein, an isoform refers to a protein that has an altered polypeptide structure compared to a full-length wildtype (predominant) form of the cognate protein due to a difference in the nucleic acid sequence and encoded polypeptide of the isoform compared to the corresponding protein. For purposes herein, isoforms include isoforms of a cell surface receptor (CSR) and isoforms of a ligand of a CSR. Generally an isoform provided herein lacks a domain or portion thereof (or includes insertions or both) sufficient to alter an activity, such as an enzymatic activity of a predominant form of the protein, or the structure of the protein. Reference herein to an isoform with altered activity refers to the alteration in an activity by virtue of the different structure or sequence of the isoform compared to a full-length or predominant form of the protein. With reference to an isoform, alteration of activity refers to a difference in activity between the particular isoforms and the predominant or wildtype form. Alteration of an activity includes an enhancement or a reduction of activity. In one embodiment, an alteration of an activity is a reduction in an activity; the reduction can be at least 0.1, 0.5, 1, 2, 3, 4, 5, or 10 fold compared to a wildtype and/or predominant form of the receptor. Typically, an activity is reduced 5, 10, 20, 50, 100 or 1000 fold or more. For example, a ligand can bind to a receptor and initiate or participate in signal transduction.

As used herein, a ligand isoform refers to a ligand that lacks a domain or portion of a domain or that has a disruption in a domain such as by the insertion of one or more amino acids compared to polypeptides of a wildtype or predominant form of the ligand. Typically such isoforms are encoded by alternatively spliced variants of the gene encoding the cognate receptor. Among the ligand isoforms provided herein are those that can bind to receptors but do not initiate signal transduction or initiate a reduced level of signal transduction. Such ligand isoforms act as ligand antagonists, and also process reduced activity as agonists compared to the wildtype ligand. A ligand isoform generally lacks a domain or portion thereof sufficient to alter an activity of a wildtype full-length and/or predominant form of the ligand, and/or modulates an activity of its receptor, or lacks a structural feature such as a domain. Such ligand isoforms, also include insertions and rearrangements. A ligand isoform includes those that exhibit activities that are altered from the corresponding wild-type ligand; for example, an isoform can include an alteration in a domain of the ligand so that it is unable to induce the dimerization of a receptor. In such an example, an isoform can compete for binding with a full-length wildtype ligand for its receptor, but reduce or inhibit signaling by the receptor. Generally, an activity is altered in an isoform at least 0.1, 0.5, 1, 2, 3, 4, 5, or 10 fold compared to a wildtype and/or predominant form of a ligand. Typically, an activity is altered by at least 2, 5, 10, 20, 50, 100, or 1000 fold or more. In one embodiment, alteration of an activity by a ligand isoform is a reduction in the activity compared to the predominant form of the ligand.

As used herein, a cell surface receptor (CSR) isoform, such as an isoform of a receptor tyrosine kinase, refers to a receptor that lacks a domain or portion thereof sufficient to alter or modulate an activity compared to a wildtype and/or predominant form of the receptor, or lacks a structural feature, such as a domain. A CSR isoform can include an isoform that has one or more biological activities that are altered from the receptor; for example, an isoform can include an alteration of the extracellular domain of p185-HER2, altering the isoform from a positively acting regulatory polypeptide of the receptor to a negatively acting regulatory polypeptide of the receptor, e.g. from a receptor domain into a ligand. Generally, an activity is altered in an isoform at least 0.1, 0.5, 1, 2, 3, 4, 5, or 10 fold compared to a wildtype and/or predominant form of the receptor. Typically, a activity is altered by at least 2, 5, 10, 20, 50, 100 or 1000 fold or more. In one embodiment, alteration of an activity is a reduction in the activity.

As used herein, an intron fusion protein refers to an isoform that lacks one or more domain(s) or portion of one or more domain(s). In addition, an intron fusion protein is encoded by nucleic acid molecules that contain one or more codons (with reference to the predominant or wildtype form of a protein), including stop codons, operatively linked to exon codons. The intron portion can be a stop codon, resulting in an intron fusion protein that ends at the exon intron junction. The activity of an intron fusion protein typically is different from the predominant form, generally by virtue of truncation(s), deletions and/or insertion of intron(s) amino acid residues. Such activities include changes in interaction with a receptor, or indirect changes that occur virtue of differences in interaction with a co-stimulating receptor or ligand, a receptor ligand or co-factor or other modulator of receptor activity. Intron fusion proteins isolated from cells or tissues or that have the sequence of such polypeptides isolated from cells or tissues, are “natural”. Those that do not occur naturally but that are synthesized or prepared by linking a molecule to an intron are referred to “synthetic” or “recombinant” or “combinatorial”. Included among intron fusion proteins are CSR isoforms or ligand isoforms that lack one or more domain(s) or a portion of one or more domain(s) resulting in an alteration of an activity of a cognate receptor or ligand by virtue of a change in the interaction between the intron fusion protein and its receptor or ligand or other interaction. Generally such isoforms are shortened compared to a wildtype or predominant form encoded by a CSR or ligand gene. They, however, can include insertions or other modifications in the exon portion and, thus, be of the same size or larger than the predominant form. Each, however, is encoded by a nucleic acid molecule that includes at least one codon (including stop codons) from an intron-encoded portion resulting either in truncation of the CSR or ligand isoform at the end of the exon or in an addition of one 2, 3, 4, 5, 8, 10, 15, 20, 25, 30, 35, 40, 45, 50, 55, 60, 65, 70, 75 and more amino acids encoded by an intron.

An intron fusion protein can be encoded by an alternatively spliced RNA and/or can be synthetically produced such as from RNA molecules identified in silico by identifying potential splice sites and then producing such molecules by recombinant methods. Typically, an intron fusion protein is shortened by the presence of one or more stop codons in an intron fusion protein-encoding RNA that are not present in the corresponding sequence of an RNA encoding a wildtype or predominant form of a corresponding polypeptide. If an intron includes an open reading frame in-frame with the exon portion, the intron encoded portion can be inserted in the polypeptide. Addition of amino acids and/or a stop codon results in an intron fusion protein that differs in size and sequence from a wildtype or predominant form of a polypeptide.

Intron fusion proteins for purposes herein include natural, combinatorial and synthetic intron fusion proteins. A natural intron fusion protein refers to a polypeptide that is encoded by an alternatively spliced RNA molecule that contains one or more amino acids encoded by an intron linked to one or more portions of the polypeptide encoded by one or more exons of a gene. Alternatively spliced mRNA is isolated or can be prepared synthetically by joining splice donor and acceptor sites in a gene. A natural intron fusion protein contains one or more amino acids or is truncated at the exon-intron junction because the intron contains a stop codon as the first codon. The natural intron fusion proteins generally occur in cells and/or tissues. Intron fusion proteins can be produced synthetically, for example based upon the sequence encoded by gene by identifying splice donor and acceptor sites and identifying possible encoded spliced variants. A combinatorial intron fusion protein refers to a polypeptide that is shortened compared to a wildtype or predominant form of a polypeptide. Typically, the shortening removes one or more domains or a portion thereof from a polypeptide such that an activity is altered. Combinatorial intron fusion proteins often mimic a natural intron fusion protein in that one or more domains or a portion thereof that is/are deleted in a natural intron fusion protein derived from the same gene or derived from a gene in a related gene family. Those that do not occur naturally but that are synthesized or prepared by linking a molecule to an intron such that the resulting construct modulates the activity of a CSR are “synthetic”.

As used herein, natural with reference to an intron fusion protein or a CSR or ligand isoform, refers to any protein, polypeptide or peptide or fragment thereof (by virtue of the presence of the appropriate splice acceptor/donor sites) that is encoded within the genome of an animal and/or is produced or generated in an animal or that could be produced from a gene. Natural intron fusion proteins include allelic variants and species variants. Intron fusion proteins can be modified post-translationally.

As used herein, an exon refers to a nucleic acid molecule containing a sequence of nucleotides that is transcribed into RNA and is represented in a mature form of RNA, such as mRNA (messenger RNA), after splicing and other RNA processing. An mRNA contains one or more exons operatively linked. Exons can encode polypeptides or a portion of a polypeptide. Exons also can contain non-translated sequences, for example, translational regulatory sequences. Exon sequences are often conserved and exhibit homology among gene family members.

As used herein, an intron refers to a sequence of nucleotides that is transcribed into RNA and is then typically removed from the RNA by splicing to create a mature form of an RNA, for example, an mRNA. Typically, nucleotide sequences of introns are not incorporated into mature RNAs, nor are intron sequences or a portion thereof typically translated and incorporated into a polypeptide. Splice signal sequences such as splice donors and acceptors are used by the splicing machinery of a cell to remove introns from RNA. It is noteworthy that an intron in one splice variant can be an exon (i.e., present in the spliced transcript) in another variant. Hence, spliced mRNA encoding an intron fusion protein can include an exon(s) and introns.

As used herein, splicing refers to a process of RNA maturation where introns in the mRNA are removed and exons are operatively linked to create a messenger RNA (mRNA).

As used herein, alternative splicing refers to the process of producing multiple mRNAs from a gene. Alternative splicing can include operatively linking less than all the exons of a gene, and/or operatively linking one or more alternate exons or introns that are not present in all transcripts derived from a gene.

As used herein, exon deletion refers to an event of alternative RNA splicing that produces a nucleic acid molecule that lacks at least one exon compared to an RNA molecule encoding a wildtype or predominant form of a polypeptide. An RNA molecule that has a deleted exon can be produced by such alternative splicing or by any other method, such as an in vitro method to delete the exon.

As used herein, exon insertion, refers to an event of alternative RNA splicing that produces a nucleic acid molecule that contains at least one exon not typically present in an RNA molecule encoding a wildtype or predominant form of a polypeptide. An RNA molecule that has an inserted exon can be produced by such alternative splicing or by any other method, such as an in vitro method to add or insert the exon.

As used herein, exon extension refers to an event of alternative RNA splicing that produces a nucleic acid molecule that contains at least one exon that is greater in length (number of nucleotides contained in the exon) than the corresponding exon in an RNA encoding a wildtype or predominant form of a polypeptide. An RNA molecule that has an extended exon can be produced by such alternative splicing or by any other method, such as an in vitro method to extend the exon. In some instances, as described herein, an mRNA produced by exon extension encodes an intron fusion protein.

As used herein, exon truncation refers to an event of alternative RNA splicing that produces a nucleic acid molecule that contains a truncation or shortening of one or more exons such that the one or more exons are shorter in length (number of nucleotides) compared to a corresponding exon in an RNA molecule encoding a wildtype or predominant form of a polypeptide. An RNA molecule that has a truncated exon can be produced by such alternative splicing or by any other method, such as an in vitro method to truncate the exon.

As used herein intron retention refers to an event of alternative RNA splicing that produces a nucleic acid molecule that contains an intron or a portion thereof operatively linked to one or more exons. An RNA molecule that retains an intron or portion thereof (generally an intron portion that encodes 1, 2, 3, 4, 5, 6, 7, 8, 9, 10, 11, 12, 13, 14, 15, or more codons including only a stop codon) can be produced by such alternative splicing or by any other method, such as in vitro methods to produce an RNA molecule with a retained exon. In some cases, as described herein, an mRNA molecule produced by intron retention encodes an intron fusion protein.

As used herein, a gene, also referred to as a gene sequence, refers to a sequence of nucleotides transcribed into RNA (introns and exons), including a nucleotide sequence that encodes at least one polypeptide. A gene includes sequences of nucleotides that regulate transcription and processing of RNA. A gene also includes regulatory sequences of nucleotides such as promoters and enhancers, and translation regulation sequences.

As used herein, a cognate gene with reference to an encoded polypeptide provided herein refers to the gene sequence that encodes a predominant polypeptide and is the same gene as the particular isoform. For purposes herein a cognate gene can include a natural gene or a gene that is synthesized such as by using recombinant DNA techniques. Generally, the cognate gene also is a predominant form in a particular cell or tissue.

As used herein, a splice site refers to one or more nucleotides within the gene that participate in the removal of an intron and/or the joining of an exon. Splice sites include splice acceptor sites and splice donor sites.

As used herein, an open reading frame refers to a sequence of nucleotides that encodes a functional polypeptide or a portion thereof, typically at least about fifty amino acids. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, truncation or shortening with reference to the shortening of a nucleic acid molecule or protein, refers to a sequence of nucleotides or amino acids that is less than full-length compared to a wildtype or predominant form of the protein or nucleic acid molecule.

As used herein, a reference gene refers to a gene that can be used to map introns and exons within a gene. A reference gene can be genomic DNA or a portion thereof, that can be compared with, for example, an expressed gene sequence, to map introns and exons in the gene. A reference gene also can be a gene encoding a wildtype or predominant form of a polypeptide.

As used herein, a premature stop codon is a stop codon occurring in the open reading frame of a sequence before the stop codon used to produce or create a full-length form of a protein, such as a wildtype or predominant form of a polypeptide. The occurrence of a premature stop codon can be the result of, for example, alternative splicing and mutation.

As used herein, an expressed gene sequence refers to any sequence of nucleotides transcribed or predicted to be transcribed from a gene. Expressed gene sequences include, but are not limited to, cDNAs, ESTs, and in silico predictions of expressed sequences, for example, based on splice site predictions and in silico generation of spliced sequences.

As used herein, an expressed sequence tag (EST) is a sequence of nucleotides generated from an expressed gene sequence. ESTs are generated by using a population of mRNA to produce cDNA. The cDNA molecules can be produced for example, by priming from the polyA tail present on mRNAs. cDNA molecules also can be produced by random priming using one or more oligonucleotides which prime cDNA synthesis internally in mRNAs. The generated cDNA molecules are sequenced and the sequences are typically stored in a database. An example of an EST database is dbEST found online at ncbi.nlm.nih.gov/dbEST. Each EST sequence is typically assigned a unique identifier and information such as the nucleotide sequence, length, tissue type where expressed, and other associated data is associated with the identifier.

As used herein, a cognate ligand with reference to the isoforms provided herein refers to the ligand that is encoded by the same gene as the particular isoform. Generally, the cognate ligand also is a predominant form in a particular cell or tissue.

As used herein, a wildtype form, for example, a wildtype form of a polypeptide, refers to a polypeptide that is encoded by a gene. Typically a wildtype form refers to a gene (or RNA or protein derived therefrom) without mutations or other modifications that alter function or structure; wildtype forms include allelic variation among and between species.

As used herein, a predominant form, for example, a predominant form of a polypeptide, refers to a polypeptide that is the major polypeptide produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form”.

As used herein, a domain refers to a portion (typically a sequence of three or more, generally 5 or 7 or more amino acids) of a polypeptide chain that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domains. For example, a domain can be identified, defined or distinguished by homology of the sequence therein to related family members, such as homology to motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, receptor tyrosine kinases typically include an extracellular domain, a membrane-spanning (transmembrane) domain and an intracellular tyrosine kinase domain.

As used herein, a polypeptide lacking all or a portion of a domain refers to a polypeptide that has a deletion of one or more amino acids or all of the amino acids of a domain compared to a cognate polypeptide. Amino acids deleted in a polypeptide lacking all or part of a domain can be contiguous, but need not be contiguous amino acids within the domain of the cognate polypeptide. Polypeptides that lack all or a part of a domain can exhibit a change, such as a loss or reduction of an activity of the polypeptide compared to the activity of a cognate polypeptide, or loss or addition of a structure in the polypeptide compared to a cognate polypeptide.

As used herein, a portion of a domain, such as a kringle domain, i.e. K4, or a sereine protease, i.e. SerP, includes at least one amino acid, typically, 2, 3, 4, 5, 6, 8, 10, 15 or more amino acids of the domain, but fewer than all of the amino acids that make up the domain. For example, if a cognate ligand has a Kringle domain, then a ligand isoform polypeptide lacking all or a part of the Kringle domain can have a deletion of 1, 2, 3, 4, 5, 6, 7, 8, 9, 10, 11, 12, 13, 14, 15, 16, 17, 18, 19, 20 or more amino acids of the amino acids corresponding to the same amino acid positions in the cognate ligand. Any isoform provided herein that contains such portion exhibits a desired activity such as, for example, modulation of the activity of a cell surface receptor.

As used herein, a polypeptide that contains a domain refers to a polypeptide that contains a complete domain with reference to the corresponding domain of a cognate ligand. A complete domain is determined with reference to the definition of that particular domain within a cognate polypeptide. For example, a ligand isoform comprising a domain refers to an isoform that contains a domain corresponding to the complete domain as found in the cognate ligand. If a cognate ligand, for example, contains a Kringle domain of 47 amino acids between amino acid positions 241-288, then a ligand isoform that comprises such Kringle domain, contains a 47 amino acid domain that has substantial identity with the 47 amino acid domain of the cognate ligand. Substantial identity refers to a domain that can contain allelic variation and conservative substitutions compared to the domain of the cognate ligand. Domains that are substantially identical do not have deletions, non-conservative substitutions or insertions of amino acids compared to the domain of the cognate ligand. Domains (i.e., a Kringle domain, a Serine Protease domain) often are identified by virtue of structural and/or sequence homology to domains in particular proteins.

Such domains are known to those of skill in the art who can identify such. For exemplification herein, definitions are provided, but it is understood that it is well within the skill in the art to recognize particular domains by name. If needed appropriate software can be employed to identify domains.

As used herein, an N-terminal domain belongs to the PAN module superfamily of domains which also includes the apple domains of the plasma prekallikrein/coagulation factor XI family, and domains of various nematode proteins. The PAN domain module contains a conserved core of three disulphide bridges. In some members of the family there is an additional fourth disulphide bridge that links the N and C termini of the domain. The domain is found in diverse proteins. In some the domain mediates protein-protein interactions, in others it mediates protein-carbohydrate interactions. HGF contains an N-terminal domain which binds to the MET receptor and to the heparin molecule. The structure of the N-terminal domain of HGF contains a characteristic hairpin-loop structure stabilized by two disulfide bridges.

As used herein, a kringle domain contains about 80 amino acids and has a characteristic folding pattern defined by three internal disulfide bonds and additional conserved residues. Generally, kringle domains are involved in protein-protein interactions. An exemplary HGF provided herein as set forth in SEQ ID NO:3 contains four kringle domains.

As used herein, a serine protease domain refers generally to a large group of peptidases which share a common closed beta barrel structure. Typically, a protease domain is the catalytically active portion of a protease. A protease domain of a protein contains all of the requisite properties of that protein required for its proteolytic activity, such as for example, its catalytic center. The catalytic center of a serine protease is a catalytic triad of three amino acids, an aspartic acid, a histidine, and a serine. In the exemplary HGF provided herein, residues in the catalytic triad are mutated such that the protein does not have proteolytic activity.

As used herein, an allelic variant or allelic variation refers to a polypeptide encoded by a gene that differs from a reference form of a gene (i.e. is encoded by an allele) among a population. Typically the reference form of the gene encodes a wildtype form and/or predominant form of a polypeptide from a population or single reference member of a species. Typically, allelic variants have at least 80%, 90%, 95% or greater amino acid identity with a wildtype and/or predominant form from the same species.

As used herein, species variants refers to variants of the same polypeptide between and among species. Generally, interspecies allelic variants have at least about 60%, 70%. 80%, 85%, 90% or 95% identity or greater with a wildtype and/or predominant form of another species, including 96%, 97%, 98%, 99% or greater identity with a wildtype and/or predominant form of a polypeptide.

As used herein, modification refers to modification of a sequence of amino acids of a polypeptide or a sequence of nucleotides in a nucleic acid molecule and includes deletions, insertions, and replacements of amino acids and nucleotides, respectively.

As used herein, designated refers to the selection of a molecule or portion thereof as a point of reference or comparison. For example, a domain can be selected as a designated domain for the purpose of constructing polypeptides that are modified within the selected domain. In another example, an intron can be selected as a designated intron for the purpose of identifying RNA transcripts that include or exclude the selected intron.

As used herein, an agonist refers to a molecule that elicits a maximal response by a receptor.

As used herein, a partial agonist refers to a molecule that elicits a response by a receptor, however, the maximum response obtained is less that that of an agonist (e.g., the physiological ligand).

As used herein, an antagonist or competitive antagonist refers to a molecule that competes with a wildtype or predominant ligand for receptor binding, without itself leading to activation of the receptor.

As used herein, a ligand antagonist refers to the activity of an isoform that antagonizes an activity that results from ligand interaction with a CSR.

As used herein, inhibit and inhibition refer to a reduction in an activity, such as a biological activity, relative to the uninhibited activity.

As used herein, an activity refers to a function or functioning or changes in or interactions of a biomolecule, such as polypeptide. Exemplary, but not limiting of such activities are: complexation, dimerization, multimerization, receptor-associated kinase activity or other enzymatic or catalytic activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation (mitogenesis), migration (motogenesis), differentiation (morphogenesis), angiogenesis, growth, degradation, membrane localization, membrane binding, and oncogenesis. An activity can be assessed by assays described herein and by any suitable assays known to those of skill in the art, including, but not limited to, in vitro assays, including cell-based assays, in vivo assays, including assays in animal models for particular diseases. Biological activities refer to activities exhibited in vivo. For purposes herein, biological activity refers to any of the activities exhibited by a polypeptide provided herein.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein-protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules of the same type, such as two molecules of a receptor. Dimerization includes homodimerization where two identical molecules interact. Dimerization also includes heterodimerization of two different molecules, such as two subunits of a receptor and dimerization of two different receptor molecules. Typically, dimerization involves two molecules that interact with each other through interaction of a dimerization domain contained in each molecule.

As used herein, mitogenesis refers to a process by which an agent induces mitosis and cell proliferation.

As used herein, motogenesis refers to the process of regulating cell movement or migration and generally implies regulated movement of a population of cells from one place to another.

As used herein, morphogenesis refers to the differentiation and growth of cells, tissues or organs. Differentiation can occur during the formation of the structure of an organism or part, such as during organogenesis. Differentiation can also occur at the cellular level, such as when a cell undergoes a change toward a more specialized form or function.

As used herein, angiogenesis refers to the formation of new blood vessels.

As used herein, an “anti-angiogenic” or “angio-inhibitory” molecule refers to a molecule that inhibits angiogenesis.

As used herein, modulate and modulation refer to a change of an activity of a molecule, such as a protein. Exemplary activities include, but are not limited to, activities such as signal transduction and protein phosphorylation. Modulation can include an increase in the activity (i.e., up-regulation of agonist activity), a decrease in activity (i.e., down-regulation or inhibition) or any other alteration in an activity (such as in periodicity, frequency, duration and kinetics.) Modulation can be context dependent and typically modulation is compared to a designated state, for example, the wildtype protein, the protein in a constitutive state, or the protein as expressed in a designated cell type or condition.

As used herein, reference to modulating the activity of a cell surface receptor means that a CSR or ligand isoform interacts in some manner with the receptor, whereby an activity, such as, but not limited to ligand binding, dimerization and/or other signal-transduction-related activity, is altered.

As used herein, reference to a ligand isoform, including an HGF isoform, with altered activity refers to an alteration in an activity by virtue of the different structure or sequence of the CSR or ligand isoform compared to a cognate receptor or ligand.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related. A kit is a packaged combination that optionally includes instructions for use of the combination or elements thereof and/or optionally includes other reagents and vessels and tools and devices employed in the methods for which the kit is intended.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein, a disease involving HGF or an HGF-mediated disease refers to any disease in which HGF plays a role, whereby, modulation of its activity would effect treatment of the disease or a symptom of the disease. Included among HGF-mediated diseases are MET-mediated diseases involving HGF-MET signaling, as well as other angiogenic diseases involving signaling by other CSRs, including FGFR or VEGFR. Exemplary of such diseases include cancers and other diseases involving undesirable cell proliferation, angiogenic and inflammatory reactions or responses.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to animals, including mammals, such as human beings.

As used herein, a patient refers to a human subject.

As used herein, angiogenic diseases (or angiogenesis-related diseases) are diseases in which the balance of angiogenesis is altered or the timing thereof is altered. Angiogenic diseases include those in which an alteration of angiogenesis, such as undesirable vascularization, occurs. Such diseases include, but are not limited to, cell proliferative disorders, including cancers, diabetic retinopathies and other diabetic complications, inflammatory diseases, endometriosis and other diseases in which excessive vascularization is part of the disease process, including those noted above.

As used herein, in silico refers to research and experiments performed using a computer. In silico methods include, but are not limited to, molecular modeling studies, biomolecular docking experiments, and virtual representations of molecular structures and/or processes, such as molecular interactions.

As used herein, biological sample refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or to a sample that is processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi, algae, protozoa and components thereof.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, a biomolecule is any compound found in nature, or derivatives thereof. Exemplary biomolecules include but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide”, although those in the art recognize that oligonucleotides, for example, PCR primers, generally are less than about fifty to one hundred nucleotides in length.

Polynucleotides can include nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, synthetic, in the context of a synthetic sequence and synthetic gene refers to a nucleic acid molecule that is produced by recombinant methods and/or by chemical synthesis methods.

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, production by recombinant means by using recombinant DNA methods refers to the use of the well-known methods of molecular biology for expressing proteins encoded by cloned DNA.

As used herein, “isolated”, with reference to a molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated”. Likewise, a molecule that has been purified, partially or substantially, from a native source or recombinant host cell, or produced by synthetic methods, is considered “isolated”. Depending on the intended application, an isolated molecule can be present in any form, such as in an animal, cell or extract thereof; dehydrated, in vapor, solution or suspension; or immobilized on a solid support.

As used herein, the term “vector” refers to a nucleic acid molecule capable of transporting another nucleic acid to which it has been linked. One type of vector is an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors”. In general, expression vectors are often in the form of “plasmids,” which are generally circular double stranded DNA loops that, in their vector form, are not bound to the chromosome. “Plasmid” and “vector” are used interchangeably as the plasmid is the most commonly used form of vector. Other forms of expression vectors include those that serve equivalent functions and that become known in the art subsequently hereto.

As used herein, “transgenic animal” refers to any animal, generally a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule can be stably integrated within a chromosome, i.e., replicate as part of the chromosome, or it can be extrachromosomally replicating DNA. In the typical transgenic animals, the transgene causes cells to express a recombinant form of a protein.

As used herein, a reporter gene construct is a nucleic acid molecule that includes a nucleic acid encoding a reporter operatively linked to transcriptional control sequences. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene”, can be expressed as a fusion protein with a protein of interest or under to the control of a promoter of interest.

As used herein, the phrase “operatively linked” with reference to sequences of nucleic acids means the nucleic acid molecules or segments thereof are covalently joined into one piece of nucleic acid such as DNA or RNA, whether in single or double stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control of regulatory sequences on one segment control expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way as to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, the term “operatively linked” with reference to amino acids in polypeptides refers to covalent linkage (direct or indirect) of the amino acids. For example, at least one domain of a ligand, such as HGF, operatively linked to at least one amino acid encoded by an intron of a gene encoding a ligand, means that the amino acids of a domain from a ligand are covalently joined to amino acids encoded by an intron from a ligand gene. Such linkage, typically direct via peptide bonds, also can be effected indirectly, such as via a linker or via non-peptidic linkage. Hence, a polypeptide that contains at least one domain of a ligand operatively linked to at least one amino acid encoded by an intron of a gene encoding a cell surface receptor can be an intron fusion protein. Nucleic acids encoding such polypeptides can be produced when an intron sequence is spliced or otherwise covalently joined in-frame to an exon sequence that encodes a domain of a cell surface receptor. Translation of the nucleic acid molecule produces a polypeptide where an intron-encoded portion of amino acid(s), minimally containing a stop codon encoded by the intron sequence, are covalently joined to a domain of the ligand. They also can be produced synthetically by linking a portion containing an exon to a portion containing an intron, including chimeric intron fusion proteins in which the exon is encoded by a gene for a different isoform, including a different ligand isoform or cell surface receptor isoform, from the intron portion or vice versa.

As used herein, the phrase “generated from a nucleic acid” in reference to the generation of a polypeptide, such as an isoform and intron fusion protein, includes the literal generation of a polypeptide molecule and the generation of an amino acid sequence of a polypeptide from translation of the nucleic acid sequence into a sequence of amino acids.

As used herein, a conjugate refers to the joining together of a nucleic acid or polypeptide. Conjugation can be effected directly or indirectly. In some examples, linkers can be used such as peptide linkers, restriction enzyme linkers, or other linkers. Conjugation can also be effected chemically, such as by using heterobifunctional cross-linking reagents.

As used herein, cross-linking refers to the process of chemically joining two or more molecules by a covalent bond. Cross-linking reagents contain reactive ends to specific functional groups (primary amines, sulfhydryls, etc.) on proteins or other molecules. Cross-linkers include homo- and heterobifunctional cross-linkers. Homobifunctional cross-linkers have two identical reactive groups and often are used in one-step reaction procedures to cross-link proteins to each other or to stabilize quaternary structure. Heterobifunctional cross-linkers possess two different reactive groups that allow for sequential (two-stage) conjugations, helping to minimize undesirable polymerization or self-conjugation.

As used herein, a fusion protein refers to a protein created through recombinant DNA techniques and is achieved by operatively linking all or part of the nucleic acid sequence of one gene with all or part of the nucleic acid sequence of another gene. In some cases, a fusion can encode a chimeric protein containing two or more proteins or peptides.

As used herein, production with reference to a polypeptide refers to expression and recovery of an expressed protein (or recoverable or isolatable expressed protein). Factors that can influence the production of a protein include the expression system and host cell chosen, the cell culture conditions, the secretion of the protein by the host cell, and ability to detect a protein for purification purposes. Production of a protein can be monitored by assessing the secretion of a protein, such as for example, into cell culture medium.

As used herein, “improved production” refers to an increase in the production of a polypeptide compared to production of a control polypeptide. For example, production of an isoform fusion protein is compared to a corresponding isoform that is not a fusion protein or that contains a different fusion. For example, the production of an isoform containing a tPA pre/prosequence can be compared to an isoform containing its endogenous signal sequence. Generally, production of a protein can be improved more than, about or at least 1, 1.5, 2, 3, 4, 5, 6, 7, 8, 9 10 fold and more. Typically, production of a protein can be improved by 5, 10, 20, 30, 40, 50 fold or more compared to a corresponding isoform that is not an isoform fusion or does not contain the same fusion.

As used herein, secretion refers to the process by which a protein is transported into the external cellular environment or, in the case of gram-negative bacteria, into the periplasmic space. Generally, secretion occurs through a secretory pathway in a cell, for example, in eukaryotic cells this involves the endoplasmic reticulum and golgi apparatus.

As used herein, a “precursor sequence” or “precursor peptide” or “precursor polypeptide” refers to a sequence of amino acids, that is processed, and that occurs at a terminus, typically at the amino terminus, of a polypeptide prior to processing or cleavage. The precursor sequence includes sequences of amino acids that effect secretion and/or trafficking of the linked polypeptide. The precursor sequence can include one or more functional portions. For example, it can include a presequence (a signal polypeptide) and/or a prosequence. Processing of a polypeptide into a mature polypeptide results in the cleavage of a precursor sequence from a polypeptide. The precursor sequence, when it includes a presequence and a prosequence also can be referred to as a pre/prosequence.

As used herein, a “presequence”, “signal sequence”, “signal peptide”, “leader sequence” or “leader peptide” refers to a sequence of amino acids at the amino terminus of nascent polypeptides, which target proteins to the secretory pathway and are cleaved from the nascent chain once translocated in the endoplasmic reticulum membrane.

As used herein, a prosequence refers to a sequence encoding a propeptide which, when it is linked to a polypeptide, can exhibit diverse regulatory functions including, but not limited to, contributing to the correct folding and formation of disulfide bonds of a mature polypeptide, contributing to the activation of a polypeptide upon cleavage of the pro-peptide, and/or contributing as recognition sites. Generally, a pro-sequence is cleaved off within the cell before secretion, although it also can be cleaved extracellularly by exoproteases. In some examples, a pro-sequence is autocatalytically cleaved while in other examples another polypeptide protease cleaves a pro-sequence.

As used herein, homologous refers to a molecule, such as a nucleic acid molecule or polypeptide, from different species that correspond to each other and that are identical or very similar to each other (i.e., are homologs).

As used herein, heterologous refers to a molecule, such as a nucleic acid or polypeptide, that is unique in activity or sequence. A heterologous molecule can be derived from a separate genetic source or species. For purposes herein, a heterologous molecule is a protein or polypeptide, regardless of origin, other than a CSR or ligand isoform, or allelic variants thereof. Thus, molecules heterologous to a CSR or ligand isoform include any molecule containing a sequence that is not derived from, endogenous to, or homologous to the sequence of a CSR or ligand isoform. Examples of heterologous molecules of interest herein include secretion signals from a different polypeptide of the same or different species, a tag such as a fusion tag or label, or all or part of any other molecule that is not homologous to and whose sequence is not the same as that of a CSR isoform or ligand. A heterologous molecule can be fused to a nucleic acid or polypeptide sequence of interest for the generation of a fusion or chimeric molecule.

As used herein, a heterologous secretion signal refers to the a signal sequence from a polypeptide, from the same or different species, that is different in sequence from the signal sequence of a CSR or ligand isoform. A heterologous secretion signal can be used in a host cell from which it is derived or it can be used host cells that differ from the cells from which the signal sequence is derived.

As used herein, an endogenous precursor sequence or endogenous signal sequence refers to the naturally occurring signal sequence associated with all or part of a polypeptide. The approximate location of signal sequence of CSR and ligand isoforms, based on their corresponding cognate receptor or ligand signal sequence, are known to one of skill in the art. The C-terminal boundary of a signal peptide may vary, however, typically by no more than about 5 amino acids on either side of the signal peptide C-terminal boundary. Algorithms are available and known to one of skill in the art to identify signal sequences and predict their cleavage site (see e.g., Chou et al., (2001), Proteins 42:136; McGeoch et al., (1985) Virus Res. 3:271; von Heijne et al., (1986) Nucleic Acids Res. 14:4683).

As used herein, tissue plasminogen activator (tPA) refers to an extrinsic (tissue-type) plasminogen activator having fibrinolytic activity and typically having a structure with five domains (finger, growth factor, kringle-1, kringle-2, and protease domains). Mammalian t-PA includes t-PA from any animals, including humans. Other species include, but are not limited to, rabbit, rat, porcine, non human primate, equine, murine, dog, cat, bovine and ovine tPA. Nucleic acid encoding tPA including the precursor polypeptide(s) from human and non-human species is known in the art.

As used herein, a tPA precursor sequence refers to a sequence of amino acid residues that includes the presequence and prosequence from tPA (i.e., is a pre/prosequence, see e.g., U.S. Pat. No. 6,693,181 and U.S. Pat. No. 4,766,075). This polypeptide is naturally associated with tPA and acts to direct the secretion of a tPA from a cell. An exemplary precursor sequence for tPA is set forth in SEQ ID NO:253 and encoded by a nucleic acid sequence set forth in SEQ ID NO:252. The precursor sequence includes the signal sequence (amino acids 1-23) and a prosequence (amino acids 24-35). The prosequence includes two protease cleavage sites: one after residue 32 and another after residue 35. Exemplary species variants of precursor sequences are set forth in any one of SEQ ID NOS: 258-265; exemplary nucleotide and amino acid allelic variants are set forth in SEQ ID NOS:256 or 257.

As used herein, all or a portion of a tPA precursor sequence refers to any contiguous portion of amino acids of a tPA precursor sequence sufficient to direct processing and/or secretion of tPA from a cell. All or a portion of a precursor sequence can include all or a portion of a wildtype or predominant tPA precursor sequence such as set forth in SEQ ID NO:253 and encoded by SEQ ID NO:252, allelic variants thereof set forth in SEQ ID NO: 257, or species variants set forth in SEQ ID NOS:256-265. For example, for the exemplary tPA precursor sequence set forth in SEQ ID NO:253, a portion of a tPA precursor sequence can include amino acids 1-23, or amino acids 24-35, 24-32, or amino acids 33-35, or any other contiguous sequence of amino acids 1-35 set forth in SEQ ID NO:253.

As used herein, an active portion of a polypeptide, such as with reference to an active portion of an isoform, refers to a portion of polypeptide that has an activity.

As used herein, purification of a protein refers to the process of isolating a protein, such as from a homogenate, which can contain cell and tissue components, including DNA, cell membrane and other proteins. Proteins can be purified in any of a variety of ways known to those of skill in the art, such as for example, according to their isoelectric points by running them through a pH graded gel or an ion exchange column, according to their size or molecular weight via size exclusion chromatography or by SDS-PAGE (sodium dodecyl sulfate-polyacrylamide gel electrophoresis) analysis, or according to their hydrophobicity. Other purification techniques include, but are not limited to, precipitation or affinity chromatography, including immuno-affinity chromatography, and other techniques and methods that include a combination of any of these methods. Furthermore, purification can be facilitated by including a tag on the molecule, such as a his tag for affinity purification or a detectable marker for identification.

As used herein, detection includes methods that permit visualization (by eye or equipment) of a protein. A protein can be visualized using an antibody specific to the protein. Detection of a protein can also be facilitated by fusion of a protein with a tag including an epitope tag or label.

As used herein, a “tag” refers to a sequence of amino acids, typically added to the N- or C-terminus of a polypeptide. The inclusion of tags fused to a polypeptide can facilitate polypeptide purification and/or detection.

As used herein, an epitope tag includes a sequence of amino acids that has enough residues to provide an epitope against which an antibody can be made, yet short enough so that it does not interfere with an activity of the polypeptide to which it is fused. Suitable tag polypeptides generally have at least 6 amino acid residues and usually between about 8 and 50 amino acid residues.

As used herein, a label refers to a detectable compound or composition which is conjugated directly or indirectly to an isoform so as to generate a labeled isoform. The label can be detectable by itself (e.g., radioisotope labels or fluorescent labels) or, in the case of an enzymatic label, can catalyze chemical alteration of a substrate compound composition which is then detectable. Non-limiting examples of labels included fluorogenic moieties, green fluorescent protein, or luciferase.

As used herein, a fusion tagged polypeptide refers to a chimeric polypeptide containing an isoform polypeptide fused to a tag polypeptide.

As used herein, expression refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNA). For purposes herein, a protein that is expressed can be retained inside the cells, such as in the cytoplasm, or can be secreted from the cell. Total expression of a protein

As used herein, a fusion construct refers to a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, but typically fewer than 10, 9, 8, 7, 6 amino acids. The protein product encoded by a fusion construct is referred to as a fusion polypeptide.

As used herein, an isoform fusion protein or an isoform fusion polypeptide refers to a polypeptide encoded by a nucleic acid molecule that contains a coding sequence from an isoform, with or without an intron sequence, and a coding sequence that encodes another polypeptide, such as a precursor sequence or an epitope tag. The nucleic acids are operatively linked such that when the isoform fusion construct is transcribed and translated, an isoform fusion polypeptide is produced in which the isoform polypeptide is joined directly or via a linker to another peptide. An isoform polypeptide, typically is linked at the N—, or C-terminus, or both, to one or more other polypeptides.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the “amino acids,” which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§. 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH2 or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions may be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, a peptidomimetic is a compound that mimics the conformation and certain stereochemical features of a biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics can be prepared from biologically active compounds by replacing certain groups or bonds that contribute to the undesirable properties with bioisosteres. Bioisosteres are known to those of skill in the art. For example the methylene bioisostere CH2S has been used as an amide replacement in enkephalin analogs (see, e.g., Spatola (1983) pp. 267-357 in Chemistry and Biochemistry of Amino Acids, Peptides, and Proteins, Weinstein, Ed. volume 7, Marcel Dekker, New York). Morphine, which can be administered orally, is a compound that is a peptidomimetic of the peptide endorphin. For purposes herein, polypeptides in which one or more peptidic bonds that form the backbone of a polypeptide are replaced with bioisosteres are peptidomimetics.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carrillo, H. & Lipman, D., SIAM J Applied Math 48:1073 (1988)).

As used herein, sequence identity compared along the full length of a polypeptide compared to another polypeptide refers to the percentage of identity of an amino acid in a polypeptide along its full-length. For example, if a polypeptide A has 100 amino acids and polypeptide B has 95 amino acids identical to amino acids 1-95 of polypeptide A, then polypeptide B has 95% identity when sequence identity is compared along the full length of a polypeptide A compared to full length of polypeptide B. As discussed below, and known to those of skill in the art, various programs and methods for assessing identity are known to those of skill in the art. High levels of identity, such as 90% or 95% identity, readily can be determined without software.

As used herein, by homologous (with respect to nucleic acid and/or amino acid sequences) means about greater than or equal to 25% sequence homology, typically greater than or equal to 25%, 40%, 60%, 70%, 80%, 85%, 90% or 95% sequence homology; the precise percentage can be specified if necessary. For purposes herein, the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two nucleic acid molecules have nucleotide sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Altschul, S. F., et al., J Molec Biol 215:403 (1990); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes, a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used here, the singular forms “a,” “an” and “the” include plural referents unless the context clearly dictates otherwise. Thus, for example, reference to compound, comprising “an extracellular domain”” includes compounds with one or a plurality of extracellular domains.

As used herein, ranges and amounts can be expressed as “about” a particular value or range. About also includes the exact amount. Hence “about 5 bases” means “about 5 bases” and also “5 bases.’

As used herein,, “optional” or “optionally” means that the subsequently described event or circumstance does or does not not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. Hepatocyte Growth Factor (HGF) and Met Receptor

Provided herein are isoforms of Hepatocyte Growth Factor (HGF). The HGF isoforms differ from the cognate ligand in that there are insertions and/or deletions so the resulting HGF isoforms exhibit a difference in one or more activities or functions or in structure compared to HGF. Activities or functions include, but are not limited to, receptor dimerization, cell signaling, cell migration, cell growth and proliferation, and angiogenesis. HGF isoforms have a plurality of activities, including activities as modulators of the HGF receptor, MET, and angioinhibitory activities. Among the HGF isoforms provided are those that modulate the activities of other growth factor receptors, such as VEGFR or FGFR by modulation of a VEGFR ligand or FGFR ligand, and also include HGF isoforms with general angioinhibitory activity. Among the HGF isoforms provided herein are those that exhibit MET receptor antagonist activity and also display anti-angiogenic activities.

1. HGF

Hepatocyte growth factor (HGF, also called Scatter Factor, SF and Hepatopoeitin A) is a pleiotropic factor that targets a variety of epithelial and endothelial cells. HGF plays a role in organ regeneration, organogenesis, embryogenesis, and carcinogenesis. It has mitogenic, motogenic (enhancement of cell motility), and morphogenic activities. Particular physiologic functions of HGF include supporting organogenesis of various organs by mediating epithelial-mesenchymal interactions, and stimulating neovascularization in tumors by mediating tumor-stromal interaction. HGF contains a protease domain homologous to the catalytic domain of other serine proteases, such as for example, plasminogen, tPA, uPA, and factor XII. HGF, however, does not display protease activity due to alterations in two of the three amino acids that make up the catalytic triad (i.e. H534Q and S673Y).

An exemplary human HGF is encoded by a single open reading frame precursor of 728 amino acids containing a signal sequence at N-terminal amino acids 1-31. The mature HGF protein is proteolytically processed to a disulfide-linked heterodimer molecule composed of a 69 kDa alpha-chain (also called the heavy chain of the dimer) extending from amino acids 32 to 494 of the exemplary HGF set forth as SEQ ID NO:3 and a 34 kDa beta-chain (also called the light chain of the dimer) extending from amino acids 495 to 728 of the exemplary HGF set forth as SEQ ID NO:3. The alpha-chain of the HGF molecule contains four kringle structures that function as protein binding modules, and the beta-chain contains a serine protease (SerP)-like domain (see e.g., FIG. 2). For example, in the exemplary full-length HGF polypeptide provided herein as SEQ ID NO:3, and encoded by SEQ ID NO:2, the signal peptide is located at amino acids 1-31, an N-terminal domain is located at amino acids 34 to 124, a Kringle 1 domain is located at amino acids 128 to 206, a Kringle 2 domain is located at amino acids 211 to 288, a Kringle 3 domain is located at amino acids 305 to 383, a Kringle 4 domain is located at amino acids 391 to 469, an interchain between the alpha and beta chain is located between amino acids 487-604, and a serine protease (SerP, peptidase S1) domain is located at amino acids 495 to 728.

The HGF gene (SEQ ID NO:1) is composed of 18 exons interrupted by 17 introns (see e.g., FIG. 1). Exon 1 of HGF contains the 5′-untranslated region and signal peptide associated with secretion, exon 2 and exon 3 encode the N domain which is a hairpin loop region stabilized by two disulfide bonds, exon 4-11 encode the four kringles, each kringle being encoded by two exons, exon 12 contains the spacer between the alpha- and beta-chains, and the remaining six exons encode a SerP-like domain (see, e.g., Seki et al., (1991) Gene 102:213). In the exemplary genomic sequence of HGF provided herein as SEQ ID NO:1, exon 1 includes nucleotides 1-253, with the start codon beginning at nucleotide position 166; intron 1 includes nucleotides 254-7264; exon 2 includes nucleotides 7265-7431; intron 2 includes nucleotides 7432-11333; exon 3 includes nucleotides 11334-11445; intron 3 includes nucleotides 11446-12833; exon 4 includes nucleotides 12834-12948; intron 4 includes nucleotides 12949-17874; exon 5 includes nucleotides 17875-18117; intron 5 includes nucleotides 18118-25016; exon 6 includes nucleotides 25017-25137; intron 6 includes nucleotides 25138-26665; exon 7 includes nucleotides 26666-26784; intron 7 includes nucleotides 26785-40357; exon 8 includes nucleotides 40358-40532; intron 8 includes nucleotides 40533-44119; exon 9 includes nucleotides 44120-44247; intron 9 includes nucleotides 44248-49289; exon 10 includes nucleotides 49290-49392; intron 10 includes nucleotides 49393-52771; exon 11 includes nucleotides 52772-52905; intron 11 includes nucleotides 52906-58617; exon 12 includes nucleotides 58618-58656; intron 12 includes nucleotides 58657-59893; exon 13 includes nucleotides 59894-59991; intron 13 includes nucleotides 59992-62772; exon 14 includes nucleotides 62773-62847; intron 14 includes nucleotides 62848-63709; exon 15 includes nucleotides 63710-63850; intron 15 includes nucleotides 63851-64383; exon 16 includes nucleotides 64384-64490; intron 16 includes nucleotides 64491-64601; exon 17 includes nucleotides 64602-64747; intron 17 includes nucleotides 64748-67379; and exon 18 includes nucleotides 67380-68009.

HGF participates in a variety of its activities through modulation of the receptor designated MET. These activities include those associated with motility, mitogenesis, and morphogenesis of cells, including cancer cells, as well as the promotion of angiogenesis. For example, HGF acts as a mitogenic factor for hepatocytes (Nakamura et al. (1991), Prog. in Growth Factor Res. 3:67), epithelial cells (Dignass et al., (1994) Biochem. Biophys. Res. Comm. 202:701), endothelial cells (Bussolino et al., (1992) J. Cell Biol. 119:629), dermal fibroblasts (Kataoka et al., (1993) Cell Biol. Internat. 17:65), melanocytes (Matsumoto et al., (1991) Biochem. Biophys. Res. Comm. 176:45), and hematopoietic precursor cells (Kmiecik et al., (1992) Blood 80:2454). In addition, HGF acts as a motogenic factor for endothelial cells and many epithelial cells, including hepatocytes and for several tumor cells enhancing cellular invasiveness (Stoker et al., (1987) Nature 327:239; Weidner et al., (1991) Proc. Natl. Acad. Sci. 88:7001). HGF also acts as a morphogenic factor to induce tubule formation by kidney epithelial cells (Montesano et al., (1991) Cell 67:901), ductule formation by mammary epithelial cells (Tsafarty et al., (1992) Science 257:1258), and cord formation by hepatocytes (Michalopoulos et al., (1993) Am. J. Physiol. 156:443). Other properties of HGF include activity as a cytotoxic or cytostatic factor, such as for example in tumor cells (Shiota et al., (1992) Proc. Natl. Acad. Sci. 89:373), and as an angiogenic factor (Morishita et al., (2004) Curr Gene Ther. 4:199). Additionally, HGF displays immunoregulatory activities such as suppressing dendritic cell function (Okunishi et al., (2005) J Immunol. 175:4745).

Some HGF-mediated activities are induced upon binding and tyrosine-autophosphorylation of its receptor, MET, followed by the recruitment of a group of signaling molecules and/or adaptor proteins to the cytoplasmic domain of MET leading to the activation of multiple signaling cascades that form a complete network of intra and extracellular responses. Upon HGF binding, MET engages a number of SH2-containing signal transducers, including phosphotidylinositol 3-kinase, phospholipase C-γ, Stat3, Grb2, and the Grb2-associated docking protein Gab1, and indirectly activates the Ras-mitogen-activated protein kinase (MAPK) pathway. Different combinations of signaling pathways and signaling molecules and/or differences in magnitude of responses contribute to the diverse activities of HGF/MET. Further, the activity of HGF is influenced by cell type as well as different cellular environments.

The mechanism of MET activation by HGF requires cleavage of the single-chain HGF into a two-chain form. The single-chain form of HGF retains receptor binding, but lacks the biological activity of the two-chain form of HGF, and thus functions as an antagonist of HGF activity. It is likely that cleavage into a two-chain form results in a conformational change and a possible rearrangement of domains (Chirgadze et al., (1998) FEBS Letters 430: 126). Typically, activation of receptor tyrosine kinases, such as MET, requires a transition from a monomeric to dimeric state upon binding of their cognate ligand. Consequently, the ligand must either possess two binding sites or be a dimer itself. It is postulated that the conformational change of HGF into a two-chain form permits HGF to dimerize before receptor activation. Interactions between the SerP domains can stabilize the interaction of the dimer, since the SerP domain is critical for biological activity, but not receptor binding, of HGF. Heparin and heparin sulfates can further stabilize the full-length dimer, and are critical for crosslinking natural HGF isoform monomers, NK1 and NK2, for agonist activity (Chirgadze et al., (1998) FEBS Letters 430: 126).

a. HGF Domain Structure

Structure-function studies have elucidated functions of the HGF domains. Deletion of either the hairpin loop of the N-terminal domain, kringles 1 or 2, or the SerP domain abolishes the biological activity of HGF. In contrast, molecules with deletions of kringle 3 or kringle 4 display reduced but measurable activity (Chirgadze et al., (1998) FEBS Letters 430:126). The α-chain of HGF is responsible for binding to the MET receptor, and this interaction is primarily mediated by the N-terminal domain and the first kringle (K1) domain.

i. N Terminal Domain

The N-terminal domain, containing amino acids 34 to 124 of an exemplary HGF set forth in SEQ ID NO:3, is implicated in binding to heparin sulfate glycosaminoglycans (HSGAGs) on the surface of cells which is required for high affinity interactions with its receptor MET. Typically, binding of a ligand to HSGAGs or soluble heparin promotes the stabilization and/or localization of a ligand with a less abundant higher affinity tyrosine kinase receptor involved in signal transduction. Heparin binding promotes ligand oligomerization which can enhance signaling by stimulating dimerization of the tyrosine kinase receptor. Various growth factors, such as HGF, FGF1 and FGF2 rely on heparin-containing coreceptors to provide secondary binding sites that complement the interaction of the specific receptor and strengthen adhesive forces. For example, treatment of cells with heparitinase, which cleaves HSGAGs from the cell surface, diminishes HGF-MET crosslinking and administration of soluble heparin to cells alters HGF-mediated functions (Sakata et al., (1997) J Biol Chem., 272:9457). The heparin binding site of HGF is made up of basic and/or polar residues in the N-terminal domain of HGF (Zhou et al., (1998) Structure, 6:109) and studies have shown that the addition of heparin to a recombinant N-terminal domain, but not to a recombinant K1 domain, is sufficient to induce oligomerization of the domain (Sakata et al., (1997) J Biol Chem., 272:9457). Consequently, the N-terminal domain of HGF retains heparin or endogenous HSGAG binding ability required for ligand oligomerization, receptor binding, and receptor activation and signaling. The requirement for the N-terminal domain of HGF for binding of its receptor MET has implicated the N-terminal domain as a critical determinant of the antagonistic activity of the engineered HGF isoform NK4 (see below, (Kuba et al., (2000) Biochem. Biophys. Res. Commun. 279:846).

In addition to promoting receptor dimerization through interactions with heparin, interactions of the N-terminal domain with heparin sulfate also play a role in receptor-independent angiogenesis inhibition. A recombinant peptide of the HGF N-terminal domain inhibits angiogenesis not by disrupting the HGF/MET interaction, but rather by interfering with binding of HGF to endothelial GAGs, including HSGAG. Moreover, the anti-angiogenic role of the HGF N-terminal domain is not restricted to HGF since the N-terminal domain can antagonize multiple GAG-dependent growth factors such as HGF, FGF2, and VEGF by blocking their ability to interact with GAGs on the cell surface (Merkulova-Rainon et al., (2003) J Biol Chem. 278:37400).

ii. Kringle Domains

HGF contains four kringle domains designated K1, K2, K3, and K4. Based on the exemplary amino acid sequence of HGF set forth in SEQ ID NO:3, the K1 domain includes amino acids 128 to 206, the K2 domain includes amino acids 211 to 288, the K3 domain includes amino acids 305 to 383, and the K4 domain includes amino acids 391 to 469. Participation of kringle domains in protein-protein interactions suggests the receptor binding site of HGF is localized within one or more of its kringle domains. Reduction of HGF activity by mutagenesis of the K1 domain of HGF indirectly supports a role of K1 in binding MET. Other studies showing that the K1 domain can mimic HGF activity directly demonstrates a functional K1/MET interaction. For example, the K1 domain alone, but not the N-terminal domain, is sufficient to bind to and activate the MET receptor, such as by induction of receptor tyrosine kinase activation, MAP kinase activation, cell motility and cell proliferation. K1-mediated functions are heparin dependent and heparin independent: for example, K1 stimulation of mitogenic signaling is heparin dependent while K1 stimulation of cell motility is heparin independent (Rubin et al., (2001) J Biol Chem. 276: 32977). The K1 domain itself does not bind heparin suggesting that heparin sulfate may facilitate K1 signaling through a mechanism other than HGF-heparin sulfate binding such as direct interaction of heparin sulfate with the MET receptor. Indeed, other growth factor ligand/receptors require heparin binding for function. For example, FGF signaling through the FGFR requires not only FGF-heparin sulfate binding but also an interaction between FGFR and heparin sulfate. In support of this, the extracellular domain of MET contains a heparin binding site. Thus, the conflicting requirements of heparin sulfate for mediating K1-induced motogenic and mitogenic responses suggests that MET-heparin sulfate interactions recruit intracellular effectors that mediate distinct cellular responses. The reduced potency of recombinant K1 in stimulating DNA synthesis and cell motility compared to full length HGF or an isoform of HGF (NK1, see below), suggests that HGF containing an N-terminal domain that can bind heparin sulfate modulates self-association of the ligand thereby potentiating HGF signaling.

Generally, kringle domains also are associated with angiogenesis inhibition due to their protein binding ability, as evidenced by a number of proteins containing kringle domains. For example, angiostatin (a molecule containing the K1-K4 domains of plasminogen) inhibits the proliferation and migration of endothelial cells, and induces apoptosis. Similarly, the K2 domain of Prothrombin, the K1-K2 domains of tPA, and the K1 domain of uPA all demonstrate anti-angiogenic properties. The mechanism for inhibition of angiogenesis by kringle domains is postulated to involve interactions with putative angiogenic binding molecules on endothelial cells, such as for example, binding to ATP synthase, angiomotin, αvβ3 integrin, annexin II, or any one or more growth factor receptors such as MET (Matsumoto et al., (2005) Biochem Biophys Res Commun. 333:316; Kuba et al., (2000) Biochem. Biophys. Res. Commun. 279:846). As such, the K1-K4 domains, in the absence of the N-terminal domain or β-chain of HGF, are sufficient to mediate angioinhibitory activities of HGF (Kuba et al., (2000) Biochem. Biophys. Res. Commun. 279:846). The K1 domain also functions independently to inhibit growth factor-induced angiogenic functions, such as endothelial cell proliferation stimulated by FGF2 (Xin et al., (2000) Biochem. Biophys. Res. Commun. 277:186). The K3 and K4 domains in combination with the first two kringle domains display anti-angiogenic properties as discussed above, and also are important in facilitating interactions with the β-chain that are necessary for receptor activation (see below).

iii. β-Chain

The β-chain of HGF, containing amino acids 495 to 728 of the exemplary HGF set forth in SEQ ID NO:3, structurally resembles a serine protease and contains a serine protease (SerP) domain but lacks proteolytic activity due to two nonconservative substitutions within the catalytic triad. The β-chain of HGF is unable to bind to the MET receptor and alone exhibits none of the activities of HGF. Deletion, however, of the β-chain results in loss of biological activity of HGF even though the α-chain alone can bind to the HGF receptor (Date et al (1997) FEBS Letters 420:1-6). Concomitant stimulation of cells with a recombinant molecule containing essentially the α-chain containing all four kringle domains of HGF (NK4 isoform, see below) and the β-chain of HGF together induce HGF responses (Matsumoto et al., (1998) J Biol Chem. 36:22913). Administration of the β-chain with an HGF isoform containing only the N-terminal domain and two kringle domains does not support receptor binding or receptor activation by the β-chain. These results suggest a cooperative interaction between the α and β chains that is dependent on the presence of the K3 and K4 domains of HGF for interaction with the β chain. Thus, the β chain of HGF is required for optimum activation and subsequent activation of intracellular signal transduction pathways that lead to HGF-mediated mitogenic, morphogenic, and motogenic responses.

2. HGF Variants

a. HGF Splice Variants

In addition to the full-length isoform of HGF, at least three additional splice variants of HGF have been identified in vivo. One, referred to as deleted HGF (delHGF, SEQ ID NO:24), contains a 5 amino acid deletion in the first kringle domain. delHGF shows similar activities to the full length HGF. The other two natural variants of HGF, termed NK1 (SEQ ID NO:30) and NK2 (SEQ ID NO:22), contain the N-terminal N domain followed by the first kringle domain (NK1) or the first two kringle domains (NK2). NK1 and NK2 display many of the functions of full-length HGF, however, experimental studies propose antagonist and agonist roles for these HGF isoforms. An engineered variant of HGF, termed NK4, contains the N-terminal N domain and all four kringle domains and functions as an antagonist of HGF since it can compete with full-length HGF for binding to MET, but it unable to stimulate detectable phosphorylation of the receptor. Other proposed isoforms of HGF include those set forth in SEQ ID NOS: 26 or 28.

The agonist or antagonist activities of NK1 and NK2 are contextual and depend on the cell type and/or experimental conditions. In particular, the agonistic functions of these HGF isoforms are correlated with heparin binding ability. This is because there is an important difference in the mechanism of receptor binding and activation of HGF and the truncated HGF forms NK1 and NK2. Mature HGF is postulated to induce MET receptor dimerization by forming a dimeric ligand and/or inducing a conformational change in the receptor tyrosine kinase, whereas NK1 and NK2 alone are unable to do this because they exist as monomers. The presence of heparin or GAGs can promote ligand dimerization and/or ligand-receptor oligomerization of some growth factor ligands. This allows a monomeric growth factor to induce receptor dimerization which is required for receptor activation. The crystal structure of NK1 predicts a model whereby repeating units of heparin sulfate bind two NK1 molecules through interaction with their respective N-terminal domains, thereby facilitating ligand dimerization and transactivation of the associated receptor kinases (Rubin et al., (2001) J Biol Chem., 276:32977). Thus, HGF is fully active in cells lacking heparin sulfate while NK1 and NK2 are only active in the presence of heparin or in cells that display heparin sulfate. Both NK1 and NK2 retain the N-terminal domain which mediates binding to heparin or the closely related heparin sulfate glycosaminoglycan (HSGAG) on the surface of cells. For example, in cells that lack heparin (i.e. heparin sulfate (HS)-deficient CHO cells) NK1 is unable to bind to MET. In contrast, heparin expressing cells or the addition of heparin to heparin-deficient cells exhibit ligand binding of the HGF isoforms and increased ligand-dependent activation of MET (Sakata et al., (1997) J Biol Chem. 272: 9457). Further, NK1 and NK2 retain proliferative activity in the presence, but not the absence of heparin (Schwall et al., (1996) J. Cell Biol. 133: 709-718). Thus, in the presence of heparin, or in the presence of heparin-expressing cells, NK1 and NK2 can function as agonists with properties very similar to HGF, but in the absence of heparin they can functions as antagonists. Importantly, depending on the cell type used and the presence of heparin, NK1 and NK2 can function either as an agonist or antagonist.

In vivo studies of the activities of NK1 and NK2 using transgenic mice demonstrate that the functions of NK1 and NK2 are distinct. NK1 transgenic mice exhibit a phenotype similar to HGF transgenic mice suggesting that NK1 indeed is a partial agonist and retains the ability to bind and activate the receptor in vivo (Jakubczak et al., (1998) Mol. Cell. Biol. 18: 1275). In contrast, mice transgenic for NK2 exhibit a dissociated agonist and antagonist phenotype. NK2 transgenic mice display agonist activity with respect to motogenic properties of MET-driven metastatic dissemination, but display antagonist mitogenic activity compared to the dysregulated cell growth observed in HGF and NK1 transgenic mice (Otsuka et al. (2000) Mol Cell Biol. 20: 2055).

NK4 (SEQ ID NO: 32), an engineered variant of HGF, is a true antagonist of HGF. NK4 antagonizes the mitogenic, motogenic, morphogenic, and tumor inhibitory activities of HGF. NK4 is prepared by enzymatic digestion of a highly purified recombinant HGF with elastase. Digestion of HGF with elastase yields two fragments; a fragment composed of the N-terminal 447 amino acids of the α-chain, including the N-terminal hairpin domain and four kringle domains (termed NK4), and a second fragment containing the β-chain and a portion of the α-chain containing the ⁴⁸⁷Cys which forms a disulfide bridge with the β-chain. NK4 binds to MET, although with reduced affinity compared to HGF, but it unable to activate the receptor due to the absence of the β-chain. Unlike other HGF isoforms, including for example NK1 or NK2, the presence of K4 in NK4 may induce a conformational change in the HGF thereby inhibiting receptor dimerization and activation, unless the β-chain is present (Matsumoto et al., (1998) J Biol Chem., 36:22913). Thus, NK4 competitively competes for HGF binding to the MET receptor and thereby antagonizes the biological functions of HGF. For example, NK4 inhibits HGF-induced cellular migration, invasion, and adhesion of cancer cells including breast, bladder, colorectal cancer cells, prostate, glioma, pancreatic, gastric, lung, and ovarian cancer cells (Jiang et al., (2005) Crit Rev Onc. Hema. 53:35). NK4 also inhibits other functions of HGF including HGF-induced vascular tubule formation from endothelial cells (Jiang et al., (1999) Clin Cancer Res 5:3695) and disruption of cell adhesion and tight junctions mediated by HGF signaling (Martin et al., (2004) Cell Biol Int 28: 361). Deletion of the N-terminal domain from NK4 abrogates the NK4-mediated HGF-antagonist activity demonstrating that the N-terminal domain is critical for binding of NK4 to MET (Kuba et al., (2000) Biochem. Biophys. Res. Commun. 279:846).

Besides acting as an antagonist of HGF, NK4 also displays general anti-angiogenic properties. The anti-angiogenic properties of NK4, including inhibition of proliferation and migration of endothelial cells, is independent of the MET receptor since NK4 antagonizes not only HGF- but FGF-2- and VEGF-mediated functions. The kringle domains of HGF are associated with angiogenesis inhibition (Kuba et al., (2000) Biochem. Biophys. Res. Commun. 279:846), and in fact, the K1 domain of HGF has been implicated in the angioinhibitory activity of NK4 since the first kringle domain alone is sufficient to inhibit cell proliferation stimulated by FGF-2 and enhance apoptosis in bovine aortic endothelial cells (Xin et al., (2000) Biochem. Biophys. Res. Commun. 277:186). The N-terminal domain also displays some anti-angiogenic function as it competes with growth factors, such as for example HGF, FGF-2, and VEGF, for binding to heparin (Merkulova-Rainon et al., (2003) J Biol Chem. 278:37400). Thus, the kringle domains, particularly K1, are responsible for the angioinhibitory activity of NK4, while the N-terminal domain of HGF augments the anti-angiogenic activities through competitive inhibition of binding of angiogenic growth factors to endothelial cells (Matsumoto et al., (2005) Biochem. Biophys. Res. Commun. 333:316). NK4 is postulated as a broad anti-cancer therapeutic candidate due to its bifunctionality as an HGF-antagonist and general angiogenesis inhibitor, mediating diverse anti-tumor activities such as inhibition of tumor metastasis, inhibition of invasion, inhibition of extracellular matrix degradation, and inhibition of tumor angiogenesis (Matsumoto et al., (2005) Biochem. Biophys. Res. Commun. 333:316).

b. HGF Allelic Variants

Variation occurs among members of a population or species (allelic variation) and also between species (species variation). An allelic variant of HGF can contain one or more nucleotide changes compared to SEQ ID NO: 1 or 2 or one or more amino acid changes compared to SEQ ID NO:3. Allelic variation can occur in any one or more of the exon or intron sequences of an HGF gene. Nucleic acids encoding HGF proteins and the encoded HGF polypeptides can include allelic variants of HGF. Exemplary allelic variants of HGF are set forth in Table 3. An exemplary HGF allelic variant can include any one or more nucleotide changes as set forth in SEQ ID NO: 15 or any one or more amino acid changes as set forth in SEQ ID NO:16.

In one example, allelic variants in HGF can include any one or more amino acid changes compared to a cognate HGF set forth in SEQ ID NO:3. For example, one or more amino acid variations can occur in the N-terminal domain of HGF. An allelic variant can include amino acid changes at position 78 where, for example, K can be replaced by N, or an amino acid change at position 82 where, for example, F can be replaced by L. An allelic variant of HGF also can occur in any one of the kringle domains of HGF. For example, an allelic variant can include amino acid changes in the K1 domain, such as an amino acid change at position 153 where, for example, S can be replaced by I, or at position 180 where, for example, P can be replaced by T. Additional amino acid changes can occur in the K3 domain. An allelic variant can include an amino acid change at position 293 where, for example, M can be replaced by V, or at position 300 where, for example, L can be replaced by M, or at position 304 where, for example, E can be replaced by K, or at position 317 where, for example, V can be replaced by A, or at position 325 where, for example, P can be replaced by S, or at position 330 where, for example D can be replaced by Y, or at position 336 where, for example, E can be replaced by K. Allelic variants also can occur in the K4 domain such as an amino acid change at position 387 where, for example, H can be replaced by N, or at position 416 where, for example, D can be replaced by N. Other allelic variations can occur in the serine protease domain of HGF. An allelic variant can include an amino acid change at position 494 where, for example, R can be replaced by Q, or at position 505 where, for example, I can be replaced by V, or at position 509 where, for example, V can be replaced by I, or at position 558 where, for example, D can be replaced by E, or at position 561 where, for example, C can be replaced by R, or at position 592 where, for example, D can be replaced by N, or at position 595 where, for example, S can be replaced by N.

In some cases, a nucleotide or amino acid difference can be “silent”, having no or virtually no detectable effect on a biological activity. In other examples, an allelic variant can result in a truncated or shortened polypeptide. For example, an allelic variation at nucleotide position 1256 where for example, G can be replaced by N, results in a change to a stop codon resulting in a translated protein that is shortened. In other examples, allelic variants, for example in the context of a wildtype or predominant form of the ligand, can be associated with a disease, condition, or change in biological activity.

TABLE 3

| | | Nucleotide | |
|---|---|---|---|
| Polymorphism | SNP # | change | Amino acid change |
| NT: 293 | 17855203 | 293 A/G | none |
| NT: 409 | | 409 T/C | AA 82 F/L |
| NT: 498 | 5745635 | 498 A/G | none |
| NT: 623 | 17566 | 623 G/T | AA 153 S/I |
| NT: 876 | 5745666 | 876 T/C | none |
| NT: 1075 | 5745687 | 1075 G/A | AA 304 E/K |
| NT: 1138 | | 1138 C/T | AA 325 P/S |
| NT: 1153 | 5745688 | 1153 G/T | AA 330 D/Y |
| NT: 1256 | 5745703 | 1256 G/A | Stop |
| AA: 494 | | | AA 494 R/Q |
| AA: 78 | | | AA 78 K/N |
| AA: 180 | | | AA 180 P/T |
| AA: 293 | | | AA 293 M/V |
| AA: 300 | | | AA 300 L/M |
| AA: 317 | | | AA 317 V/A |
| AA: 336 | | | AA 336 E/K |
| AA: 387 | | | AA 387 H/N |
| AA: 416 | | | AA 416 D/N |
| AA: 505 | | | AA 505 I/V |
| AA: 509 | | | AA 509 V/I |
| AA: 558 | | | AA 558 D/E |
| AA: 561 | | | AA 561 C/R |
| AA: 592 | | | AA 592 D/N |
| AA: 595 | | | AA 595 S/N |

Variants of HGF also include species variants. HGF is present in multiple species besides human such as, but not limited to, cow, dog, cat, mice, rats, and chicken. Exemplary sequences for species variants of HGF are set forth in any one of SEQ ID NOS:246-251.

3. MET Receptor

MET receptor (also called c-MET, hepatocyte growth factor receptor, HGFR) is a receptor tyrosine kinase (RTK) that is produced as a precursor protein that is proteolytically cleaved into a heterodimeric molecule composed of an extracellular 50-kDa α chain disulfide-linked to a transmembrane 145-kDa β chain. In the fully processed MET protein, the α subunit contains a Sema domain involved in protein-protein interactions, and a cysteine-rich motif called a MET-related sequence. The β subunit, which traverses the membrane and is extracellular and intracellular, contains three functional domains including a juxtamembrane domain, the catalytic domain, and the C-terminal tail. HGF is the ligand for MET. Binding of HGF to MET triggers receptor dimerization and phosphorylation on multiple residues within the juxtamembrane, catalytic, and cytoplasmic tail domains, thereby regulating receptor internalization, catalytic activity, and multi-substrate docking. For example, the juxtamembrane domain contains a Ser985 residue that upon serine phosphorylation inhibits the kinase activity of MET; dephosphorylation of Ser985 allows HGF-dependent MET activation. The juxtamembrane domain also contains Tyr1003 that participates in the negative regulation of MET by targeting MET for ubiquitination and degradation by the proteasome pathway. The phosphorylation sites within the catalytic domain of MET include Tyr1230, Tyr1234, and Tyr1235 and the phosphorylation sites within the cytoplasmic tail include Tyr1349 and Tyr1356. Phosphorylation and activation of MET results in binding and/or phosphorylation of many intracellular signaling proteins including multiple adaptor proteins (e.g., Grb2, Shc, Cbl, Crk, cortectin, paxillin, and GAB 1), and a variety of other signal transducers (e.g., PI 3-kinase, FAK, Src, ERK 1/2, JNK 1/2, PLC-gamma, and STAT-3.

MET is highly expressed in epithelial cells and hepatocytes, but also is expressed on other cells of hematopoietic origin including germinal center B cells and terminally differentiated plasma cells. MET also is expressed in many cancer tissues and on solid tumors. Normally, HGF-MET signaling is involved in embryonic development, although MET signaling also mediates growth, invasion, motility, and metastasis of cancer cells as well as promotes angiogenesis in tumors. In addition to a role in cancer, MET also is a critical factor in the development of malaria infection as a mediator of signals that makes the host susceptible to infection, such as by rearranging the host-cell actin cytoskeleton and inhibiting apoptosis of infected cells (Carrolo et al., (2003) Nat Med., 9:1363; Leiriao et al., (2005) Cell Microbiol. 7:603).

An exemplary MET receptor (GenBank No. NP_{—}000236 set forth as SEQ ID NO:34) contains of an α chain between amino acids 1-307 and a β chain between amino acids 308-1390, with the intracellular domain of the β chain between residues 956-1390. MET is characterized by a Sema domain, between amino acids 55-500. In MET, the Sema domain is involved in receptor dimerization in addition to ligand binding. The MET protein also is characterized by a plexin cysteine rich repeat between amino acids 519-562 and three IPT/TIG domains between amino acids 563-655, amino acids 657-739 and amino acids 742-836. IPT stands for Immunoglobulin-like fold shared by Plexins and Transcription factors. TIG stands for the Immunoglobulin-like domain in transcription factors (Transcription factor IG). TIG domains in MET likely play a role in mediating some of the interactions between extracellular matrix and receptor signaling. The MET protein also is characterized by a transmembrane domain between amino acids 933-955, followed by a juxtamembrane domain beginning at amino acid 956, a cytoplasmic protein kinase domain between amino acids 1078-1337, and a cytoplasmic tail.

### C. HGF Isoforms

Provided herein are HGF isoforms and methods of using HGF isoforms for modulating mitogenesis, morphogenesis, and angiogenesis, including via MET receptor activities. In one embodiment, the HGF isoforms provided herein differ from the full-length HGF cognate ligand in that the nucleic acids encoding the isoforms retain part or all of any one or more of the seventeen introns. The resultant HGF isoform polypeptides contain insertions and/or deletions of amino acids such that the HGF protein includes a disruption or elimination of all of or a portion of one or more domains of a cognate HGF and thereby exhibit a difference in one or more activities or functions or structure compared to the cognate ligand. For example, the changes that HGF isoforms exhibit compared to an HGF can include, but are not limited to, elimination and/or disruption of all or part of a signal peptide, an N-terminal domain, one or more Kringle domains and/or a Ser-P domain. The HGF isoforms provided herein can be used for modulating the activity of a cell surface receptor, including a MET receptor, a VEGFR or a FGFR. They also can be used as targeting agents for delivery of molecules, such as drugs or toxins or nucleic acids, to targeted cells or tissues in vivo or in vitro.

Pharmaceutical compositions containing one or more HGF isoforms, typically one or more different isoforms, are provided. The pharmaceutical compositions can be used to treat diseases that include cancers, other diseases that manifest aberrant angiogenesis, malaria, and other diseases known to those of skill in the art in which a MET or angiogenic receptor such as a MET, VEGFR, or FGFR, are implicated, involved or in which they participate. Cancers include breast, lung, colon, gallbladder, gastric, pancreatic, mammary, ovarian, and prostate cancers, glioblastoma, lymphoma, malignant melanoma, and others.

Also provided are methods of treatment of diseases and conditions by administering the pharmaceutical compositions or delivering a HGF isoform, such by administering a vector that encodes the isoform. Administration can be effected in vivo or ex vivo.

Methods are provided herein for producing, isolating and formulating HGF isoforms, including producing HGF isoforms and nucleic acid molecules encoding HGF isoforms. Also provided are combinations of HGF isoforms with other modulators of MET signaling.

1. Classes of HGF Isoforms

As noted, HGF isoforms are polypeptides that lack a domain or portion of a domain or have a disruption of a domain compared with a wildtype or predominant form of HGF sufficient to remove or reduce or otherwise alter, including having a positive or negative effect on, an activity compared to the cognate ligand. HGF isoforms represent splice variants of an HGF gene (or recombinant shortened variants) that can be generated by alternate splicing or by recombinant or synthetic methods. HGF isoforms can be encoded by alternatively spliced RNAs. HGF isoforms also can be generated by recombinant methods and by use of in silico and synthetic methods.

Typically, an HGF isoform produced from an alternatively spliced RNA is not a predominant form of a polypeptide encoded by a gene. In some instances, an HGF isoform can be a tissue-specific or developmental stage-specific polypeptide or can be disease specific (i.e., can be expressed at a different level from tissue-to-tissue or stage-to-stage or in a diseased state compared to a non-diseased state or only may be expressed in the tissue, at the stage, or during the disease process or progress). Alternatively spliced RNA forms that can encode HGF isoforms include, but are not limited to, exon deletion, exon retention, exon extension, exon truncation, and intron retention alternatively spliced RNAs. Included among HGF isoforms are intron fusion proteins.

2. Alternative Splicing and Generation of HGF Isoforms

Genes in eukaryotes include intron and exons that are transcribed by RNA polymerase into RNA products generally referred to as pre-mRNA. Pre-mRNAs are typically intermediate products that are further processed through RNA splicing and processing to generate a final messenger RNA (mRNA). Typically, a final mRNA contains exon sequences and is obtained by splicing out the introns. Boundaries of introns and exons are marked by splice junctions, sequences of nucleotides that are used by the splicing machinery of the cell as signals and substrates for removing introns and joining together exon sequences. Exons are operatively linked together to form a mature RNA molecule. Typically, one or more exons in an mRNA contains an open reading frame encoding a polypeptide. In many cases, an open reading frame can be generated by operatively linking two or more exons; for example, a coding sequence can span exon junctions and an open reading frame is maintained across the junctions.

RNA also can undergo alternative splicing to produce a variety of different mRNA transcripts from a single gene. Alternatively spliced mRNAs can contain different numbers of and/or arrangements of exons. For example, a gene that has 10 exons can generate a variety of alternatively spliced mRNAs. Some mRNAs can contain all 10 exons, some with only 9, 8, 7, 6, 5 etc. In addition, products for example, with 9 of the 10 exons, can be among a variety of mRNAs, each with a different exon missing. Alternatively spliced mRNAs can contain additional exons, not typically present in an RNA encoding a predominant or wild type form. Addition and deletion of exons includes addition and deletion, respectively, of a 5′ exon, 3′exon and an exon internal in an RNA. Alternatively spliced RNA molecules also include addition of an intron or a portion of an intron operatively linked to or within an RNA. For example, an intron normally removed by splicing in an RNA encoding a wildtype or predominant form can be present in an alternatively spliced RNA. An intron or intron portion can be operatively linked within an RNA, such as between two exons. An intron or intron portion can be operatively linked at one end of an RNA, such as at the 3′ end of a transcript. In some examples, the presence of an intron sequence within an RNA terminates transcription based on poly-adenylation sequences within an intron.

Alternative RNA splicing patterns can vary depending upon the cell and tissue type. Alternative RNA splicing also can be regulated by developmental stage of an organism, cell or tissue type. For example, RNA splicing enzymes and polypeptides that regulate RNA splicing can be present at different concentrations in particular cell and tissue types and at particular stages of development. In some cases, a particular enzyme or regulatory polypeptide can be absent from a particular cell or tissue type or at particular stage of development. These differences can produce different splicing patterns for an RNA within a cell or tissue type or stage, thus giving rise to different populations of mRNAs. Such complexity can generate a number of protein products appropriate for particular cell types or developmental stages.

Alternatively spliced mRNAs can generate a variety of different polypeptides, also referred to herein as isoforms. Such isoforms can include polypeptides with deletions, additions and shortenings. For example, a portion of an open reading frame normally encoded by an exon can be removed in an alternatively spliced mRNA, thus resulting in a shorter polypeptide. An isoform can have amino acids removed at the N or C terminus or the deletion can be internal. An isoform can be missing a domain or a portion of a domain as a result of a deleted exon. Alternatively spliced mRNAs also can generate polypeptides with additional sequences. For example, a stop codon can be contained in an exon; when this exon is not included in an mRNA, the stop codon is not present and the open reading frame continues into the sequences contained in downstream exons. In such example, additional open reading frame sequences add additional amino acid residues to a polypeptide and can result in the addition of a new domain or a portion thereof.

a. Intron Modification and Intron Fusion Proteins

Among the HGF isoforms that can be generated by alternative RNA splicing patterns are isoforms generated through intron modification, also called intron fusion proteins. In one example, an HGF isoform is generated by alternative splicing such that one or more introns are retained compared to an mRNA transcript encoding a wildtype or predominant form of HGF. The incorporated intron sequences can include one or more introns or a portion thereof. Such mRNAs can arise by a mechanism of intron retention. For example, a pre-mRNA is exported from the nucleus to the cytoplasm of the cell before the splicing machinery has removed one or more introns. In some cases, splice sites can be actively blocked, for example by cellular proteins, preventing splicing of one or more introns.

The retention of one or more intron sequences can generate transcripts encoding HGF isoforms that are shortened compared to a wildtype or predominant form of HGF. A retained intron sequence can introduce a stop codon in the transcript and thus prematurely terminate the encoded polypeptide. A retained intron sequence also can introduce additional amino acids into an HGF polypeptide, such as the insertion of one or more codons into a transcript such that one or more amino acids are inserted into a domain of HGF. Intron retention includes the inclusion of a full or partial intron sequence into a transcript encoding an HGF isoform. The retained intron sequence can introduce nucleotide sequence with codons in-frame to the surrounding exons or it can introduce a frame shift into the transcript. Exemplary nucleotide sequences of intron retention transcripts include SEQ ID NOS:9, 11, or 13.

Generally, an intron fusion protein is an isoform that, due to the retention of any one or more intron sequences, lacks a domain or portion of a domain or contains an additional domain or portion of a domain sufficient to alter a biological activity compared to a cognate ligand. In addition, an intron fusion protein can contain one or more amino acids not encoded by an exon, operatively linked to exon-encoded amino acids resulting in an isoform that is lengthened or shortened compared to a wildtype or predominant form encoded by an HGF gene. Typically, an intron fusion protein is shortened by the presence of one or more stop codons in an intron fusion protein-encoding RNA that are not present in the corresponding sequence of an RNA encoding a wildtype or predominant form of an HGF polypeptide. Addition of amino acids and/or a stop codon can result in an intron fusion protein that differs in size and sequence from a wildtype or predominant form of a polypeptide.

An intron fusion protein can be modified in one or more biological activities. For example, addition of amino acids in an intron fusion protein can add, extend or modify a biological activity compared to a wildtype or predominant form of a polypeptide. For example, fusion of an intron encoded amino acid sequence to a protein can result in the addition of a domain with new functionality. Fusion of an intron encoded polypeptide to a protein also can modulate an existing biological activity of a protein, such as by inhibiting a biological activity, for example, inhibition of receptor dimerization and/or inhibition of receptor signaling.

Intron fusion proteins include natural and combinatorial intron fusion proteins. A natural intron fusion protein is encoded by an alternatively spliced RNA that contains one or more introns or a portion thereof operatively linked to one or more exons of a gene. Combinatorial intron fusion proteins are generated by recombinant or synthetic means and often mimic a natural intron fusion protein in that an intron-encoded sequence can be operatively linked to exon sequence(s) thereby encoding a polypeptide where one or more domains or a portion thereof is/are deleted or added as in a natural intron fusion protein derived from the same gene sequence or derived from a gene sequence in a related gene family.

i. Natural Intron Fusion Proteins

Natural intron fusion proteins are generated from a class of alternatively spliced mRNAs that include mRNAs containing intron sequence as well as exon sequences, such as intron retention RNA molecules and some exon extension RNAs. They include all such variants that occur and can be isolated from a cell or tissue or identified in a database. Any splice variant that is possible and that includes one or more codons (including only a stop codon) from an intron is considered a natural intron fusion protein.

Retention of one or more introns or a portion thereof can lead to the generation of isoforms referred to herein as natural intron fusion proteins. For example, an intron sequence can contain an open reading frame that is operatively linked to the exon sequences by RNA splicing. Intron-encoded sequences can add amino acids to a polypeptide, for example, at either the N- or C-terminus of a polypeptide, or internally within a polypeptide. In some examples, an intron sequence also can contain one or more stop codons. An intron encoded stop codon that is operatively linked with an open reading frame in one or more exons can terminate the encoded polypeptide. Thus, an isoform can be produced that is shortened as a result of the stop codon. In some examples, an intron retained in an mRNA can result in the addition of one or more amino acids and a stop codon to an open reading frame, thereby producing an isoform that terminates with an intron encoded sequence.

Provided herein are natural intron fusion proteins, that can be generated by intron retention, including intron fusion proteins with addition of domains or portion of domains encoded by an intron, and intron fusion proteins with one or more domains or portion of domain deleted. For example, an intron sequence can be operatively linked in place of an exon sequence that is typically within an mRNA for a gene. A domain or portion thereof encoded by the exon is thus deleted and intron encoded amino acids are included in the encoded polypeptide.

In another example, an intron sequence is operatively linked in addition to the typically present exons in an mRNA. In one example, an operatively linked intron sequence can introduce a stop codon in-frame with exon sequences encoding a polypeptide. In another example, an operatively linked intron sequence can introduce one or more amino acids into a polypeptide. In some embodiments, a stop codon in-frame also is operatively linked with exon sequences encoding a polypeptide, thereby generating an mRNA encoding a polypeptide with intron-encoded amino acids at the C-terminus.

In one example of a natural intron fusion protein, one or more amino acids encoded by an intron sequence are operatively linked at the C-terminus of a polypeptide. For example, an intron fusion protein is generated from a nucleic acid sequence that contains one or more exon sequences at the 5′ end of an RNA followed by one or more intron sequences or a portion of an intron sequence retained at the 3′ end of an RNA. An intron fusion protein produced from such nucleic acid contains exon-encoded amino acids at the N-terminus and one or more amino acids encoded by an intron sequence at the C-terminus. In another example, an intron fusion protein is generated from a nucleic acid by operatively linking a stop codon encoded within an intron sequence to one or more exon sequences, thereby generating a nucleic acid sequence encoding a shortened polypeptide.

ii. Combinatorial Intron Fusion Proteins

Intron fusion proteins also can be generated by recombinant methods and/or in silico and synthetic methods to produce polypeptides that are modified compared to a wildtype or predominant form of a polypeptide. Typically, such HGF isoforms have a modified sequence compared to a wildtype or predominant form due to the presence of an intron sequence operatively linked to an exon sequence of a gene. For example, as is described further herein, by using available software programs, intron and exons, sequences, and encoded protein domains can be identified in a nucleic acid, such as an HGF gene. Recombinant nucleic acid molecules encoding polypeptides can be synthesized that contain one or more exons and an intron sequence or portion thereof. Such recombinant molecules can contain 1, 2, 3, 4, 5, 6, 7, 8, 9, 10, or more amino acids and/or a stop codon encoded by an intron, operatively linked to an exon, producing an intron fusion protein.

An intron fusion protein generated by recombinant means can include a polypeptide that is longer or shorter compared to a wildtype or predominant form due to the presence of the encoded intron sequence. Typically, combinatorial intron fusion proteins are shortened polypeptides compared to a wildtype or predominant form. For example, recombinant molecules can contain one or more amino acids and/or a stop codon encoded by an intron, operatively linked to an exon, producing an isoform that is shorter than a wildtype or predominant form of HGF. Shortening can remove one or more domains or a portion thereof. These truncated forms can have deletions internally, at the N-terminus, at the C-terminus or a combination thereof. In another example, an intron sequence can result in a lengthened protein if the intron-encoded amino acid sequence results in the introduction of additional amino acids into an HGF polypeptide, such as the insertion of one or more codons into a transcript such that one or more amino acids are inserted into a domain of HGF, or result in the addition of a domain. Alternatively, an encoded intron sequence can result in a frame shift of an HGF transcript such that a stop codon is not read in a downstream exon resulting in a lengthened transcript. As part of this method, potential immunogenic epitopes can be recognized using motif scanning, and modified with conservative amino acid substitutions or by other modifications well known in the art, such as pegylation. Generally, any therapeutic intron fusion protein can be modified in this same way to achieve optimized pharmacokinetics or avoid immunogenicity.

b. Isoforms Generated by Exon Modifications

HGF isoforms also can be generated by modification of an exon relative to a corresponding exon of an RNA encoding a wildtype or predominant form of a HGF polypeptide. Exon modifications include alternatively spliced RNA forms such as exon truncations, exon extensions, exon deletions and exon insertions. These alternatively spliced RNA molecules can encode HGF isoforms which differ from a wildtype or predominant form of a HGF polypeptide by including additional amino acids and/or by lacking amino acid residues present in a wildtype or predominant form of a HGF polypeptide.

An inserted exon can operatively link additional amino acids encoded by the inserted exon to the other exons present in an RNA. An inserted exon also can contain one or more stop codons such that the RNA encoded polypeptide terminates as a result of such stop codons. If an exon containing such stop codons is inserted upstream of an exon that contains the stop codon used for polypeptide termination of a wildtype or predominant form of a polypeptide, a shortened polypeptide can be produced.

An inserted exon can maintain an open reading frame, such that when the exon is inserted, the RNA encodes an isoform containing an amino acid sequence of a wildtype or predominant form of a polypeptide with additional amino acids encoded by the inserted exon. An inserted exon can be inserted 5′, 3′ or internally in an RNA, such that additional amino acids encoded by the inserted exon are linked at the N terminus, C-terminus or internally, respectively in an isoform. An inserted exon also can change the reading frame of an RNA in which it is inserted, such that an isoform is produced that contains only a portion of the sequence of amino acids in a wildtype or predominant form of a polypeptide. Such isoforms can additionally contain amino acid sequences encoded by the inserted exon and also can terminate as a result of a stop codon contained in the inserted exon.

HGF isoforms also can be produced from exon deletion events. Deletion of an exon can produce a polypeptide of alternate size such as by removing sequences that encode amino acids as well as by changing the reading frame of an RNA encoding a polypeptide. An exon deletion can remove one or more amino acids from an encoded polypeptide; such amino acids can be N-terminal, C-terminal or internal to a polypeptide depending upon the location of the exon in an RNA sequence. Deletion of an exon in an RNA also can cause a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide. In one example, a shift of reading frame produces an isoform that is shortened compared to a wildtype or predominant form of a polypeptide. Such shortened isoforms also can contain sequences of amino acids not present in a wildtype or predominant form of a polypeptide.

HGF isoforms also can be produced by exon extension in an RNA. Additional sequence contained in an exon extension can encode additional amino acids and/or can contain a stop codon that terminates a polypeptide. An exon insertion containing an in-frame stop codon can produce a shortened isoform, that terminates in the sequence of the exon extension. An exon insertion also can shift the reading frame of an RNA, resulting in an isoform containing one or more amino acids not present in a wildtype or predominant form of a polypeptide and/or an isoform that terminates at a sequence different from that of a wildtype or predominant form of a polypeptide. An exon extension can include sequences contained in an intron of an RNA encoding a wildtype or predominant form of a polypeptide and thereby produce an intron fusion protein.

HGF isoforms also can be produced by exon truncation. An RNA molecule with an exon truncation can produce a polypeptide that is shortened compared to a wildtype or predominant form of a polypeptide. An exon truncation also can result in a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide.

Alternatively spliced RNA molecules including exon modifications can produce HGF isoforms that a lack a domain or a portion thereof sufficient to reduce or remove a biological activity. For example, exon modified RNA molecules can encode shortened HGF polypeptides that lack a domain or portion thereof. Exon modified RNA molecules also can encode polypeptides where a domain is interrupted by inserted amino acids and/or by a shift in reading frame that interrupts a domain with one or more amino acids not present in a wildtype or predominant form of a polypeptide.

2. HGF Isoform Polypeptide Structure

The exemplary HGF gene (see e.g., SEQ ID NO:1, FIG. 1) includes 18 exons that contain a protein coding sequence interrupted by 17 introns. In a wildtype or predominant form of an HGF polypeptide, such as the polypeptide set forth in SEQ ID NO:3, which can be encoded by a nucleic acid molecule whose sequence is set forth in SEQ ID NO:2, 18 exons are joined by RNA splicing to form a transcript encoding a 728 amino acid polypeptide that includes a signal sequence, an N-terminal domain, four kringle domains (K1-K4), and a SerP domain (see. e.g, FIG. 2). HGF isoforms such as those provide herein, can be generated by alternative splicing such that the splicing pattern of the HGF is altered compared to the transcript encoding a wildtype or predominant form of HGF.

HGF isoforms generated by alternative splicing, such as by exon deletion, exon retention, exon extension, exon truncation, or intron retention, generally result in a ligand that lacks a domain or portion of a domain or that has a disruption in a domain such as by the insertion of one or more amino acids compared to HGF polypeptides of a wildtype or predominant form of the ligand. HGF isoforms also can contain a new domain and/or a function compared to a wildtype and/or predominant form of the ligand. The deletion, disruption and or insertion in the polypeptide sequence of an HGF isoform is sufficient to alter an activity compared to that of an HGF or change the structure compared to an HGF, such as by elimination of one or more domains or by addition of a domain or portion thereof, such as one encoded by an intron in the HGF gene. Provided herein are HGF isoforms generated by intron retention that lack all or some domains of an HGF polypeptide. HGF isoforms provided herein also can include intron-encoded amino acids that are inserted internally, or at the N- or C-terminus of an encoded isoform compared to a cognate ligand.

HGF isoforms can lack one or more domains or part of one or more domains compared to the polypeptide sequence of a wildtype or predominant form of the ligand. For example, an HGF isoform can lack the SerP domain or part of the SerP domain. Such isoforms can lack some or all of amino acids set forth as amino acids 495-728 of SEQ ID NO:3. Exemplary HGF isoforms lacking a SerP domain include SEQ ID NOS: 10, 12, 18, or 20 and exemplary HGF isoforms lacking some of a SerP domain include SEQ ID NO: 14. An HGF isoform can lack all or a part of a Kringle domain. Such isoforms include isoforms that lack any one or more or part of any one or more of the four Kringle domains including the K1, K2, K3, or K4 domain. An HGF isoform can lack part of the first Kringle domain, all of the first Kringle domain, part of the second Kringle domain, all of the second Kringle domain, part of the third Kringle domain, all of the third Kringle domain, part of the fourth Kringle domain, and/or all of the fourth Kringle domain, or combinations thereof. Such isoforms can lack some or all of amino acids set forth as amino acids 128-206 (K1), 211-288 (K2), 305-383 (K3), and/or 391-469 (K4) of SEQ ID NO:3. Exemplary HGF isoforms lacking part of a K1 domain include SEQ ID NO: 10 and 18. An HGF isoform also can lack all or part of an N-terminal domain.

An HGF isoform can include a disruption in a domain such as by the insertion of one or more amino acids compared to the polypeptide sequence of a wildtype or predominant form of HGF. For example, an HGF isoform can include an insertion of one or more amino acids in the signal peptide, in a N-terminal domain, in one or more of the Kringle domains, and/or in the SerP domain.

HGF isoforms also can include HGF polypeptide sequences that include the addition of a domain or a partial domain into the sequence. For example, an HGF isoform can include the addition of amino acids at the C-terminus of the protein, where such amino acid sequence is not found in the wildtype and/or predominant form of HGF. Exemplary HGF isoforms that include additional amino acid sequences at the C-terminal end of the polypeptide sequence include SEQ ID NOS: 10, 12, 18, or 20.

HGF isoform polypeptides also can contain amino acids that are not formally part of a domain but are found in between designated domains (referred to herein as linking regions). HGF isoforms also can include insertion, deletion and/or disruption in one or more linking regions. Exemplary HGF isoforms that include a disruption in a linking region include SEQ ID NOS: 10, 12, 18, or 20.

3. HGF Isoform Activities

The HGF isoforms provided herein can possess different or altered activities compared with a wildtype or predominant form of HGF. An HGF isoform can be an agonist, partial agonist, or antagonist of MET signaling. An HGF isoform also can exhibit other activities that are independent of HGF-MET signaling. Generally, an HGF isoform provided herein inhibits an activity of its receptor MET, such as by acting as a ligand antagonist. HGF isoforms, provided herein, also can inhibit angiogenic activities by other growth factor ligands including VEGF and FGF-2. Altered activities include, for example, altered signal transduction and/or altered interactions with one or more cell surface molecules.

Generally, an activity is altered by an isoform at least 0.1, 0.5, 1, 2, 3, 4, 5, or 10 fold compared to a wildtype and/or predominant form of the ligand. Typically, an activity is altered 10, 20, 50, 100 or 1000 fold or more. For example, an isoform can be reduced in an activity compared to a wildtype and/or predominant form of the ligand. An isoform also can be increased in an activity compared to a wildtype and/or predominant form of a ligand. In assessing an activity of an HGF isoform, the isoform can be compared with a wildtype and/or predominant form of HGF. For example, an HGF isoform can be altered in an activity compared to the HGF polypeptide set forth as SEQ ID NO:3. An isoform also can be tested for an antagonist or inhibitory activity by assessing an activity of an HGF isoform in the presence of a wildtype and/or predominant form of HGF, or in the presence of a wildtype and/or predominant form of other growth factors such as VEGF or FGF-2.

a. Cell Surface Action Alterations

In one example, an HGF isoform is altered in cell surface interaction, including receptor interaction. For example, an isoform is reduced in binding affinity for one or more receptors, such as for example a MET receptor. In another example, an isoform is increased in affinity for one or more receptors. An HGF isoform also can be altered in its binding to other cell surface molecules. In one example, isoforms can be altered in binding to GAGs, such as heparin or heparin sulfate. In another example, isoforms can be altered in binding to other cell surface proteins involved in angiogenesis, such as for example, endothelial ATP synthase, angiomotin, αvβ3 integrin, annexin II, any one or more growth factor receptors such as MET, FGFR, or VEGFR, or any other cell surface molecule known to cooperate with a growth factor receptor to induce angiogenic responses. An isoform also can be altered in specificity for a receptor or other cell surface binding molecule. For example, an isoform can bind one receptor or other cell surface protein preferentially over other receptors or cell surface proteins, where such preferential binding is in comparison to the receptor specificity of a wildtype or predominant form of HGF. Isoforms altered in receptor or cell surface interaction can include isoforms that lack all or part of a N-terminal domain or have a disruption of a N-terminal domain. HGF isoforms with altered receptor or cell surface binding also can include isoforms that lack all or part of any one or more of a K1, K2, K3, or K4 domain. HGF isoforms altered in receptor interaction also can include isoforms that have a conformational change compared to a wildtype or predominant form of HGF, including monomeric isoforms.

HGF isoforms altered in interaction with a cell surface molecule, including its receptor MET, can be altered in one or more facets of signal transduction. An isoform, compared with a wildtype or predominant form of HGF can be altered in the modulation of one or more cellular responses, including inducing, augmenting, suppressing and preventing cellular responses from a receptor or other cell surface protein, such as a protein involved in angiogenesis responses. Examples of cellular responses that can be altered by an HGF isoform, include, but are not limited to, induction of mitogenic, motogenic, morphogenic, and/or angiogenic responses.

b. Competitive Antagonist

An HGF isoform can compete with another HGF form, such as a wildtype or predominant form of a cognate HGF, for receptor binding. Such isoforms can thus bind the MET receptor and reduce the amount of receptor available to bind to other HGF polypeptides. HGF isoforms that bind and compete for one or more receptors of HGF can include HGF isoforms that do not participate in signal transduction or are reduced in their ability to participate in signal transduction compared to a cognate HGF.

An HGF isoform antagonist that competes with a predominant ligand by binding to the MET cell surface receptor can include an N-terminal domain and all or part of any one or more Kringle domains of a cognate HGF ligand. An antagonistic HGF isoform can lack one or more domains, such that the isoform although bound to its receptor does not modulate signal transduction. For example, such isoforms can lack all or part of a β-chain including all or part of a SerP domain. In one example, an HGF isoform lacks one or more amino acids of the SerP domain, for example, lacking one or more amino acids corresponding to amino acids 495-728 of the HGF polypeptide set forth as SEQ ID NO:3. An HGF isoform antagonist also can lack all or part of any one of the four kringle domains. In one example, an HGF isoform lacks one or more amino acids corresponding to any one of the kringle domains of the cognate ligand set forth as SEQ ID NO:3, such as one or more amino acids between amino acids 128-206 (K1), 211-288 (K2), 305-383 (K3), and/or 391-469 (K4) of SEQ ID NO:3.

C. Negatively Acting and Inhibitory Isoforms

HGF isoforms also can modulate an activity of another polypeptide. The modulated polypeptide can be a wildtype or predominant form of HGF or can be a wildtype or predominant form of another growth factor, such as, but not limited to, FGF-2 or VEGF. An HGF isoform also can modulate another HGF, FGF-2, or VEGF isoform, such as isoforms expressed in a disease or condition. Such HGF isoforms can act as negatively acting ligands by preventing or inhibiting one or more biological activities of a wildtype or predominant form of a growth factor ligand/receptor pair. An HGF isoform can interact directly or indirectly to modulate an activity of a HGF, or other growth factor polypeptide. A negatively acting ligand need not bind or affect the ligand binding domain of a receptor, nor affect ligand binding to the receptor.

In one example, an HGF isoform can compete with another growth factor ligand for binding to a cell surface protein necessary for mediating receptor dimerization and/or angiogenic responses of the growth factor. For example, an HGF isoform can compete with another growth factor ligand for binding to heparin or a GAG, thereby preventing the formation of a dimeric ligand required for ligand-mediated signaling of a cognate receptor. Such an HGF isoform includes all or part of an N-terminal domain of HGF sufficient to bind to a GAG. An HGF isoform further can lack all or part of any one or more of a K1, K2, K3, or K4 domain, or a SerP domain of a cognate HGF as long as the HGF isoform binds to a GAG but does not itself induce receptor dimerization and activation.

In another example, an HGF isoform can bind to a cell surface molecule that modulates or cooperates with the signaling induced by another ligand-receptor pair. For example, an HGF isoform can bind to a protein involved in the angiogenic response, such as for example endothelial ATP synthase, angiomotin, αvβ3 integrin, annexin II, a growth factor receptors such as MET, FGFR, or VEGFR, or any other cell surface molecule that modulates and/or cooperates with angiogenic signals induced by binding of HGF, VEGF, FGF-2, or other growth factor to its receptor. Such an HGF isoform includes all or part of a K1 domain. An HGF isoform further can lack all or part of any one or more of a N-terminal domain, K2, K3, K4, or SerP domain as long as the HGF isoform binds to an angiogenic molecule to modulate an angiogenic response induced by a growth factor, but does not itself induce MET receptor activation.

### D. Methods for Identifying and Generating HGF Isoforms

HGF isoforms can be identified and produced by any of a variety of methods. For example they can be identified by analysis and identification of genes and expression products (RNA molecules) using cloning methods in combination with bioinformatics methods such as sequence alignments and domain mapping and selections.

1. Methods for Identifying and Isolating Isoforms

Exemplary methods for identifying and isolating HGF isoforms include cloning of expressed gene sequences and alignment with a gene sequence such as a genomic DNA sequence. Expressed sequences, such as cDNA molecules or regions of cDNA molecules, are isolated. Primers can be designed to amplify a cDNA or a region of a cDNA. In one example, primers are designed which overlap or flank the start codon of the open reading frame of an HGF gene and primers are designed which overlap or flank the stop codon of the open reading frame. Primers can be used in PCR, such as in reverse transcriptase PCR (RT-PCR) with mRNA, to amplify nucleic acid molecules encoding open reading frames. Such nucleic acid molecules can be sequenced to identify those that encode an isoform. In one example, nucleic acid molecules of different sizes (e.g. molecular masses) from a predicted size (such as a size predicted for an encoded wildtype or predominant form) are chosen as candidate isoforms. Such nucleic acid molecules then can be analyzed, such as by a method described herein, to further select isoform-encoding molecules having specified properties.

Computational analysis is performed using the obtained nucleic acid sequences to further select candidate isoforms. For example, cDNA sequences are aligned with a genomic sequence of a selected candidate gene. Such alignments can be performed manually or by using bioinformatics programs such as SIM4, a computer program for analysis of splice variants. Sequences with canonical donor-acceptor splicing sites (e.g. GT-AG) are selected. Molecules can be chosen which represent alternatively spliced products such as exon deletion, exon retention, exon extension and intron retention.

Sequence analysis of isolated nucleic acid molecules also can be used to further select isoforms that retain or lack a domain and/or a function compared to a wildtype or predominant form. For example, isoforms encoded by isolated nucleic acid molecules can be analyzed using bioinformatics programs such as described herein to identify protein domains. Isoforms then can be selected which retain or lack a domain or a portion thereof.

In one embodiment, isoforms are selected that lack a SerP domain or portion thereof sufficient to reduce an activity. For example, isoforms are selected that lack one or more amino acids of the SerP domain or have a disruption of the SerP domain, such as an insertion of one or more amino acids. Isoforms also can be selected that lack a SerP domain or portion thereof and have one or more amino acids operatively linked in place of the missing domain or portion of a domain. Such isoforms can be the result of alternative splicing events such as exon extension, intron retention, exon deletion and exon insertion. In some case, such alternatively spliced RNA molecules alter the reading frame of an RNA and/or operatively link sequences not found in an RNA encoding a wildtype or predominant form.

In another embodiment, isoforms are selected that lack at least one kringle domain or part of a kringle domain. For example, an isoform is selected that lacks any one or more and/or part of any one or more of the K1, K2, K3, or K4 domains. Such isoforms can include those that lack one or more amino acids of the K1 domain. For example, HGF isoforms can lack one or more of amino acids corresponding to amino acids 128-206 of SEQ ID NO:3. Such isoforms also can lack a SerP domain. The isoforms can be the result of alternative splicing events such as exon extension, intron retention, exon deletion and exon insertion. In some case, such alternatively spliced RNA molecules alter the reading frame of an RNA and/or operatively link sequences not found in an RNA encoding a wildtype or predominant form. Such isoforms can include additional amino acid sequences not found in a wildtype or predominant form of HGF. In one example, an additional amino acid sequence is contained at the C-terminus of an HGF isoform.

Nucleic acid molecules can be selected which encode an HGF isoform and have an activity that differs from a wildtype or predominant form of HGF. In one example, HGF isoforms are selected that lack a SerP domain such that the isoforms do not stimulate signal transduction by MET. In another example, HGF isoforms are selected that lack all or part of at least one kringle domain, but maintain binding to MET and/or another cell surface interacting partner, such as for example heparin, and that alter one or more biological activities of a growth factor receptor stimulated by a its growth factor ligand, including ligand interactions and signal transduction.

2. Identification of Allelic and Species Variants of Isoforms

Allelic variants and species variants of ligand isoforms, such as HGF isoforms, can be generated or identified. Such variants differ in one or more amino acids from a particular HGF isoform or cognate HGF. Allelic variation occurs among members of a population and species variation occurs between species. For example, isoforms can be derived from different alleles of a gene; each allele can have one or more amino acid differences from the other. Such alleles can have conservative and/or non-conservative amino acid differences. Allelic variants also include isoforms produced or identified from different subjects, such as individual subjects or animal models or other animals. Amino acid changes can result in modulation of an isoform's biological activity. In some cases, an amino acid difference can be “silent”, having no or virtually no detectable affect on a biological activity. Allelic variants of isoforms also can be generated by mutagenesis. Such mutagenesis can be random or directed. For example, allelic variant isoforms can be generated that alter amino acid sequences or a potential glycosylation site to effect a change in glycosylation of an isoform, including alternate glycosylation, increased or inhibition of glycosylation at a site in an isoform. Allelic variant isoforms can be are at least 90% identical in sequence to an isoform. Generally, an allelic variant isoform from the same species is at least 95%, 96%, 97%, 98%, 99% identical to an isoform, typically an allelic variant is 98%, 99%, 99.5% identical to an isoform.

For example, HGF isoforms, including HGF isoforms herein, can include allelic variation in the HGF polypeptide. For example, an HGF isoform can include one or more amino acid differences present in an allelic variant of a cognate HGF. In one example, an HGF isoform includes one or more allelic variations as set forth in SEQ ID NO:16. Examples of allelic variation include variants in the N-terminal domain, kringle domains, or SerP domain, including, but not limited to, amino acid variation at positions corresponding to amino acids 78, 82, 153, 180, 293, 300, 304, 317, 325, 330, 336, 387, 416, 494, 505, or 509 set forth in SEQ ID NO:16. HGF isoforms also include species variants of a cognate HGF.

### E. Exemplary HGF Isoforms

1. HGF Isoforms

Isoforms of HGF are provided. In particular, isoforms of HGF that are truncated but that include at least all or part of the K4 region but lack one or more of all of part of the N-terminal domain, K1, K2, K3, or SerP domain are provided.

2. HGF Intron Fusion Proteins

Provided herein are exemplary HGF isoforms that have an altered domain organization compared to a cognate HGF due to the retention of an intron-encoded sequence in the nucleic acid molecule that encodes the HGF isoform.

HGF isoforms provided herein are encoded by nucleic acid molecules that include all or a portion of any intron of an HGF, except for intron 5, operatively linked to an exon. The intron portion can include one codon, including a stop codon, which results in an HGF isoform that ends at the end of the exon, or can include more codons so that the HGF isoform includes intron encoded residues.

The intron/exon structure of an exemplary HGF isoform is depicted in FIG. 1. A sequence therefor is set forth in SEQ ID NO:1. In the exemplary genomic sequence of HGF set forth in SEQ ID NO:1, HGF isoforms provided herein can include all or a portion of any intron of an HGF, such as all of part of intron 1 containing nucleotides 254-7264, intron 2 containing nucleotides 7432-11333, intron 3 containing nucleotides 11446-12833, intron 4 containing nucleotides 12949-17874, intron 6 containing nucleotides 25138-26665, intron 7 containing nucleotides 26785-40357, intron 8 containing nucleotides 40533-44119, intron 9 containing nucleotides 44248-49289, intron 10 containing nucleotides 49393-52771, intron 11 containing nucleotides 52906-58617, intron 12 containing nucleotides 58657-59893, intron 13 containing nucleotides 59992-62772, intron 14 containing nucleotides 62848-63709, intron 15 containing nucleotides 63851-64383, intron 16 containing nucleotides 64491-64601, and intron 17 containing nucleotides 64748-67379. Exemplary HGF isoforms retain all or part of intron 11 or intron 13 of an HGF gene. An intron-encoded portion of an isoform can exist N-terminally, C-terminally, or internally to an exon sequence(s) operatively linked to the intron.

In one embodiment, intron fusion proteins of HGF, or allelic variants thereof, provided herein lack all or part of a domain of the full length cognate HGF such that the HGF isoform exhibits an antagonistic and/or anti-angiogenic activity. Isoforms provided herein lack one or more of part of an N-terminal domain, part of a K1 domain, part of a K2 domain, part of a K3 domain, part of a K4 domain, and all or part of a SerP domain of a cognate HGF, or combinations thereof. The truncations and deletions when selected produce an isoform with the aforementioned activity.

An isoform includes intron-encoded amino acids from any one or more of introns 1, 2, 3, 4, 6, 7, 8, 9, 10, 11, 12, 13, 14, 15, 16, or 17 internally within the isoform, or at the N- or C-terminus or the isoform is truncated at the end of an exon. HGF isoforms and allelic variants thereof provided herein can exhibit anti-angiogenic activity. For example, an isoform can lack all or part of an N-terminal domain, part of a K1 domain, all or part of a K2 domain, all or part of a K3 domain, all or part of a K4 domain, or all or part of a SerP domain, or combinations thereof. An isoform can include intron-encoded amino acids from any one or more of introns 1, 2, 3, 4, 6, 7, 8, 9, 10, 11, 12, 13, 14, 15, 16, or 17 internally within the isoform, or at the N- or C-terminus. In some examples, an isoform that is anti-angiogenic also can exhibit antagonistic activity.

Among the HGF isoforms provided herein is an isoform whose encoding nucleic acid molecule is designated SR023A02. Nucleic acid and amino acid sequences therefor are set forth in SEQ ID NOS:9 or 17. Clone SR023A02 contains 1471 bases, including an intron portion at the C-terminus-encoding end. The intron portions contains the first 34 nucleotides of intron 11. The intron 11 portion encodes three amino acids followed by a stop codon. In the clone this portion is operatively linked to an open reading frame of exons 1-11. The encoded HGF isoform is truncated compared to the cognate HGF and includes the three intron encoded amino acids at the C-terminus. SR023A02 encodes a 467 amino acid HGF isoform polypeptide whose sequence is set forth in SEQ ID NO:10 or 18, which each encode the SR023A02 isoform but differ in two amino acids. SEQ ID NO:10 contains a Leu at position 82 and a Ser at position 320. SEQ ID NO:18 has a Phe and Pro at these positions, respectively, which correspond to the amino acids of the cognate HGF set forth in SEQ ID NO:3. The SR023A02 isoform contains a signal sequence at the N-terminus at amino acids 1-31 and an N-terminal domain following the signal sequence at amino acids 34-124. Compared with a cognate receptor set forth in SEQ ID NO:3, the SR023A02 encoded HGF isoform contains a deletion of amino acids 161-165 in the K1 domain (see. e.g., FIG. 2). Further, this isoform includes a K2, K3, and K4 domain corresponding to amino acids 211-288, 305-383, and 391-469, respectively, of SEQ ID NO:3 and it lacks the SerP domain. The isoform encoded by SR023A02 also includes an additional 3 amino acids following the K4 domain (amino acids 465-467) not present in the cognate HGF set forth as SEQ ID NO:3. Also provided are allelic and species variants of SR023A02. These are produced by isolating them from another source or synthesizing them based on the known sequences of the cognate receptor. These differ at the residue in which the encoding nucleic acid differs from the SEQ ID NO:2. Exemplary HGF allelic variants are set forth in SEQ ID NO:15 or 16.

Provided herein is another exemplary HGF isoform that is encoded by a nucleic acid molecule designated SR023A08, whose sequence is set forth in SEQ ID NOS:11 or 19. SR023A08 contains 1495 bases, including an intron portion at the C-terminus containing the first 34 nucleotides of intron 11. The intron 11 portion encodes three amino acids followed by a stop codon that is operatively linked with an open reading frame of exons 1-11 of the encoded polypeptide thereby resulting in an HGF isoform that is truncated compared to a cognate HGF. The HGF isoform encoded by SR023A08 contains 472 amino acids set forth in SEQ ID NO:12 or 20, which each encode the SR023A08 isoform but differ in one amino acid. SEQ ID NO:12 contains a Lys at position 304 while SEQ ID NO:20 has Glu at this position which corresponds to the amino acid of the cognate HGF set forth in SEQ ID NO:3. This isoform includes a signal sequence at the N-terminus at amino acids 1-31, an N-terminal domain at amino acids 34-124, a K1 domain at amino acids 128-206, a K2 domain at amino acids 211-288, a K3 domain at amino acids 305-383, and a K4 domain at amino acids 391-469 (see e.g., FIG. 2). The HGF isoform encoded by SR023A08 lacks a SerP domain, but contains an additional 3 amino acids following the K4 domain (amino acids 470-472) not present in the cognate HGF set forth as SEQ ID NO:3. SR023A08 variants, including allelic and species variants are provided. These differ at the residue in which the encoding nucleic acid differs from the SEQ ID NO:2. Exemplary HGF allelic variants are set forth in SEQ ID NO:16 and encoded in SEQ ID NO: 15.

Another exemplary HGF isoform encoded by the clone SR023E09 is provided. The encoding nucleic acid sequence set forth in SEQ ID NO:13. This clone contains 1613 bases, including an intron portion at the C-terminus containing the first 66 nucleotides of intron 13. The intron 13 portion encodes a stop codon that is operatively linked with an open reading frame of exons 1-13 of the encoded polypeptide thereby resulting in an HGF isoform that is truncated compared to a cognate HGF. The SR023E09 encoded isoform is 514 amino acids in length, including the signal sequence. The amino acid sequence of the isoform is set forth in SEQ ID NO:14. The isoform contains an N-terminal signal sequence at amino acids 1-31, an N-terminal domain at amino acids 34-124, a K1 domain at amino acids 128-206, a K2 domain at amino acids 211-288, a K3 domain at amino acids 305-383, and a K4 domain at amino acids 391-469 (see e.g., FIG. 2). This isoform is truncated after amino acid 514 and thereby lacks part of the SerP domain corresponding to amino acids 515-728 of a cognate HGF set forth in SEQ ID NO:3. Variants, including allelic and species variants of the SR023A08 encoded HGF isoform are provided. These include allelic variations, such as any one of the allelic variations set forth in SEQ ID NO:15 or 16 of a cognate HGF nucleic acid or polypeptide, respectively.

### F. Methods for Producing Nucleic Acids Encoding HGF Isoform Polypeptides

Exemplary methods for generating HGF isoform nucleic acid molecules and polypeptides include molecular biology techniques known to one of skill in the art. Such methods include in vitro synthesis methods for nucleic acid molecules such as PCR, synthetic gene construction and in vitro ligation of isolated and/or synthesized nucleic acid fragments. HGF isoform nucleic acid molecules also can be isolated by cloning methods, including PCR of RNA and DNA isolated from cells and screening of nucleic acid molecule libraries by hybridization and/or expression screening methods.

HGF isoform polypeptides can be generated from HGF isoform nucleic acid molecules using in vitro and in vivo synthesis methods. HGF isoforms can be expressed in any organism suitable to produce the required amounts and forms of the isoform needed for administration and treatment. Expression hosts include prokaryotic and eukaryotic organisms such as E. coli, yeast, plants, insect cells, mammalian cells, including human cell lines and transgenic animals. HGF isoforms also can be isolated from cells and organisms in which they are expressed, including cells and organisms in which isoforms are produced recombinantly and those in which isoforms are synthesized without recombinant means such as genomically-encoded isoforms produced by alternative splicing events.

1. Synthetic Genes and Polypeptides

HGF isoform nucleic acid molecules and polypeptides can be synthesized by methods known to one of skill in the art using synthetic gene synthesis. In such methods, a polypeptide sequence of an HGF isoform is “back-translated” to generate one or more nucleic acid molecules encoding an isoform. The back-translated nucleic acid molecule is then synthesized as one or more DNA fragments such as by using automated DNA synthesis technology. The fragments are then operatively linked to form a nucleic acid molecule encoding an isoform. Nucleic acid molecules also can be joined with additional nucleic acid molecules such as vectors, regulatory sequences for regulating transcription and translation and other polypeptide-encoding nucleic acid molecules. Isoform-encoding nucleic acid molecules also can be joined with labels such as for tracking, including radiolabels, and fluorescent moieties.

The process of back-translation uses the genetic code to obtain a nucleotide gene sequence for any polypeptide of interest, such as an HGF isoform. The genetic code is degenerate, 64 codons specify 20 amino acids and 3 stop codons. Such degeneracy permits flexibility in nucleic acid design and generation, allowing, for example, restriction sites to be added to facilitate the linking of nucleic acid fragments and the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences. Back-translation is performed by selecting codons that encode a polypeptide. Such processes can be performed manually using a table of the genetic code and a polypeptide sequence. Alternatively, computer programs, including publicly available software can be used to generate back-translated nucleic acid sequences.

To synthesize a back-translated nucleic acid molecule, any method available in the art for nucleic acid synthesis can be used. For example, individual oligonucleotides corresponding to fragments of an HGF isoform-encoding sequence of nucleotides are synthesized by standard automated methods and mixed together in an annealing or hybridization reaction. Such oligonucleotides are synthesized such that annealing results in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to an HGF isoform-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an isoform-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to isoform-encoding nucleic acid molecules. Such regions include, but are not limited to, sequences to facilitate uptake of an isoform into specific target cells, or otherwise enhance the pharmacokinetics of the synthetic gene.

HGF isoforms also can be synthesized using automated synthetic polypeptide synthesis. Cloned and/or in silico-generated polypeptide sequences can be synthesized in fragments and then chemically linked. Alternatively, isoforms can be synthesized as a single polypeptide. Such polypeptides then can be used in the assays and treatment administrations described herein.

2. Methods of Cloning and Isolating HGF Isoforms

HGF isoforms can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding an isoform, including for example, polymerase chain reaction (PCR) methods. A nucleic acid containing material can be used as a starting material from which an isoform-encoding nucleic acid molecule can be isolated. For example, DNA and mRNA preparations, cell extracts, tissue extracts, fluid samples (e.g. blood, serum, saliva), and samples from healthy and/or diseased subjects can be used in amplification methods. Nucleic acid libraries also can be used as a source of starting material. Primers can be designed to amplify an isoform. For example, primers can be designed based on expressed sequences from which an isoform is generated. Primers can be designed based on back-translation of an isoform amino acid sequence. Nucleic acid molecules generated by amplification can be sequenced and confirmed to encode an isoform.

Nucleic acid molecules encoding isoforms also can be isolated using library screening. For example, a nucleic acid library representing expressed RNA transcripts such as cDNA molecules can be screened by hybridization with nucleic acid molecules encoding HGF isoforms or portions thereof. For example, an intron sequence or portion thereof from an HGF gene can be used to screen for intron retention containing molecules based on hybridization to homologous sequences. Expression library screening can be used to isolate nucleic acid molecules encoding an HGF isoform. For example, an expression library can be screened with antibodies that recognize a specific isoform or a portion of an isoform. Antibodies can be obtained and/or prepared which specifically bind an HGF isoform or a region or peptide contained in an isoform. Antibodies which specifically bind an isoform can be used to screen an expression library containing nucleic acid molecules encoding an isoform. Exemplary methods for producing isoform-specific antibodies are described below.

3. Expression Systems

HGF isoforms, including natural and combinatorial intron fusion proteins, can be produced by any method known to those of skill in the art including in vivo and in vitro methods. HGF isoforms can be expressed in any organism suitable to produce the required amounts and forms of HGF isoforms needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of HGF isoforms. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

HGF isoforms also can be utilized or expressed as protein fusions. For example, an isoform fusion can be generated to add additional functionality to an isoform. Examples of isoform fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Expression

Prokaryotes, especially E. coli, provide a system for producing large amounts of proteins such as HGF isoforms. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Isoforms can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach is the expression of HGF isoforms in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are well known yeast expression hosts that can be used for production of HGF isoforms. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as, for example, the Kex-2 protease, can be engineered to remove the fused sequences from the expressed polypeptides as they exit the secretion pathway. Yeast also are capable of glycosylation at Asn-X-Ser/Thr motifs.

C. Insect Cells

Insect cells, particularly using baculovirus expression, are useful for expressing polypeptides such as HGF isoforms. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express HGF isoforms. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha-1-antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express HGF isoforms. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce CSR isoforms (see for example, Mayfield et al. (2003) PNAS 100:438-442). Because plants have different glycosylation patterns than mammalian cells, this can influence the choice of CSR isoforms produced in these hosts.

### G. Isoform Conjugates

A variety of synthetic conjugates of HGF isoforms are provided. In one example, HGF isoforms are provided as fusion proteins whereby an HGF isoform is linked directly or indirectly to another polypeptide, such as a polypeptide that promotes secretion of an isoform or to a multimerization domain. In some examples, a fusion protein can result in a chimeric polypeptide. For example, a chimera can include a polypeptide in which the extracellular domain portion and C-terminal portion, such as an intron encoded portion, are from different isoforms. Also included among synthetic forms are conjugates in which the isoform or intron-encoded portion thereof is linked directly or via a linker to another agent, such as a targeting agent or target agent or to any other molecule that presents an HGF isoform or intron-encoded portion of an HGF isoform to a cell surface receptor (CSR), such as MET, so that an activity of the CSR is modulated. Also provided are “peptidomimetic” isoforms in which one or more bonds in the peptide backbone is (are) replaced by a bioisostere or other bond such that the resulting polypeptide peptidomimetic has improved properties, such as resistance to proteases, compared to the unmodified form.

HGF isoform conjugates can be designed and produced with one or more modified properties. These properties include, but are not limited to, increased production including increased secretion or expression. For example, an HGF isoform can be modified to exhibit improved secretion compared to an unmodified HGF isoform. Other properties include increased protein stability, such as an increased protein half-life, increased thermal tolerance and/or resistance to one or more proteases, and increased ability to dimerize or form multimers. For example, an HGF isoform can be modified to increase protein stability in vitro and/or in vivo. In vivo stability can include protein stability under particular administration conditions such as stability in blood, saliva, and/or digestive fluids.

HGF isoforms also can be modified to exhibit modified properties without producing a conjugated polypeptide using any methods known in the art for modification of proteins. Such methods can include site-directed and random mutagenesis. Non-natural amino acids and/or non-natural covalent bonds between amino acids of the polypeptide can be introduced into an HGF isoform to increase protein stability. In such modified HGF isoforms, the biological function of the isoform can remain unchanged compared to the unmodified isoform. In some examples, a modified HGF isoform also can be provided as a conjugate such as a fusion protein, chimeric protein, or other conjugate provided herein. Assays such as the assays for biological function provided herein and known in the art can be used to assess the biological function of a modified HGF isoform.

Linkage of a synthetic HGF isoform as a fusion protein or synthetic conjugate can be direct or indirect. In some examples, linkage can be facilitated by nucleic acid linkers such as restriction enzyme linkers, or other peptide linkers that promote the folding or stability of an encoded polypeptide. Linkage of a polypeptide conjugate also can be by chemical linkage or facilitated by heterobifunctional linkers, such as any known in the art or provided herein. Exemplary peptide linkers and heterobifunctional cross-linking reagents are provided below. For example, exemplary peptide linkers include, but are not limited to, (Gly4Ser)n, (Ser4Gly)n and (AlaAlaProAla)n (see e.g., SEQ ID NO. 270) in which n is 1 to 4, such as 1, 2, 3 or 4, such as:

| | |
|---|---|
| (1) | Gly4Ser with NcoI ends SEQ ID NO. 266 |
| | CCATGGGCGG CGGCGGCTCT GCCATGG |
| (2) | (Gly4Ser)2 with NcoI ends SEQ ID NO. 267 |
| | CCATGGGCGG CGGCGGCTCT GGCGGCGGCG GCTCTGCCAT GG |
| (3) | (Ser4Gly)4 with NcoI ends SEQ ID NO. 268 |
| | CCATGGCCTC GTCGTCGTCG GGCTCGTCGT CGTCGGGCTC |
| | GTCGTCGTCG GGCTCGTCGT CGTCGGGCGC CATGG |
| (4) | (Ser4Gly)2 with NcoI ends SEQ ID NO. 269 |
| | CCATGGCCTC GTCGTCGTCG GGCTCGTCGT CGTCGGGCGC |
| | CATGG |
| (5) | (AlaAlaProAla)n, where n is 1 to 4, such as 2 |
| | or 3 (see e.g., SEQ ID NO:270) |

Numerous heterobifunctional cross-linking reagents that are used to form covalent bonds between amino groups and thiol groups and to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad Sci. 84:308-312; Walden et al. (1986) J. Mol. Cell Immunol. 2:191-197; Carlsson et al. (1978) Biochem. J. 173: 723-737; Mahan et al. (1987) Anal. Biochem. 162:163-170; Wawrzynczak et al. (1992) Br. J. Cancer 66:361-366; Fattom et al. (1992) Infection & Immun. 60:584-589). These reagents may be used to form covalent bonds between the N-terminal portion and C-terminus intron-encoded portion or between each of those portions and a linker. These reagents include, but are not limited to: N-succinimidyl-3-(2-pyridyldithio)propionate (SPDP; disulfide linker); sulfosuccinimidyl 6-[3-(2-pyridyldithio)propion [symbol] amido]hexanoate (sulfo-LC-SPDP); succinimidyloxycarbonyl-α-methyl benzyl thiosulfate (SMBT, hindered disulfate linker); succinimidyl 6-[3-(2-pyridyldithio)propionami [symbol] do] [symbol] hexanoate (LC-SPDP); sulfosuccinimidyl 4-(N-maleimidomethyl)cyclohexane-1-carboxylate (sulfo-SMCC); succinimi [symbol] dyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfo-succinimidyl 7-azido-4-methylcoumarin-3-acetate (SAMCA); sulfosuccinimidyl-6-[alpha-methyl-alpha-(2-pyridyldithio)toluamido]-hexanoate (sulfo-LC-SMPT); 1,4-di-[3′-(2′-pyridyldithio)propion-amido]butane (DPDPB); 4-succinimidyloxycarbonyl-α-methyl-α-(2-pyridylthio)toluene (SMPT, hindered disulfate linker); sulfosuccinimidyl-6-[α-methyl-α-(2-pyrimiyldi-thio)toluamido]hexanoate (sulfo-LC-SMPT); m-maleimidobenzoyl-N-hydroxy-succinimide ester (MBS); m-maleimidobenzoyl-N-hydroxysulfo-succinimide ester (sulfo-MBS); N-succinimidyl(4-iodoacetyl)aminobenzoate (SIAB; thioether linker); sulfosuccinimidyl-(4-iodoacetyl)amino benzoate (sulfo-SIAB); succinimidyl-4-(p-maleimi-dophenyl)butyrate (SMPB); sulfosuccinimidyl-4-(p-maleimido-phenyl)butyrate (sulfo-SMPB); azidobenzoyl hydrazide (ABH). These linkers, for example, can be used in combination with peptide linkers, such as those that increase flexibility or solubility or that provide for or eliminate steric hindrance. Any other linkers known to those of skill in the art for linking a polypeptide molecule to another molecule can be employed. General properties are such that the resulting molecule is biocompatible (for administration to animals, including humans) and such that the resulting molecule modulates the activity of a cell surface molecule, such as a MET receptor, angiogenic molecule, or other cell surface molecule or receptor.

Pharmaceutical compositions can be prepared that contain HGF isoform conjugates and treatment effected by administering a therapeutically effective amount of a conjugate, for example, in a physiologically acceptable excipient. HGF isoform conjugates also can be used in in vivo therapy methods such as by delivering a vector containing a nucleic acid encoding an HGF isoform conjugate as a fusion protein.

1. Isoform Fusions

HGF isoform fusions include operative linkage of a nucleic acid sequence encoding HGF with another nucleic acid molecule. Nucleic acid molecules that can be joined to an HGF isoform, include but are not limited to, promoter sequences designed to facilitate intracellular protein expression, secretion sequences designed to facilitate protein secretion, regulatory sequences for regulating transcription and translation, molecules that regulate the serum stability of an encoded polypeptide such as portions of CD45 or an Fc portion of an immunoglobulin, and other polypeptide-encoding nucleic acid molecules such as those encoding a targeted agent or targeting agent, or those encoding all or part of another ligand or cell surface receptor intron fusion protein. The fusion sequence can be a component of an expression vector, or it can be part of an isoform nucleic acid sequence that is inserted into an expression vector. The fusion can result in a chimeric protein encoded by two or more genes, or the fusion can result in a protein sequence encoding only an HGF isoform polypeptide, such as if the fused sequence is a signal sequence that is cleaved off following secretion of the polypeptide into the secretory pathway. In one example, a nucleic acid fused to all or part of an HGF isoform can include any nucleic acid sequence that improves the production of an isoform such as a promoter sequence, epitope or fusion tag, or a secretion signal. In another example, an HGF isoform fusion can include fusion with a targeted agent or targeting agent to produce an HGF isoform conjugate such as described below. Additionally, a nucleic acid encoding all or part of an HGF isoform can be joined to a nucleic acid encoding another ligand or cell surface receptor intron fusion isoform, or intron portion thereof, thereby generating a chimeric intron fusion protein. Exemplary HGF chimeras are described below. HGF isoform-multimerization domain, such Fc domains, fusions are provided.

Encoded HGF isoform fusion proteins can contain additional amino acids which do not adversely affect the activity of a purified isoform protein. For example, additional amino acids can be included in the fusion protein as a linker sequence which separate the encoded isoform protein from the encoded fusion sequence in order to provide, for example, a favored steric configuration in the fusion protein. The number of such additional amino acids which may serve as separators may vary, and generally do not exceed 60 amino acids. Exemplary linker sequences are provided below. In another example, a fusion protein can contain amino acid residues encoded by a restriction enzyme linker sequence. In an additional example, an isoform fusion protein can contain selective cleavage sites at the junction or junctions between the fusion of an HGF isoform with another molecule. For example, such selective cleavage sites may comprise one or more amino acid residues which provide a site susceptible to selective enzymatic, proteolytic, chemical, or other cleavage. In one example, the additional amino acids can be a recognition site for cleavage by a site-specific protease. The fusion protein can be further processed to cleave the fused polypeptide therefrom; for example, if the isoform protein is fused to an epitope tag but is required without additional amino acids such as for therapeutic purposes.

a. HGF Isoform Fusions for Improved Production of HGF Isoform Polypeptides

Provided herein are nucleic acid sequences encoding HGF fusion polypeptides for the improved production of an HGF isoform. A nucleic acid of an HGF isoform, such as set forth in any one of SEQ ID NOS: 9, 11, 13, 17, or 19 can be fused to a homologous or heterologous precursor sequence that substitutes for and/or provides for a functional secretory sequence. Other exemplary HGF isoforms can include other natural and engineered isoform variants of a cognate HGF such as set forth in any one of SEQ ID NOS: 21, 23, 25, 27, 29, and 31 and encoding a polypeptide set forth in any one of SEQ ID NOS: 22, 24, 26, 28, 30, or 32. In one example, an isoform, such as an intron fusion protein isoform, containing a native endogenous precursor signal sequence of a cognate HGF ligand can have its precursor sequence replaced with a heterologous or homologous precursor sequence, such as a precursor sequence of tissue plasminogen activator or any other signal sequence known to one of skill in the art, to improve the secretion and production of an HGF isoform polypeptide. The precursor sequence is most effectively utilized by locating it at the N-terminus of a recombinant protein to be secreted from the host cell. A nucleic acid precursor sequence can be operatively joined to a nucleic acid containing the coding region of an HGF isoform in such a manner that the precursor sequence coding region is upstream of (that is, 5′ of), and in the same reading frame as, the isoform coding region to provide an isoform fusion. The isoform fusion can be expressed in a host cell to provide a fusion polypeptide comprising the precursor sequence joined, at its carboxy terminus, to an HGF isoform at its amino terminus. The fusion polypeptide can be secreted from a host cell. Typically, a precursor sequence is cleaved from the fusion polypeptide during the secretion process, resulting in the accumulation of a secreted isoform in the external cellular environment or, in some cases, in the periplasmic space.

Optionally an HGF isoform, including an intron fusion protein that is a fusion nucleic acid also can include operative linkage with another nucleic acid sequence or sequences, such as a sequence that encodes a fusion tag, that promotes the purification and/or detection of an isoform polypeptide. Non-limiting examples of fusion tags include a myc tag, Poly-His tag, GST tag, Flag tag, fluorescent or luminescent moiety such as GFP or luciferase, or any other epitope or fusion tag known to one of skill in the art. In other embodiments, a nucleic acid sequence of an HGF isoform can contain an endogenous signal sequence and can include fusion with a nucleic acid sequence encoding a fusion tag or tags. Many precursor sequences, including signal sequences and prosequences, and/or fusion tag sequences have been identified and are known in the art, such as but not limited to, those provided and described herein, and are contemplated to be used in conjunction with an isoform nucleic acid molecule. A precursor sequence may be homologous or heterologous to an isoform gene or cDNA, or a precursor sequence can be chemically synthesized. In most cases, the secretion of an isoform polypeptide from a host cell via the presence of a signal peptide and/or propeptide will result in the removal of the signal peptide or propeptide from the secreted intron fusion protein polypeptide.

i. Tissue Plasminogen Activator

Tissue plasminogen activator (tPA) is a serine protease that regulates hemostasis by converting the zymogen plasminogen to its active form, plasmin. Like other serine proteases, tPA is synthesized and secreted as an inactive zymogen that is activated by proteolytic processing. Specifically, the mature partially active single chain zymogen form of tPA can be further processed into a two-chain fully active form by cleavage after Arg-310 of SEQ ID NO:255 catalyzed by plasmin, tissue kallikrein or factor Xa. tPA is secreted into the blood by endothelial cells in areas immediately surrounding blood clots, which are areas rich in fibrin. tPA regulates fibrinolysis due to its high catalytic activity for the conversion of plasminogen to plasmin, a regulator of fibrin clots. Plasmin also is a serine protease that becomes converted into a catalytically active, two-chain form upon cleavage of its zymogen form by tPA. Plasmin functions to degrade the fibrin network of blood clots by cutting the fibrin mesh at various places, leading to the production of circulating fragments that are cleared by other proteinases or by the kidney and liver.

The precursor polypeptide of tPA includes a pre-sequence and pro-sequence encoded by residues 1-35 of a full-length tPA sequence set forth in SEQ ID NO:255 and exemplified in SEQ ID NO:253. The precursor sequence of tPA contains a signal sequence including amino acids 1-23 and also contains two pro-sequences including amino acids 24-32 and 33-35 of an exemplary tPA sequence set forth in SEQ ID NO: 253 or 255. The signal sequence of tPA is cleaved co-translationally in the ER and a pro-sequence is removed in the Golgi apparatus by cleavage at a furin processing site following the sequence RFRR occurring at amino acids 29-32 of the exemplary sequences set forth in SEQ ID NO:253 or 255. Furin cleavage of a tPA pro-sequence retains a three amino acid pro-sequence and exopeptidase cleavage site GAR, set forth as amino acids 33-35 of an exemplary tPA sequence set forth in SEQ ID NO: 253 or 255, within a mature polypeptide tPA sequence. The cleavage of the retained pro-sequence site is mediated by a plasmin-like extracellular protease to obtain a mature tPA polypeptide beginning at Ser36 set forth in SEQ ID NO:253 or 255. Inclusion of a protease inhibitor, such as for example aprotinin, in the culture medium can prevent exopeptidases cleavage and thereby retain a GAR pro-sequence in the mature polypeptide of tPA (Berg et al., (1991) Biochem Biophys Res Comm, 179:1289).

Typically, tPA is secreted by the constitutive secretory pathway, although in some cells tPA is secreted in a regulated manner. For example, in endothelial cells regulated secretion of tPA is induced following endothelial cell activation, for example, by histamine, platelet-activating factor or purine nucleotides, and requires intraendothelial Ca2+ and cAMP signaling (Knop et al., (2002) Biochem Biophys Acta 1600:162). In other cells, such as for example neural cells, specific stimuli that can induce secretion of tPA include exercise, mental stress, electroconvulsive therapy, and surgery (Parmer et al., (1997) J Biol Chem 272:1976). The mechanism mediating the regulated secretion of tPA requires signals on the tPA polypeptide itself, whereas the signal sequence of tPA efficiently mediates constitutive secretion of tPA since a GFP molecule operatively linked only to the signal sequence of tPA is constitutively secreted in the absence of carbachol stimulation (Lochner et al., (1998) Mol Biol Cell, 9:2463). In the absence of a tPA signal sequence, a tPA/GFP hybrid protein is not secreted from cells.

An exemplary tPA precursor sequence including a pre/propeptide sequence of tPA is set forth in SEQ ID NO: 253, and is encoded by a nucleic acid sequence set forth in SEQ ID NO:252. The signal sequence of tPA includes amino acids 1-23 of SEQ ID NO:255 and the pro-sequence includes amino acids 24-35 of SEQ ID NO:255 whereby a furin-cleaved pro-sequence includes amino acids 24-32 and a plasmin-like exoprotease-cleaved pro-sequence includes amino acids 33-35. Allelic variants of a tPA pre/prosequence are also provided herein, such as those set forth in SEQ ID NOS:256 or 257. Further, isoform protein fusions of a pre/prosequence of tPA of mammalian and non-mammalian origin are contemplated and exemplary sequences are set forth in SEQ ID NOS:258-265.

ii. tPA-HGF Isoform Fusions

Provided herein are nucleic acid sequences encoding tPA-HGF isoform polypeptides, for the improved production of an HGF intron fusion protein isoform. Nucleic acid sequences encoding HGF isoforms, including intron fusion protein isoforms of HGF, or allelic variants thereof, such as any one of SEQ ID NOS: 9, 11, or 13, encoding amino acids set forth in SEQ ID NOS:10, 12, 14, 18, or 20 operatively linked to a tPA pre/prosequence are provided. A tPA pre/prosequence can include a tPA pre/prosequence set forth as SEQ ID NO:252 encoding amino acids set forth as 1-35 in SEQ ID NO:253. In some examples, a tPA pre/pro sequence can replace the endogenous precursor signal sequence of HGF and/or provide for an optimal precursor sequence for the secretion of an intron fusion protein polypeptide.

In other embodiments, an HGF isoform or allelic variants thereof, set forth in any one of SEQ ID NOS: 9, 11, or 13, encoding amino acids set forth in SEQ ID NOS:10, 12, 14, 18, or 20, can be operatively linked to part of a tPA pre/prosequence including the nucleic acid sequence up to the furin cleavage site of a pre/prosequence of tPA (encoded amino acids 1-32 of an exemplary tPA pre-prosequence set forth in SEQ ID NO:253), thereby excluding nucleic acids encoding amino acids GAR (encoded amino acids 33-35 of an exemplary tPA pre-prosequence set forth in SEQ ID NO:253). Additionally, a nucleic acid sequence of an HGF isoform or allelic variants thereof, such as set forth in any one of SEQ ID NOS: 9, 11, or 13, encoding amino acids set forth in SEQ ID NOS:10, 12, 14, 18, or 20, can include operative linkage with allelic variants of all or part of a tPA pre/prosequence, such as set forth in SEQ ID NOS: 252 or 253 or can include operative linkage with all or part of other tPA pre/prosequences of mammalian and non-mammalian origin, such as set forth in any one of SEQ ID NOS:258-265. HGF intron fusion protein-tPA pre/pro fusion sequences provided herein can exhibit enhanced cellular expression and secretion of an HGF isoform polypeptide for improved production.

In another embodiment, a nucleic acid sequence encoding an HGF isoform or allelic variant thereof, such as any one of SEQ ID NOS: 9, 11, or 13, encoding amino acids set forth in SEQ ID NOS:10, 12, 14, 18, or 20, can include operative linkage with a presequence (signal sequence) only of a tPA pre/prosequence such as an exemplary signal sequence encoding amino acids 1-23 of an exemplary tPA pre/prosequence set forth as SEQ ID NO:253. HGF intron fusion protein-tPA presequence fusions provided herein can exhibit enhanced cellular expression and secretion of an HGF isoform polypeptide for improved production.

In an additional embodiment, a nucleic acid sequence encoding an HGF isoform or allelic variant thereof, such as any one of SEQ ID NOS: 9, 11, or 13, encoding amino acids set forth in SEQ ID NOS:10, 12, 14, 18, or 20, that contains an endogenous signal sequence of a cognate HGF ligand can include a fusion with a tPA prosequence where insertion of a tPA prosequence is between the HGF isoform endogenous signal sequence and the HGF isoform coding sequence. In one example, a tPA prosequence includes a nucleic acid sequence encoding amino acids 24-32 of an exemplary tPA pre/prosequence set forth as SEQ ID NO:253. In another example, a tPA pro-sequence includes a nucleic acid sequence encoding amino acids 33-35 of an exemplary tPA pre/prosequence set forth as SEQ ID NO:253. In an additional example, a tPA prosequence includes a nucleic acid sequence encoding amino acids 24-35 of an exemplary tPA pre/prosequence set forth as SEQ ID NO:253. Other tPA prosequences can include amino acids 24-32, 33-35, or 24-35 of allelic variants of tPA pre/prosequences such as set forth in SEQ ID NOS:256 or 257. HGF intron fusion protein-tPA prosequence fusions provided herein can exhibit enhanced cellular expression and secretion of an HGF isoform polypeptide for improved production.

Additionally, an HGF isoform, HGF intron fusion protein-tPA pre/prosequence fusion, HGF intron fusion protein-tPA presequence fusion, and/or an HGF intron fusion protein-tPA prosequence fusion for the improved secretion of an intron fusion protein polypeptide can optionally also include one, two, three, or more fusion tags that facilitate the purification and/or detection of an HGF isoform polypeptide. Generally, a coding sequence for a specific tag can be spliced in frame on the amino or carboxy ends, with or without a linker region, with a coding sequence of a nucleic acid molecule encoding an HGF isoform polypeptide. When fusion is on an amino terminus of a sequence, a fusion tag can be placed between an endogenous or heterologous precursor sequence. In one embodiment a fusion tag, such as a c-myc tag, 8×His tag, or any other fusion tag known to one of skill in the art, can be placed between an HGF isoform endogenous signal sequence and an HGF coding sequence. In another embodiment, a fusion tag can be placed between a heterologous precursor sequence, such as a tPA pre/prosequence, presequence, or prosequence set forth in SEQ ID NO:252, and an HGF isoform coding sequence. In other embodiments, a fusion tag can be placed directly on the carboxy terminus of a nucleic acid encoding an HGF isoform fusion polypeptide sequence. In some instances, an HGF isoform fusion can contain a linker between an endogenous or heterologous precursor sequence and a fusion tag. HGF isoform fusions containing one or more fusion tag(s) provided herein, including HGF intron fusion protein-tPA fusions, can facilitate easier detection and/or purification of an HGF isoform polypeptide for improved production.

b. Chimeric and Synthetic Intron Fusion Polypeptides

Also provided are chimeric HGF fusion polypeptides. A chimeric HGF isoform is a protein encoded by all or part of two or more genes resulting in a polypeptide containing all or part of an encoded HGF sequence operatively linked to another polypeptide. Generally, a chimeric HGF isoform contains all or part of an HGF isoform, including an intron from an HGF intron fusion polypeptide, operatively linked at the N-terminus to another polypeptide or other molecule such that the resulting molecule modulates the activity of a cell surface molecule, particularly an RTK receptor or other angiogenic molecule, including any involved in pathways that participate in the inflammatory response, angiogenesis, neovascularization and/or cell proliferation. Included among these synthetic “polypeptides” are chimeric intron fusion polypeptides in which all or part of an HGF isoform is linked to all or part of an intron fusion protein, such as all or part of any one of the sequences and encoded amino acids as set forth as SEQ ID NOS:36-245. An exemplary chimeric intron fusion polypeptide includes all or part of an HGF isoform linked to an intron 8 portion of a herstatin (see, e.g., SEQ ID NOS:231-245 and encoded amino acids set forth in SEQ ID NOS:216-230). Exemplary herstatins, or intron 8 portions thereof, are set forth in SEQ ID NOS. 201-245. Table 4 below identifies the variations in the intron 8-encoded portion of a herstatin compared to a prominent intron 8 (SEQ ID NO: 216) included between amino acids 341-419 of the prominent herstatin molecule set forth as SEQ ID NO:186. The sequence identifiers (SEQ ID NOS) for exemplary intron 8 and herstatin molecules, including variants of an intron 8 or herstatin, are in parentheses. Other herstatin variants include allelic variants, particularly those with variation in the extracellular domain portion.

TABLE 4
Herstatin variants

| Intron 8 Variant | | Herstatin Variant | |
|---|---|---|---|
| Nucleotide | Amino Acid | Nucleotide | Amino Acid |
| Prominent (231) | Prominent (216) | Prominent (201) | Prominent (186) |
| nt 4 = T (232) | aa 2 = Ser (217) | nt 1036 = T (202) | aa 342 = Ser (187) |
| nt 14 = C (233) | aa 5 = Pro (218) | nt 1046 = C (203) | aa 345 = Pro (188) |
| nt 17 = T (234) | aa 6 = Leu (219) | nt 1049 = T (204) | aa 346 = Leu (189) |
| nt 47 = A (235) | aa 16 = Gln (220) | nt 1079 = A (205) | aa 356 = Gln (190) |
| nt 49 = T (236) | aa 17 = Cys (221) | nt 1081 = T (206) | aa 357 = Cys (191) |
| nt 52 = C (237) | aa 18 = Leu (222) | nt 1084 = C (207) | aa 358 = Leu (192) |
| n 54 = A (238) | aa 18 = Ile (223) | nt 1086 = A (208) | aa 358 = Ile (193) |
| nt 62 = C, T, A | aa 21 = Asp, Ala, | nt 1094 = C, T, A | aa 361 = Asp, Ala, |
| (239) | Val (224) | (209) | Val (194) |
| nt 92 = T (240) | aa 31 = Ile (225) | nt 1124 = T (210) | aa 371 = Ile (195) |
| nt 106 = A (241) | aa 36 = Ile (226) | nt 1138 = A (211) | aa 376 = Ile (196) |
| nt 161 = G (242) | aa 54 = Arg (227) | nt 1193 = G (212) | aa 394 = Arg (197) |
| nt 191 = T (243) | aa 64 = Leu (228) | nt 1223 = T (213) | aa 404 = Leu (198) |
| nt 217 = C or A | aa 73 = His or Asn | nt 1249 = C or A | aa 413 = His or |
| (244) | (229) | (214) | Asn (199) |
| nt 17 = T and | aa 6 = Leu and | nt 1049 = T and | aa 346 = Leu and |
| nt 217 = C or A | aa 73 = His or Asn | nt 1249 = C or A | aa 413 = His or |
| (245) | (230) | (215) | Asn (200) |

The N-terminus portion of an HGF isoform can be linked to a C-terminus (intron-encoded portion) of the synthetic intron fusion protein directly or via a linker, such as a polypeptide linker. For example, linkage can be effected by recombinant expression of a fusion protein where all or part of a nucleic acid encoding an HGF isoform is operatively linked at the 5′ end to all or part of a nucleic acid encoding another intron fusion protein. Linkage can be in the presence of an encoded peptide linker such as any linker described herein or known in the art, or in the presence of a restriction enzyme linker. An HGF isoform encoded polypeptide also can be linked or conjugated to all or part of another polypeptide by chemical linkage such as by using a heterobifunctional cross-linking reagent or any other linkage that can be effected chemically such as is described above for isoform conjugates.

Any suitable linker can be selected so long as the resulting HGF chimeric molecule interacts with a cell surface receptor such as a MET receptor or other cell surface molecule including angiogenic molecules and modulates, typically inhibits, the activity of the cell surface molecule. Linkers can be selected to add a desirable property, such as to increase serum stability, solubility and/or intracellular concentration and to reduce steric hindrance caused by close proximity where one or more linkers is (are) inserted between the N-terminal portion and intron-encoded portion. The resulting molecule is designed or selected to retain the ability to modulate the activity of a cell surface molecule, particularly RTKs or other angiogenic molecules, including any involved in pathways that are involved in inflammatory responses, neovascularization, angiogenesis and cell proliferation and tumor progression.

C. HGF Multimers and Multimerization Domains

Isoform multimers, including HGF multimers, can be covalently-linked, non-covalently-linked, or chemically linked multimers of one or more than one polypeptide to form dimers, trimers, or higher order multimers of the isoforms. The polypeptide components of the multimer can be the same or different. Typically, multimers provided herein are formed between any one or more of the HGF isoforms provided herein, such as for example any set forth in SEQ ID NOS: 10, 12, 14, 18, or 20. In some examples, a multimer can be formed between an HGF isoform and another CSR or ligand isoform. Exemplary CSR isoforms include, but are not limited to, peptides and nucleic acid molecules that encode the polypeptides set forth in SEQ ID NOS: 36-245 and variants thereof.

Multimers of polypeptides can be formed by dimerization, such as via interactions between Fc domains, or they can be covalently joined. Multimerization between two isoform polypeptides can be spontaneous, or can occur due to forced linkage of two or more polypeptides. In one example, multimers can be linked by disulfide bonds formed between cysteine residues on isoforms polypeptides. In an additional example, multimers can be formed between two polypeptides through chemical linkage, such as for example, by using heterobifunctional linkers.

i. Peptide Linkers

Peptide linkers can be used to produce polypeptide multimers. In one example, peptide linkers can be fused to the C-terminal end of a first polypeptide and the N-terminal end of a second polypeptide. This structure can be repeated multiples times such that at least one, preferably 2, 3, 4, or more soluble polypeptides are linked to one another via peptide linkers at their respective termini. For example, a multimer polypeptide can have a sequence Z₁-X-Z₂, where Z₁ and Z₂ are each a sequence of all or part of a CSR or ligand isoform and where X is a sequence of a peptide linker. In some instances, Z₁ and/or Z₂ is a all or part of an isoform polypeptide. In another example, Z₁ and Z₂ are the same or they are different. In another example, the polypeptide has a sequence of Z₁-X-Z₂(-X-Z)ₙ, where “n” is any integer, i.e. generally 1 or 2. Typically, the peptide linker is of sufficient length to so that the resulting polypeptide is soluble. Examples of peptide linkers include glycine serine polypeptides, such s-Gly-Gly-, GGGGG (SEQ ID NO:313), GGGGS (SEQ ID NO:311) or (GGGGS)n, SSSSG (SEQ ID NO:312) or (SSSSG)n.

Linking moieties are described, for example, in Huston et al. (1988) PNAS 85:5879-5883, Whitlow et al. (1993) Protein Engineering 6:989-995, and Newton et al., (1996) Biochemistry 35:545-553. Other suitable peptide linkers include any of those described in U.S. Pat. No. 4,751,180 or U.S. Pat. No. 4,935,233, which are hereby incorporated by reference. A polynucleotide encoding a desired peptide linker can be inserted anywhere in an isoform or at the N- or C-terminus or between a precursor sequence, such as for example, a t-PA preprosequence, in frame, using any suitable conventional technique.

ii. Polypeptide Multimerization Domains

Interaction of two or more polypeptides can be facilitated by their linkage, either directly or indirectly, to any moiety or other polypeptide that are themselves able to interact to form a stable structure. For example, separate encoded polypeptide chains can be joined by multimerization, whereby multimerization of the polypeptides is mediated by a multimerization domain. Typically, the multimerization domain provides for the formation of a stable protein-protein interaction between a first chimeric polypeptide and a second chimeric polypeptide. Chimeric polypeptides include, for example, linkage (directly or indirectly) of a nucleic acid encoding an isoform polypeptide with a nucleic acid encoding a multimerization domain. Homo- or heteromultimeric polypeptides can be generated from co-expression of separate chimeric polypeptides. The first and second chimeric polypeptides can be the same or different.

Generally, a multimerization domain includes any capable of forming a stable protein-protein interaction. The multimerization domains can interact via an immunoglobulin sequence, leucine zipper, a hydrophobic region, a hydrophilic region, or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. patent application Ser. No. 08/399,106. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or typtophan). Compensatory cavities of identical or similar size to the protuberances are optionally created on the interface of the second polypeptide by replacing large amino acid side chains with smaller ones (e.g., alanine or threonine).

A chimeric isoform polypeptide, such as for example an any HGF isoform polypeptide provided herein, can be joined anywhere, but typically via its N- or C-terminus, to the N- or C-terminus of a multimerization domain to ultimately, upon expression, form a chimeric polypeptide.

The resulting chimeric polypeptides, and multimers formed therefrom, can be purified by any suitable method, such as, for example, by affinity chromatography over Protein A or Protein G columns. Where two nucleic acid molecules encoding different chimeric polypeptides are transformed into cells, formation of homo- and heterodimers will occur. Conditions for expression can be adjusted so that heterodimer formation is favored over homodimer formation.

(a) Immunoglobulin Domain

Multimerization domains include those comprising a free thiol moiety capable of reacting to form an intermolecular disulfide bond with a multimerization domain of an additional amino acid sequence. For example, a multimerization domain can include a portion of an immunoglobulin molecule, such as from IgG1, IgG2, IgG3, IgG4, IgA, IgD, IgM, and IgE. Generally, such a portion is an immunoglobulin constant region (Fc). Preparations of fusion proteins fused to various portions of antibody-derived polypeptides (including the Fc domain) has been described, see e.g., Ashkenazi et al. (1991) PNAS 88: 10535; Byrn et al. (1990) Nature, 344:677; and Hollenbaugh and Aruffo, (1992) “Construction of Immnoglobulin Fusion Proteins,” in Current Protocols in Immunology, Suppl. 4, pp. 10.19.1-10.19.11.

Antibodies bind to specific antigens and contain two identical heavy chains and two identical light chains covalently linked by disulfide bonds. The heavy and light chains contain variable regions, which bind the antigen, and constant (C) regions. In each chain, one domain (V) has a variable amino acid sequence depending on the antibody specificity of the molecule. The other domain (C) has a rather constant sequence common among molecules of the same class. The domains are numbered in sequence from the amino-terminal end. For example, the IgG light chain is composed of two immunoglobulin domains linked from N- to C-terminus in the order V_{L}-C_{L}, referring to the light chain variable domain and the light chain constant domain, respectively. The IgG heavy chain is composed of four immunoglobulin domains linked from the N- to C-terminus in the order V_{H}-C_{H}1-C_{H}2-C_{H}3, referring to the variable heavy domain, contain heavy domain 1, constant heavy domain 2, and constant heavy domain 3. The resulting antibody molecule is a four chain molecule where each heavy chain is linked to a light chain by a disulfide bond, and the two heavy chains are linked to each other by disulfide bonds. Linkage of the heavy chains is mediated by a flexible region of the heavy chain, known as the hinge region. Fragments of antibody molecules can be generated, such as for example, by enzymatic cleavage. For example, upon protease cleavage by papain, a dimer of the heavy chain constant regions, the Fc domain, is cleaved from the two Fab regions (i.e. the portions containing the variable regions).

In humans, there are five antibody isotypes classified based on their heavy chains denoted as delta (δ), gamma (γ), mu (μ), and alpha (α) and epsilon (ε), giving rise to the IgD, IgG, IgM, IgA, and IgE classes of antibodies, respectively. The IgA and IgG classes contain the subclasses IgA1, IgA2, IgG1, IgG2, IgG3, and IgG4. Sequence differences between immunoglobulin heavy chains cause the various isotypes to differ in, for example, the number of C domains, the presence of a hinge region, and the number and location of interchain disulfide bonds. For example, IgM and IgE heavy chains contain an extra C domain (C4), that replaces the hinge region. The Fc regions of IgG, IgD, and IgA pair with each other through their Cγ3, Cδ3, and Cα3 domains, whereas the Fc regions of IgM and IgE dimerize through their Cμ4 and Cε4 domains. IgM and IgA form multimeric structures with ten and four antigen-binding sites, respectively.

Immunoglobulin chimeric polypeptides provided herein include a full-length immunoglobulin polypeptide. Alternatively, the immunoglobulin polypeptide is less than full length, i.e. containing a heavy chain, light chain, Fab, Fab2, Fv, or Fc. In one example, the immunoglobulin chimeric polypeptides are assembled as monomers or hetero- or homo-multimers, and particularly as dimer or tetramers. Chains or basic units of varying structures can be utilized to assemble the monomers and hetero- and homo-multimers. For example, an isoform polypeptide can be fused to all or part of an immunoglobulin molecule, including all or part of C_{H}, C_{L}, V_{H}, or V_{L} domain of an immunoglobulin molecule (see. e.g., U.S. Pat. No. 5,116,964). Chimeric isoform polypeptides can be readily produced and secreted by mammalian cells transformed with the appropriate nucleic acid molecule. The secreted forms include those where the isoform polypeptide is present in heavy chain dimers; light chain monomers or dimers; and heavy and light chain heterotetramers where the isoform polypeptide is fused to one or more light or heavy chains, including heterotetramers where up to and including all four variable regions analogues are substituted. In some examples, one or more than one nucleic acid fusion molecule can be transformed into host cells to produce a multimer where the isoforms portions of the multimer are the same or different. In some examples, a non-isoform polypeptide light-heavy chain variable-like domain is present, thereby producing a heterobifunctional antibody. In some examples, a chimeric polypeptide can be made fused to part of an immunoglobulin molecule lacking hinge disulfides, in which non-covalent or covalent interactions of the two polypeptides associate the molecule into a homo- or heterodimer.

(i) Fc Domain

Typically, the immunoglobulin portion of an immunoglobulin chimeric polypeptide fusion, such as fusion with an HGF isoform, includes the heavy chain of an immunoglobulin polypeptide, most usually the constant domains of the heavy chain. Exemplary sequences of heavy chain constant regions for human IgG sub-types are known. For example, for the exemplary heavy chain constant region set forth in SEQ ID NO:296, the CH1 domain corresponds to amino acids 1-98, the hinge region corresponds to amino acids 99-110, the CH2 domain corresponds to amino acids 111-223, and the CH3 domain corresponds to amino acids 224-330.

In one example, an immunoglobulin polypeptide chimeric protein can include the Fc region of an immunoglobulin polypeptide. Typically, such a fusion retains at least a functionally active hinge, CH2 and CH3 domains of the constant region of an immunoglobulin heavy chain. For example, a full-length Fc sequence of IgG1 includes amino acids 99-330 of the sequence set forth in SEQ ID NO:296. Numerous Fc domains are known, including variant Fc domains whose T-cell activity is reduced or eliminated. The precise site at which the linkage is made is not critical: particular sites are well known and can be selected in order to optimize the biological activity, secretion, or binding characteristics of the isoform polypeptide. An exemplary sequence of an Fc domain is set forth in SEQ ID NO:297 or SEQ ID NO:298.

In addition to hIgG1 Fc, other Fc regions also can be included in the isoform polypeptides provided herein. For example, where effector functions mediated by Fc/FcγR interactions are to be minimized, fusion with IgG isotypes that poorly recruit complement or effector cells, such as for example, the Fc of IgG2 or IgG4, is contemplated. Additionally, the Fc fusions can contain immunoglobulin sequences that are substantially encoded by immunoglobulin genes belonging to any of the antibody classes, including, but not limited to IgG (including human subclasses IgG1, IgG2, IgG3, or IgG4), IgA (including human subclasses IgA1 and IgA2), IgD, IgE, and IgM classes of antibodies. Further, linkers can be used to covalently link Fc to another polypeptide to generate an Fc chimera.

Modified Fc domains also are known (see e.g. U.S. Patent Publication No. US 2006/0024298; and International Patent Publication No. WO 2005/063816 for exemplary modifications). In some examples, the Fc region is such that it has altered (i.e. more or less) effector function than the effector function of an Fc region of a wild-type immunoglobulin heavy chain. The Fc regions of an antibody interacts with a number of Fc receptors, and ligands, imparting an array of important functional capabilities referred to as effector functions. Fc effector functions include, for example, Fc receptor binding, complement fixation, and T cell depleting activity (see e.g., U.S. Pat. No. 6,136,310). Methods of assaying T cell depleting activity, Fc effector function, and antibody stability are known in the art. For example, the Fc region of an IgG molecule interacts with the FcγRs. These receptors are expressed in a variety of immune cells, including for example, monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. Recognition of and lysis of bound antibody on target cells by cytotoxic cells that express FcγRs is referred to as antibody dependent cell-mediated cytotoxicity (ADCC). Other Fc receptors for various antibody isotypes include FcεRs (IgE), FcαRs (IgA), and FcμRs (IgM).

Thus, a modified Fc domain can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, ADCC. Such modified Fc regions can contain modifications corresponding to any one or more of G20S, G20A, S23D, S23E, S23N, S23Q, S23T, K30H, K30Y, D33Y, R39Y, E42Y, T44H, V48I, S51E, H52D, E56Y, E561, E56H, K58E, G65D, E67L, E67H, S82A, S82D, S88T, S108G, S1081, K110T, K110E, K110D, A111D, A114Y, A1114L, A1141, 1116D, 1116E, 1116N, 1116Q, E117Y, E117A, K118T, K118F, K118A, and P180L of the exemplary Fc sequence set forth in SEQ ID NO:297, or combinations thereof. A modified Fc containing these mutations can have enhanced binding to an FcR such as, for example, the activating receptor FcγIIIa and/or can have reduced binding to the inhibitory receptor FcγRIIb (see e.g., US 2006/0024298). Fc regions modified to have increased binding to FcRs can be more effective in facilitating the destruction of cancer cells in patients, even when linked with an isoform polypeptide. There are a number of possible mechanisms by which antibodies destroy tumor cells, including anti-proliferation via blockage of need growth pathways, intracellular signaling leading to apopotosis, enhanced down-regulation and/or turnover of receptors, ADCC, and via promotion of the adaptive immune response.

In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035. An exemplary Fc mutein is set forth in SEQ ID NO:299.

In an additional example, an Fc region can be utilized that is modified in its binding to FcRn, thereby improving the pharmacokinetics of an -Fc chimeric polypeptide. FcRn is the neonatal FcR, the binding of which recycles endocytosed antibody from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport.

Typically, a polypeptide multimer is a dimer of two chimeric proteins created by linking, directly or indirectly, two of the same or different isoform polypeptide to an Fc polypeptide. In some examples, a gene fusion encoding an HGF isoform-Fc chimeric protein is inserted into an appropriate expression vector. The resulting Fc chimeric proteins can be expressed in host cells transformed with the recombinant expression vector, and allowed to assemble much like antibody molecules, where interchain disulfide bonds form between the Fc moieties to yield divalent polypeptides. Typically, a host cell and expression system is a mammalian expression system to allow for glycosylation for stabilizing the Fc proteins. Other host cells also can be used where glycosylation at this position is not a consideration.

The resulting chimeric polypeptides containing Fc moieties, and multimers formed therefrom, can be easily purified by affinity chromatography over Protein A or Protein G columns. Where two nucleic acids molecules encoding different chimeric polypeptides are transformed into cells, the formation of heterodimers must be biochemically achieved since chimeric molecules carrying the Fc-domain will be expressed as disulfide-linked homodimers as well. Thus, homodimers can be reduced under conditions that favor the disruption of inter-chain disulfides, but do no effect intra-chain disulfides. Typically, chimeric monomers with different extracellular portions are mixed in equimolar amounts and oxidized to form a mixture of homo- and heterodimers. The components of this mixture are separated by chromatographic techniques. Alternatively, the formation of this type of heterodimer can be biased by genetically engineering and expressing fusion molecules that contain a isoform polypeptide, followed by the Fc-domain of hIgG, followed by either c-jun or the c-fos leucine zippers (see below). Since the leucine zippers form predominantly heterodimers, they can be used to drive the formation of the heterodimers when desired. Chimeric polypeptides containing Fc regions also can be engineered to include a tag with metal chelates or other epitope. The tagged domain can be used for rapid purification by metal-chelate chromatography, and/or by antibodies, to allow for detection of western blots, immunoprecipitation, or activity depletion/blocking in bioassays.

(ii). Protuberances-Into-Cavity (i.e. Knobs and Holes)

Multimers can be engineered to contain an interface between a first chimeric polypeptide and a second chimeric polypeptide to facilitate hetero-oligomerization over homo-oligomerization. Typically, a multimerization domain of one or both of the first and second chimeric polypeptide is a modified antibody fragment such that the interface of the antibody molecule is modified to facilitate and/or promote heterodimerization. In some cases, the antibody molecule is a modified Fc region. Thus, modifications include introduction of a protuberance into a first Fc polypeptide and a cavity into a second Fc polypeptide such that the protuberance is positionable in the cavity to promote complexing of the first and second Fc-containing chimeric polypeptides.

Typically, stable interaction of a first chimeric polypeptide and a second chimeric polypeptide is via interface interactions of the same or different multimerization domain that contains a sufficient portion of a CH3 domain of an immunoglobulin constant domain. Various structural and functional data suggest that antibody heavy chain association is directed by the CH3 domain. For example, X-ray crystallography has demonstrated that the intermolecular association between human IgG1 heavy chains in the Fc region includes extensive protein/protein interaction between CH3 domain whereas the glycosylated CH2 domains interact via their carbohydrate (Deisenhofer et al. (1981) Biochem. 20: 2361). In addition, there are two inter-heavy chain disulfide bonds which are efficiently formed during antibody expression in mammalian cells unless the heavy chain is truncated to remove the CH2 and CH3 domains (King et al. (1992) Biochem. J. 281:317). Thus, heavy chain assembly appears to promote disulfide bond formation rather than vice versa. Engineering of the interface of the CH3 domain promotes formation of heteromultimers of different heavy chains and hinders the assembly of corresponding homomultimers (see e.g., U.S. Pat. No. 5,731,168; International Patent Application WO 98/50431 and WO 2005/063816; Ridgway et al. (1996) Protein Engineering, 9:617-621).

Thus, multimers provided herein can be formed between an interface of a first and second chimeric isoform polypeptide (the first and second polypeptides can be the same or different) where the multimerization domain of the first polypeptide contains at least a sufficient portion of a CH3 interface of an Fc domain that has been modified to contain a protuberance and the multimerization domain of the second polypeptide contains at least a sufficient portion of a CH3 interface of an Fc domain that has been modified to contain a cavity. All or a sufficient portion of a modified CH3 interface can be from an IgG, IgA, IgD, IgE, or IgM immunoglobulin. Interface residues targeted for modification in the CH3 domain of various immunoglobulin molecules are set forth in U.S. Pat. No. 5,731,168. Generally, the multimerization domain is all or a sufficient portion of a CH3 domain derived from an IgG antibody, such as for example, IgG1.

Amino acids targeted for replacement and/or modification to create protuberances or cavities in a polypeptide are typically interface amino acids that interact or contact with one or more amino acids in the interface of a second polypeptide. A first polypeptide that is modified to contain protuberance amino acids include replacement of a native or original amino acid with an amino acid that has at least one side chain which projects from the interface of the first polypeptide and is therefore positionable in a compensatory cavity in an adjacent interface of a second polypeptide. Most often, the replacement amino acid is one which has a larger side chain volume than the original amino acid residue. One of skill in the art can determine and/or assess the properties of amino acid residues to identify those that are ideal replacement amino acids to create a protuberance. Generally, the replacement residues for the formation of a protuberance are naturally occurring amino acid residues and include, for example, arginine (R), phenylalanine (F), tyrosine (Y), or tyrptophan (W). In some examples, the original residue identified for replacement is an amino acid residue that has a small side chain such as, for example, alanine, asparagines, aspartic acid, glycine, serine, threonine, or valine.

A second polypeptide that is modified to contain a cavity is one that includes replacement of a native or original amino acid with an amino acid that has at least one side chain that is recessed from the interface of the second polypeptide and thus is able to accommodate a corresponding protuberance from the interface of a first polypeptide. Often, the replacement amino acid is one which has a smaller side chain volume than the original amino acid residue. One of skill in the art can determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or typtophan.

The CH3 interface of human IgG1, for example, involves sixteen residues on each domain located on four anti-parallel β-strands which buries 1090 A2 from each surface (see e.g., Deisenhofer et al. (1981) Biochemistry, 20:2361-2370; Miller et al., (1990) J. Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. For example, modifications in a CH3 domain to create protuberances or cavities can be replacement of any amino acid corresponding to the interface amino acid Q230, V231, Y232, T233, L234, V246, S247, L248, T249, C250, L251, V252, K253, G254, F255, Y256, K275, T276, T277, P278, V279, L280, D281, G285, S286, F287, F288, L289, Y290, S291, K292, L293, T294, and V295 of the sequence set forth in SEQ ID NO:296. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain. Exemplary of such modifications include, for example, replacement of any amino acid corresponding to the interface amino acid T249, L251, P278, F288, Y290, and K292. Exemplary of amino acid pairs for modification in a CH3 domain interface to create protuberances/cavity interactions include modification of T249 and Y290; and F288 and T277. For example, modifications can include T249Y and Y290T; T249W and Y290A; F288A and T277W; F288W and T277S; and Y290T and T249Y.

In some example, more than one interface interaction can be made. For example, modifications also include, for example, two or more modifications in a first polypeptide to create a protuberance and two or more medications in a second polypeptide to create a cavity. Exemplary of such modifications include, for example, modification of T249Y and F288A in a first polypeptide and modification of T277W and Y290T in a second polypeptide; modification of T277W and F288W in a first polypeptide and modification of T277S and Y290A in a second polypeptide; or modification of F288A and Y290A in a first polypeptide and T249W and T277S in a second polypeptide.

As with other multimerization domains described herein, including all or part of any immunoglobulin molecule or variant thereof, such as an Fc domain or variant thereof, an Fc variant containing CH3 protuberance/cavity modifications can be joined to an isoform polypeptide anywhere, but typically via its N- or C-terminus, to the N- or C-terminus of a first and/or second isoform polypeptide to form a chimeric polypeptide. The linkage can be direct or indirect via a linker. Also, the chimeric polypeptide can be a fusion protein or can be formed by chemical linkage, such as through covalent or non-covalent interactions. Typically, a knob and hole molecule is generated by co-expression of a first isoform polypeptide linked to an Fc variant containing CH3 protuberance modification(s) with a second isoform polypeptide linked to an Fc variant containing CH3 cavity modification(s).

(b). Leucine Zippers

Another method for preparing multimers involves use of a leucine zipper domain. Leucine zippers are peptides that promote multimerization of the proteins in which they are found. Typically, leucine zipper is a term used to refer to a repetitive heptad motif containing four to five leucine residues present as a conserved domain in several proteins. Leucine zippers fold as short, parallel coiled coils, and can be responsible for oligomerization of the proteins of which they form a domain. Leucine zippers were originally identified in several DNA-binding proteins (see e.g., Landschulz et al. (1988) Science 240:1759), and have since been found in a variety of proteins. Among the known leucine zippers are naturally occurring peptides and derivatives thereof that dimerize or trimerize. Recombinant chimeric proteins containing an isoform polypeptide linked, directly or indirectly, to a leucine zipper peptide can be expressed in suitable host cells, and the polypeptide multimer that forms can be recovered from the culture supernatant.

Leucine zipper domains fold as short, parallel coiled coils (O'Shea et al. (1991) Science, 254:539). The general architecture of the parallel coiled coil has been characterized, with a “knobs-into-holes” packing, first proposed by Crick in 1953 (Acta Crystallogr., 6:689). The dimer formed by a leucine zipper domain is stabilized by the heptad repeat, designated (abcdefg)n (see e.g., McLachlan and Stewart (1978) J. Mol. Biol. 98:293), in which residues a and d are generally hydrophobic residues, with d being a leucine, which lines up on the same face of a helix. Oppositely-charged residues commonly occur at positions g and e. Thus, in a parallel coiled coil formed from two helical leucine zipper domains, the “knobs” formed by the hydrophobic side chains of the first helix are packed into the “holes” formed between the side chains of the second helix.

The leucine residues at position d contribute large hydrophobic stabilization energies, and are important for dimer formation (Krystek et al. (1991) Int. J. Peptide Res. 38:229). Hydrophobic stabilization energy provides the main driving force for the formation of coiled coils from helical monomers. Electrostatic interactions also contribute to the stoichiometry and geometry of coiled coils.

(i). fos and jun

Two nuclear transforming proteins, fos and jun, exhibit leucine zipper domains, as does the gene product of the murine proto-oncogene, c-myc. The leucine zipper domain is necessary for biological activity (DNA binding) in these proteins. The products of the nuclear oncogenes fos and jun contain leucine zipper domains that preferentially form a heterodimer (O'Shea et al. (1989) Science, 245:646; Turner and Tijian (1989) Science, 243:1689). For example, the leucine zipper domains of the human transcription factors c-jun and c-fos have been shown to form stable heterodimers with a 1:1 stoichiometry (see e.g., Busch and Sassone-Corsi (1990) Trends Genetics, 6:36-40; Gentz et al., (1989) Science, 243:1695-1699). Although jun-jun homodimers also have been shown to form, they are about 1000-fold less stable than jun-fos heterodimers.

Thus, typically an isoform polypeptide multimer provided herein is generated using a jun-fos combination. Generally, the leucine zipper domain of either c-jun or c-fos is fused in frame at the C-terminus of an isoform of a polypeptide by genetically engineering fusion genes. Exemplary sequences of a c-jun or c-fos leucine zipper domain is set forth in SEQ ID NOS: 300 and 301, respectively. In some instances, a sequence of a leucine zipper can be modified, such as by the addition of a cysteine residue to allow formation of disulfide bonds, or the addition of a tyrosine residue at the C-terminus to facilitate measurement of peptide concentration. Exemplary sequences of a modified c-jun or c-fos leucine zipper domain are set forth in SEQ ID NOS: 302 and 303, respectively. In addition, the linkage of an isoform polypeptide with a leucine zipper can be direct or can employ a flexible linker domain, such as for example a hinge region of IgG, or other polypeptide linkers of small amino acids such as glycine, serine, threonine, or alanine at various lengths and combinations. In some instances, separation of a leucine zipper from the C-terminus of an encoded polypeptide can be effected by fusion with a sequence encoding a protease cleavage sites, such as for example, a thrombin cleavage site. Additionally, the chimeric proteins can be tagged, such as for example, by a 6XHis tag, to allow rapid purification by metal chelate chromatography and/or by epitopes to which antibodies are available, such as for example a myc tag, to allow for detection on western blots, immunoprecipitation, or activity depletion/blocking bioassays.

(ii). GCN4

A leucine zipper domain also occurs in a nuclear protein that functions as a transcriptional activator of a family of genes involved in the General Control of Nitrogen (GCN4) metabolism in S. cerevisiae. An exemplary sequence of the GCN4 leucine zipper domain is set forth in SEQ ID NO: 304. The protein is able to dimerize and bind promoter sequences containing the recognition sequence for GCN4, thereby activating transcription in times of nitrogen deprivation. Amino acid substitutions in the a and d residues of a synthetic peptide representing the GCN4 leucine zipper domain, change the oligomerization properties of the leucine zipper domain. For example, when all residues at position a are changed to isoleucine, the leucine zipper still forms a parallel dimer. When, in addition to this change, all leucine residues at position d also are changed to isoleucine, the resultant peptide spontaneously forms a trimeric parallel coiled coil in solution. Exemplary sequences of trimer and tetramer forms of a GCN4 leucine zipper domain are set forth in SEQ ID NOS: 305 and 306, respectively.

(c). Other Multimerization Domains

Other multimerization domains are known to those of skill in the art and are any that facilitate the protein-protein interaction of two or more polypeptides that are separately generated and expressed. Examples of other multimerization domains that can be used to provide protein-protein interactions between or among polypeptides include, but are not limited to, the bamase-barstar module (see e.g., Deyev et al., (2003) Nat. Biotechnol. 21:1486-1492); selection of particular protein domains (see e.g., Terskikh et al., (1997) PNAS 94: 1663-1668 and Muller et al., (1998) FEBS Lett. 422:259-264); selection of particular peptide motifs (see e.g., de Kruif et al., (1996) J. Biol. Chem. 271:7630-7634 and Muller et al., (1998) FEBS Lett. 432: 45-49); and the use of disulfide bridges for enhanced stability (de Kruif et al., (1996) J. Biol. Chem. 271:7630-7634 and Schmiedl et al., (2000) Protein Eng. 13:725-734). Exemplary of another type of multimerization domain is one where multimerization is facilitated by protein-protein interactions between different subunit polypeptides, such as is described below for PKA/AKAP interaction.

R/PKA-AD/AKAP

Multimeric polypeptides also can be generated utilizing protein-protein interactions between the regulatory (R) subunit of cAMP-dependent protein kinase (PKA) (see e.g., SEQ ID NO:307 or SEQ ID NO:309) and the anchoring domains (AD) of A kinase anchor proteins (AKAPs, see e.g., Rossi et al., (2006) PNAS 103:6841-6846) (see e.g., see e.g., SEQ ID NO:308 or SEQ ID NO:310). Two types of R subunits (RI and RII) are found in PKA, each with an α and β isoform. The R subunits exist as dimers, and for RII, the dimerization domain resides in the 44 amino-terminal residues. AKAPs, via the interaction of their AD domain, interact with the R subunit of PKA to regulate its activity. AKAPs bind only to dimeric R subunits. For example, for human RIIα, the AD binds to a hydrophobic surface formed from the 23 amino-terminal residues.

d. Methods of Generating and Cloning HGF Fusions

The methods by which DNA sequences may be obtained and linked to provide the DNA sequence encoding the fusion protein are well known in the field of recombinant DNA technology. DNA for a sequence to be fused to an HGF isoform including, but not limited to, a sequence of an HGF isoform, a precursor signal sequence, a fusion tag, another isoform or intron-encoded portion thereof, or any other desired sequence can be generated by various methods including: synthesis using an oligonucleotide synthesizer; isolation from a target DNA such as from an organism, cell, or vector containing the sequence by appropriate restriction enzyme digestion; or can be obtained from a target source by PCR of genomic DNA with the appropriate primers. In a PCR method, primers directed against a target sequence, such as an HGF isoform sequence, can be engineered that contain sequences for small epitope tags, such as a myc tag, His tag, or other small epitope tag, and/or any other additional DNA sequence such as a restriction enzyme linker sequence or a protease cleavage site sequence such that the entire PCR sequence is incorporated into a target nucleic acid sequence upon PCR amplification. In an exemplary embodiment, the primer can introduce restriction enzyme sites into an HGF isoform sequence, or other target sequence, to facilitate the cloning of the sequence into a vector.

In one example, HGF isoform fusion sequences can be generated by successive rounds of ligating DNA target sequences, amplified by PCR, into a vector at engineered recombination sites. For example, a nucleic acid sequence for an HGF isoform, fusion tag, homologous or heterologous precursor sequence, or other desired nucleic acid sequence can be PCR amplified using primers that hybridize to opposite strands and flank the region of interest in a target DNA. Cells or tissues or other sources known to express a target DNA molecule, or a vector containing a sequence for a target DNA molecule, can be used as a starting product for PCR amplification events. The PCR amplified product can be subcloned into a vector for further recombinant manipulation of a sequence, such as to create a fusion with another nucleic acid sequence already contained within a vector, or for the expression of a target molecule.

PCR primers used in the PCR amplification also can be engineered to facilitate the operative linkage of nucleic acid sequences. For example, non-template complementary 5′ extension can be added to primers to allow for a variety of post-amplification manipulations of the PCR product without significant effect on the amplification itself. For example, these 5′ extensions can include restriction sites, promoter sequences, sequences for epitope tags, etc. In one example, for the purpose of creating a fusion sequence, sequences that can be incorporated into a primer include, for example, a sequence encoding a myc epitope tag or other small epitope tag, such that the amplified PCR product effectively contains a fusion of a nucleic acid sequence of interest with an epitope tag.

In another example, incorporation of restriction enzyme sites into a primer can facilitate subcloning of the amplification product into a vector that contains a compatible restriction site, such as by providing sticky ends for ligation of a nucleic acid sequence. Subcloning of multiple PCR amplified products into a single vector can be used as a strategy to operatively link or fuse different nucleic acid sequences. Examples of restriction enzyme sites that can be incorporated into a primer sequence can include, but are not limited to, an Xho I restriction site, an Nhe I restriction site, a Not I restriction site, an EcoR I restriction site, or an Xba I restriction site. Other methods for subcloning of PCR products into vectors include blunt end cloning, TA cloning, ligation independent cloning, and in vivo cloning.

The creation of an effective restriction enzyme site into a primer facilitates the digestion of the PCR fragment with a compatible restriction enzyme to expose sticky ends, or for some restriction enzyme sites, blunt ends, for subsequent subcloning. There are several factors to consider in engineering a restriction enzyme site into a primer so that it retains its compatibility for a restriction enzyme. First, the addition of 2-6 extra bases upstream of an engineered restriction site in a PCR primer can greatly increase the efficiency of digestion of the amplification product. Other methods that can be used to improve digestion of a restriction enzyme site by a restriction enzyme include proteinase K treatment to remove any thermostable polymerase that can block the DNA, end-polishing with Klenow or T4 DNA polymerase, and/or the addition of spermidine. An alternative method for improving digestion efficiency of PCR products also can include concatamerization of the fragments after amplification. This is achieved by first treating the cleaned up PCR product with T4 polynucleotide kinase (if the primers have not already been phosphorylated). The ends may already be blunt if a proofreading thermostable polymerase such as Pfu was used or the amplified PCR product can be treated with T4 DNA polymerase to polish the ends if a non-proofreading enzyme such as Taq is used. The PCR products can be ligated with T4 DNA ligase. This effectively moves the restriction enzyme site away from the end of the fragments and allows for efficient digestion.

Prior to subcloning of a PCR product containing exposed restriction enzyme sites into a vector, such as for creating a fusion with a sequence of interest, it is sometimes necessary to resolve a digested PCR product from those that remain uncut. In such examples, the addition of fluorescent tags at the 5′ end of a primer can be added prior to the PCR. This allows for identification of digested products since those that have been digested successfully will have lost the fluorescent label upon digestion.

In some instances, the use of amplified PCR products containing restriction sites for subsequent subcloning into a vector for the generation of a fusion sequence can result in the incorporation of restriction enzyme linker sequences in the fusion protein product. Generally such linker sequences are short and do not impair the function of a polypeptide so long as the sequences are operatively linked.

The nucleic acid molecule encoding an isoform fusion protein can be provided in the form of a vector which comprises the nucleic acid molecule. One example of such a vector is a plasmid. Many expression vectors are available and known to those of skill in the art and can be used for expression of an HGF isoform, including isoform fusions. The choice of expression vector can be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

2. Targeting Agent/Targeting Agent Conjugates

HGF polypeptide isoforms also can be provided as conjugates between the isoform and another agent. The conjugate can be used to target to a receptor with which the isoform interacts and/or to another targeted receptor for delivery of the isoform. Such conjugates include linkage of an HGF isoform to a targeted agent and/or targeting agent. Conjugates can be produced by any suitable method including by expression of fusion proteins in which, for example, DNA encoding a targeted agent or targeting agent, with or without a linker region, is operatively linked to DNA encoding an HGF isoform. Protein conjugates also can be produced by chemical coupling of an HGF isoform polypeptide, typically through disulfide bonds between cysteine residues present in or added to the components, or through amide bonds or other suitable bonds, such as by using heterobifunctional cross-linking reagents such as those provided herein or known in the art. Ionic or other linkages also are contemplated.

Conjugates can contain one or more HGF isoforms linked, either directly or via a linker, to one or more targeted agents: (HGF isoform)n, (L)q, and (targeted agent)m in which at least one HGF isoform is linked directly or via one or more linkers (L) to at least one targeted agent. Such conjugates also can be produced with any portion of an HGF isoform sufficient to bind to a target, such as a target cell type for treatment. Any suitable association among the elements of the conjugate and any number of elements where n, and m are integers greater than 1 and q is zero or any integer greater than 1, is contemplated as long as the resulting conjugates interact with a targeted cell surface receptor, such as MET, or to a targeted cell type.

Examples of a targeted agent include drugs and other cytotoxic molecules such as toxins that act at or via the cell surface and those that act intracellularly. Examples of such moieties include radionuclides, radioactive atoms that decay to deliver, e.g., ionizing alpha particles or beta particles, or X-rays or gamma rays, that can be targeted when coupled to an HGF isoform. Other examples include chemotherapeutics that can be targeted by coupling with an isoform. For example, geldanamycin targets proteosomes. An isoform-geldanamycin molecule can be directed to intracellular proteosomes, degrading the targeted isoform and liberating geldanamycin at the proteosome. Other toxic molecules include toxins, such as ricin, saporin and natural products from conches or other members of phylum mollusca. Another example of a conjugate with a targeted agent is an HGF isoform coupled, for example as a protein fusion, with an antibody or antibody fragment. For example, an isoform can be coupled to an Fc fragment of an antibody that binds to a specific cell surface marker to induce killer T cell activity in neutrophils, natural killer cells, and macrophages. A variety of toxins are well known to those of skill in the art.

Conjugates also can contain one or more HGF isoforms linked, either directly or via a linker, to one or more targeting agents: (HGF isoform)n, (L)q, and (targeting agent)m in which at least one HGF isoform is linked directly or via one or more linkers (L) to at least one targeting agent. Any suitable association among the elements of the conjugate and any number of elements where n, and m are integers greater than 1 and q is zero or any integer greater than 1, is contemplated as long as the resulting conjugates interacts with a target, such as a targeted cell type.

Targeting agents include any molecule that targets an HGF isoform to a target such as a particular tissue or cell type or organ. Examples of targeting agents include cell surface antigens, cell surface receptors, proteins, lipids and carbohydrate moieties on the cell surface or within the cell membrane, molecules processed on the cell surface or secreted, and other extracellular molecules. Molecules useful as targeting agents include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting agents include ligands for receptors, such as proteinaceous and small molecule ligands, and antibodies and binding proteins, such as antigen-binding proteins.

Alternatively, the HGF isoform, which specifically interacts with a particular receptor, receptors, or other molecule, is the targeting agent and is linked to targeted agent, such as a toxin, drug or nucleic acid molecule. The nucleic acid molecule can be transcribed and/or translated in the targeted cell or it can be a regulatory nucleic acid molecule.

The HGF isoform can be linked directly to the targeted agent (or targeting agent) or via a linker. Linkers include peptide and non-peptide linkers and can be selected for functionality, such as to relieve or decrease steric hindrance caused by proximity of a targeted agent or targeting agent to an HGF isoform and/or increase or alter other properties of the conjugate, such as the specificity, toxicity, solubility, serum stability and/or intracellular availability and/or to increase the flexibility of the linkage between an HGF isoform and a targeted agent or targeting agent. Linkage can also be by chemical cross-linking such as by using a heterobifunctional cross-linker as described herein. Examples of linkers and conjugation methods are known in the art (see, for example, WO 00/04926). HGF isoforms also can be targeted using liposomes and other such moieties that direct delivery of encapsulated or entrapped molecules.

3. Peptidomimetic Isoforms

Also provided are “peptidomimetic” isoforms in which one or more bonds in the peptide backbone (or other bond(s)) is (are) replaced by a bioisostere or other bond such that the resulting polypeptide peptidomimetic has improved properties, such as resistance to proteases, compared to the unmodified form.

### H. Methods for Altering Serum Half-Life and Other Therapeutic Properties

Methods are provided herein for increasing the serum half-life, stability, solubility and/or reducing immunogenicity of a polypeptide. Increasing the carbohydrate content of a protein can affect these properties. Methods for increasing the carbohydrate content include the introduction of one or more consensus sites for glycosylation into the target protein. Carbohydrate content also can be increased by altering the pattern, or spacing, of existing glycosylation sites within a target protein. Introduction of consensus glycosylation sites or alteration of consensus glycosylation sites can be accomplished by amino acid substitution or by addition of a polypeptide sequence containing consensus sites for glycosylation. A polypeptide sequence containing consensus sites for glycosylation can be fused to either the amino- or carboxy-terminus of the target protein, or alternatively, can be engineered to occur within the target protein to thereby increase its carbohydrate content.

Provided herein are methods for increasing carbohydrate content of a polypeptide and polypeptide products that have increased carbohydrate content. In particular, fusion proteins containing all or a portion of a CD45 protein are provided. The portion of CD45 is selected to include one or more, generally two, three, four or more glycosylation sites. This portion is fused to a protein, at the N-terminus, C-terminus or internally. The site for insertion is selected so that the protein retains an activity, particularly a therapeutic activity. Insertion of the CD45 fragment should not substantially alter such activity and is selected so that at least, 1%, 2%, 5%, 10%, 20%, 30%, 40%, 50%, 60%, 70%, 80%, 90%, 95%, 96%, 97%, 98%, 99% or more of the activity is retained. The particular amount of activity retained is dependent upon the protein whose carbohydrate content is being increased and its intended use. If necessary, the amount can be empirically determined. Some proteins, such as proteases, are so active that retention of only 1% of the activity is still sufficient for many purposes. Other proteins may only tolerate a 10% loss of activity before their purpose is compromised. Assays to assess activity are available for most, if not all therapeutic proteins and other active proteins, or can be developed.

1. N-Linked and O-Linked Glycosylation

Proteins can be modified in any way that increases glycosylation. For example, they can be modified by adding N-linked or O-linked glycosylation sites. O-linked glycosylation occurs by addition of a monosaccharide, such as N-acetylgalactosamine (GalNac), to the hydroxyl group of a Ser or Thr residue in the target protein. In collagens, galactose is added to the hydroxyl group of hydroxylysine. Glycosyltransferases subsequently attach additional carbohydrate moieties to the modified residue to form a mature O-glycan. O-linked oligosaccharides typically contain one to four sugar residues. O-linked glycosylation occurs at sites defined by protein secondary structures, such as an extended beta turn. N-linked glycosylation occurs by addition of a 14-residue oligosaccharide, N-acetylglucosamine (GlcNAc), to the amide nitrogen of an Asn residue with a consensus motif, Asn-X-Ser/Thr, where X is any amino acid with the exception of Pro. Glycosyltransferases subsequently alter the attached oligosaccharide to form a mature N-glycan. N-linked oligosaccharides contain mannose, N-acetylglucosamine and typically have several branches of carbohydrates, each terminating with a negatively charged sialic acid residue. Protein secondary structure can affect the availability of consensus sites as targets for glycosylation.

Glycosylation reactions occur within the lumina of cell organelles involved in the secretory pathway, including the endoplasmic reticulum (ER) and the cis-, medial-, and trans-Golgi cisternae. Signal sequences can target the nascent polypeptides to the ER. Signal sequences can be present in the wild-type protein or can be engineered through recombinant DNA techniques by fusion of a nucleotide sequence encoding the signal peptide to the nucleotide sequence encoding the target protein.

2. Effects of Glycosylation

Glycosylation can increase serum-half-life of polypeptides by increasing the stability and solubility, and reducing the immunogenicity of a protein. Glycosylation can increase the stability of proteins by reducing the proteolysis of the protein. Glycosylation can protect the protein from thermal degradation, exposure to denaturing agents, damage by oxygen free radicals, and changes in pH. Glycosylation also can allow the target protein to evade clearance mechanisms that can involve binding to other proteins, including cell surface receptors. Carbohydrate moieties that contain sialic acid can affect the solubility of a protein. The sialic acid moieties are highly hydrophilic and can shield hydrophobic residues of the target protein. This decreases aggregation and precipitation of the target protein. Decreased aggregation also aids in the prevention of the immune response against the target protein. Carbohydrates can furthermore shield immunogenic sequences from the immune system. The volume of space occupied by the carbohydrate moieties can decrease the available surface area that is surveyed by the immune system. These properties lead to the reduction in immunogenicity of the target protein.

3. Therapeutic Uses for Glycosylation

Increasing the serum half-life of proteins can improve their potential for use as therapeutics. Rapid clearance of therapeutic proteins by the body decreases the efficacy of treatments and increases the number of injections needed by the patient. Increasing the serum half-life through methods, such as enhancing the glycosylation of the therapeutic protein, can ameliorate the need for frequent injections. Other effects of glycosylation, such as solubility and decreased immunogenicity of the target protein, are desirable characteristics for therapeutic proteins. Increased solubility can increase the options for suitable compositions for delivery of the therapeutic protein and can enhance the ability of the therapeutic protein to reach the target tissue once inside the body. Decreasing the immunogenicity of the protein can decrease likelihood adverse immune reactions.

Examples of therapeutic proteins that can be engineered to increase their glycosylation include, but are not limited to, growth factors, antibodies, cytokines, such as tumor necrosis factors and interleukins, and cytotoxic agents and other agents disclosed herein and known to those of skill in the art. Such agents include, but are not limited to, tumor necrosis factor, α-interferon, β-interferon, nerve growth factor, platelet derived growth factor, tissue plasminogen activator; or biological response modifiers such as, for example, lymphokines, interleukin-I (IL-1), interleukin-2 (IL-2), interleukin-6 (IL-6), granulocyte macrophage colony stimulating factor (GM-CSF), granulocyte colony stimulating factor (G-CSF), erythropoietin (EPO), pro-coagulants such as tissue factor and tissue factor variants, pro-apoptotic agents such FAS-ligand, fibroblast growth factors (FGF), nerve growth factor and other growth factors.

4. Use of CD45 for Altering Serum Half-Life

CD45 is a transmembrane protein tyrosine phosphatase that contains an extracellular domain that is heavily glycosylated, a single transmembrane domain, and an intracellular domain containing tandemly duplicated phosphatase domains. Fusions of a target protein to the extracellular domain of CD45, or fragments thereof, can be engineered to alter, particularly increase, the serum half-life of the target protein by increasing the overall carbohydrate content of the recombinant protein. Methods are provided herein for the use of CD45 extracellular domain, or fragments thereof, for the production of CD45 fusion proteins.

An exemplary full-length CD45 polypeptide is provided herein as SEQ ID NO: 272 encoded by the nucleic acid sequence set forth as SEQ ID NO: 271. An allelic variant of CD45 can contain one or more nucleotide changes compared to SEQ ID NO: 271 or one or more amino acid changes compared to SEQ ID NO: 272. Allelic variation can occur in any one or more of the exon or intron sequences of a CD45 gene. Nucleic acids encoding CD45 proteins and the encoded CD45 polypeptides can include allelic variants of CD45. An exemplary CD45 allelic variant can include any one or more nucleotide changes as set forth in SEQ ID NO: 273 or any one or more amino acid changes as set forth in SEQ ID NO: 274. Furthermore, where CD45 is added to increase carbohydrate content, variation and modifications can be introduced that do affect the glycoslyation sites and/or that add additional glycosylation sites. Hence variants of the CD45 polypeptide disclosed herein and those known to those of skill in the art can be employed. Such variants can have 40%, 50%, 60%, 70%, 80%, 90%, 95%, 96%, 97%, 98%, 99% identity to the CD45 polypeptides disclosed herein or to allelic and species variants thereof.

a. CD45 Function

CD45 is expressed on all nucleated cells of hematopoietic origin and functions in lymphocyte receptor activation and development. The intracellular phosphatase domain of CD45 modulates the activity of Src family protein tyrosine kinases, such as Lck and Fyn, by removal of an inhibitory phosphate on the peptide activation loop that inhibits the kinase activity by blocking substrate binding. Activation of these kinases contributes to T cell activation, T cell development, and B cell development via B cell receptor (BCR) activation.

b. CD45 Dimerization And Glycosylation

The activity of CD45 can be controlled by dimerization of the receptor. Dimerization of the extracellular region can lead to inactivation of the intracellular phosphatase activity. The inhibition occurs through reciprocal interaction of an inhibitory structure of one CD45 protein with the phosphatase domain of another CD45 protein. The CD45 dimer represents the inactive form of the receptor, whereas monomeric forms of CD45 represent an active, or “primed”, state of the receptor, where the active phosphatase is poised to respond to lymphocyte activation. Differences in receptor dimerization can be achieved through changes in the carbohydrate content of the CD45 extracellular region. Increased glycosylation causes an increase in the monomeric form of CD45, leading to increased phosphatase activity. Glycosylation of the extracellular region also promotes the binding of lectins, such as CD22 and galectin-1, to the cell surface, though these proteins bind generally to T-cell glycoproteins and do not appear to be involved in signaling through CD45 phosphatase domain.

Changes in glycosylation of CD45 can be achieved through alternative splicing of exons encoding glycosylated domains of the receptor. Exons 4, 5, and 6 (named A, B, and C domains, respectively) encode a polypeptide region near the N-terminus of the protein that is heavily O-glycosylated with variable sialic acid modification. Alternative splicing of the 4, 5, and 6 exons produces different isoforms of CD45. The extracellular regions of the isoforms vary in size, shape, and charge in large part due to differences in carbohydrate content. The CD45 isoform RO that lacks all three domains is approximately 180 kDa in size whereas the CD45 isoform RABC that includes the A, B, and C, domains is approximately 220-240 kDa. The RO and RABC isoforms of CD45 are expressed differentially depending on the cell type, developmental stage, and cell activation state. For example, activated T cells express high levels of the RABC isoform on the first day of stimulation and then gradually switches expression to the RO isoform as activation decreases. For another example, naïve T cells, which are primed for activation, express high levels of the RABC isoform whereas memory T cells, which have lower tyrosine kinase activation, express the RO isoform. Other alternatively spliced CD45 variants encode isoforms that include different combinations of the A, B, and C domains. For example, a 210 kDa isoform contains either A and B or B and C domains and a 200 kDa isoform contains the B domain.

The remainder of the extracellular domain also is heavily glycosylated. This region contains a cysteine rich domain (d1) followed by three fibronectin type III repeat domains (d2, d3, and d4). Glycosylation in this region is predominantly N-linked glycosylation. The N-linked conjugates are tetra- and triantennary complex-type carbohydrate chains that contain poly(N-acetyllactosamine) groups and α-2,6 sialic acid residues. The N-linked glycosylation of these domains contributes to binding of CD22 of B cells, serum mannan-binding protein, and the glucosidase II lectin found in the endoplasmic reticulum. Binding of these proteins to CD45 can contribute to cell adhesion, thymocyte maturation, and alteration of carbohydrate content, respectively.

TABLE 5
CD45 Extracellular Region-Domains
and Potential Glycosylation Sites

| NT | AA | CD45 | Domain | Potential |
|---|---|---|---|---|
| SEQ | SEQ | Extracellular | Location | Glycosylation |
| ID | ID | Domains | (human CD45) | Sites |
| 280 | 281 | Extracellular | 32-575 | (see below, and N197) |
| | | Domain | | |
| 282 | 283 | A | 32-97 | O-linked: various |
| 284 | 285 | B | 98-144 | undefined S/T residues |
| 286 | 287 | C | 145-192 | in domain |
| | | | | N-linked: N78, N90, |
| | | | | N95, N184, N190 |
| 288 | 289 | d1 - Cysteine | 218-299 | N-linked: N232, N260, |
| | | rich | | N270, N276 |
| 290 | 291 | d2 - Fibronectin | 300-388 | N-linked: N335, N378 |
| | | type III | | |
| 292 | 293 | d3 - Fibronectin | 389-481 | N-linked: N419, N468 |
| | | type III | | |
| 294 | 295 | d4 - Fibronectin | 482-572 | N-linked: N488, N529 |
| | | type III | | |

c. CD45 Fusion Proteins

CD45 fusion proteins contain a polypeptide and a CD45 protein fragment and combinations of fragments thereof. The CD45 fragment is derived from the extracellular region of CD45 as outlined in the Table 5 above and set forth in SEQ ID NOS:281, 283, 285, 287, 289, 291, 293, 295, and variants thereof. Provided herein are CD45 fusion proteins that contain a cell surface receptor (CSR) isoform and a CD45 protein fragment derived from the extracellular region of CD45, and combinations of fragments thereof. In a further embodiment, a CD45 fusion protein contains, a CSR isoform and a CD45 protein fragment, or combinations of fragments thereof, containing one or more glycosylation sites. Exemplary CD45 protein fragments include, but are not limited to, peptides set forth in SEQ ID NOS: 281, 283, 285, 287, 289; 291, 293, 295, and variants thereof, including allelic and species variants, and any having at least or at least about 50%, 60%, 70%, 80%, 90%, 95%, 96%, 97%, 98%, 99% or more sequence identity to these CD45 proteins. Exemplary CD45 protein fragments are encoded by nucleic acid molecules that contain the sequence of nucleotides set forth in SEQ ID NOS: 280, 282, 284, 286, 288, 290, 292, 294, and variants, including species and allelic variants, thereof. Exemplary CSR isoforms include, but are not limited to, peptides and nucleic acid molecules that encode the polypeptides set forth in SEQ ID NOS: 36-245 and variants thereof. In a further embodiment, nucleic acid molecules encoding a CD45 fusion protein contains a CSR isoform and a CD45 protein, or fragments thereof, and are provided herein. Variants of peptide sequences set forth in SEQ ID NOS: 281, 283, 285, 287, 289, 291, 293, and 295 and nucleic acid sequences set forth in SEQ ID NOS: 280, 282, 284, 286, 288, 290, 292, and 294 are provided as set forth in SEQ ID NOS: 273 and 274 respectively.

Provided herein are CD45 fusion proteins containing a ligand isoform and a CD45 protein, or fragment thereof. Provided herein are CD45 fusion proteins containing a ligand isoform and a CD45 protein fragment, or combinations of fragments thereof, derived from the extracellular region of CD45. In a further embodiment, a CD45 fusion protein containing a ligand isoform and a CD45 protein fragment, or combinations of fragments thereof, containing one or more putative glycosylation sites. Exemplary protein fragments include, but are not limited to peptides set forth in SEQ ID NOS: 281, 283, 285, 287, 289, 291, 293, 295, and variants thereof. Exemplary CD45 protein fragments are encoded by nucleic acids set forth in SEQ ID NOS: 280, 282, 284, 286, 288, 290, 292, 294, and variants thereof. Exemplary ligand isoforms include, but are not limited to peptides and the nucleic acid molecules that encode the polypeptides set forth in SEQ ID NOS: 10-14, 18, 20, or variants thereof. In a further embodiment, nucleic acid molecules encoding a CD45 fusion protein contains a ligand isoform and a CD45 protein, or fragments thereof, and are provided herein.

CD45 fusion proteins can contain combinations of entire CD45 protein fragments, or portions thereof, of peptides set forth in SEQ ID NOS: 280-295 and variants thereof. Allelic variants of CD45 also include species variants. CD45 is present in multiple species besides human such as, but not limited to, other mammals, birds, fish, reptiles, amphibians and insects. Exemplary sequences for species variants of CD45 include, but are not limited to, chimpanzee, mouse, rat, dog, and chicken, which are set forth in SEQ ID NOS: 275-279.

In other embodiments, a CD45 fusion protein contains a biologically active and/or therapeutically active variant of a CSR isoform, and a CD45 protein, or fragments thereof. In other embodiments, a CD45 fusion protein contains a biologically active and/or therapeutically active variant of a ligand isoform, and a CD45 protein, or fragments thereof.

Vectors containing the nucleic acid molecules encoding CD45-CSR isoform or CD45-ligand isoform fusion proteins are provided as are cells containing the vectors or nucleic acid molecules. Among the nucleic acid molecules provided are those that contain an intron and an exon, where the intron contains a stop codon; the nucleic acid molecule encodes an open reading frame that spans an exon intron junction; and the open reading frame terminates at the stop codon in the intron. The intron can encode one or more amino acids of the encoded polypeptide or the codon can be a first codon (and possibly the only codon) in the intron.

A non-exhaustive list of protein isoforms that can be fused to CD45, or fragments thereof, includes but is not limited to, CSR isoforms and ligand isoforms containing polypeptides and the nucleic acids encoding the polypeptides set forth in SEQ ID NOS: 10-14, 18, 20 and 36-245, including fragments and variants thereof.

d. Conjugates of CD45 Fusion Proteins

Nucleic acid molecules that can be joined to an CD45 fusion protein include, but are not limited to, for example, promoter sequences designed to facilitate intracellular protein expression, secretion sequences designed to facilitate protein secretion, regulatory sequences for regulating transcription and translation, molecules that regulate the serum stability of an encoded polypeptide such as an Fc portion of an immunoglobulin, and other polypeptide-encoding nucleic acid molecules such as those encoding a targeted agent or targeting agent, or those encoding all or part of another ligand or cell surface receptor intron fusion protein. The fusion sequence can be a component of an expression vector, or it can be part of a nucleic acid sequence that is inserted into an expression vector. In one embodiment, the CD45 fusion proteins can contain peptide sequence tags employed for detection and/or isolation of the fusion proteins by techniques known in the art, such as by western blotting, fluorescence microscopy, immunohistochemistry, immunoprecipitation, and column purification. Exemplary sequence tags include, but are not limited to a myc tag, Poly-His tag, GST tag, Flag tag, fluorescent or luminescent moiety such as GFP or luciferase, or any other epitope or fusion tag known to one of skill in the art. In another embodiment, the CD45 fusion proteins additionally contain signal sequence peptides employed to enable and/or to enhance secretion of the fusion protein. Exemplary signal sequence peptides include, but are not limited to, tPA pre/pro signal sequences as disclosed herein (see, e.g., SEQ ID NOS: 256-265). Additional conjugates, such as targeting agent conjugates, crosslinking agents, polypeptide linkers and fusions to all or part of another polypeptide, as described in Section G, can be applied to CD45 fusion proteins.

e. Therapeutic CD45 Fusion Proteins

CD45-CSR fusion proteins and/or CD45-ligand fusion proteins can be used to treat diseases that include inflammatory diseases, immune diseases, cancers, and other diseases that manifest aberrant angiogenesis or neovascularization or cell proliferation. Cancers include breast, lung, colon, gastric cancers, pancreatic cancers, and others. Inflammatory diseases include, for example, diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease, Alzheimer's disease and other neurodegenerative diseases, and other diseases known to those of skill in the art that involve proliferative response, immune responses and inflammatory responses and others in which CSRs are implicated, involved or in which they participate.

f. Methods for Measuring Glycosylation

Method for assessing the extent and pattern of glycosylation are provided herein. Modification of amino acid residues can be assessed by methods known by one of skill in art and can include techniques such as tryptic mapping, high liquid phase chromatography (HPLC), anion-exchange chromatography, circular dichromism, fluorophore labeling, mass spectrometry, crystallography, gel electrophoresis and enzymatic analysis of oligosaccharide release from PVDF membranes. Western blotting using panels of lectins that exhibit varying specificities and are conjugated with biotin or digoxigenin can identify a wide range of defined sugar epitopes found on glycoproteins. Western blotting also can be used to measure glycosylation of the CD45 extracellular domain specifically. Antibodies are available that detect extracellular domain of CD45 and can distinguish glycosylated variants of CD45.

g. Methods of Production and Increasing Glycosylation

Methods for the production of CD45 fusion proteins are provided herein. Mammalian expression systems as described in Section F3d can be used to express CD45 fusion proteins. Chinese hamster ovary (CHO) cell systems are often chosen for the production of glycoproteins since this cell type exhibits high expression of recombinant proteins and is capable of glycosylation of the proteins. Engineered human cell lines are also available, such as the GlycoExpress™ cell line (Glycotope), that are capable of producing glycoproteins with glycosylation patterns similar to endogenous wild-type human proteins. Engineered human cell lines are preferred for the production of therapeutic proteins as they possess the ability to properly sialylate glycosylated proteins, which affects the serum half-life and immunogenicity of a therapeutic glycoprotein.

h. HGF-CD45 Fusion Proteins and Therapeutic Uses

HGF isoforms can be fused to CD45, or a fragment thereof, to form a CD45-HGF fusion protein. HGF isoforms can be fused to a CD45 protein fragment, or combinations of fragments thereof, derived from the extracellular domain of CD45. Exemplary CD45 protein fragments include, but are not limited to, peptides set forth in SEQ ID NOS: 281, 283, 285, 287, 289, 291, 293, 295, and variants thereof. Exemplary CD45 protein fragments are encoded by nucleic acids set forth in SEQ ID NOS: 280, 282, 284, 286, 288, 290, 292, 294, and variants thereof. Exemplary HGF isoforms and allelic variants thereof that can be fused to a CD45 fragment include, but are not limited to, SEQ ID NOS: 10, 12 14, 18, and 20 Additional HGF polypeptides that can be fused to a CD45 fragment include, but are not limited to, polypeptides set forth in SEQ ID NOS: 3, 22, 24, 26, 28, 30, 32 and 246-251.

The CD45-HGF fusion protein can suppress, or alternatively, enhance HGF activity, such as angiogenesis, cell growth, morphogenesis, motogenesis, or tumor metastasis.

CD45-HGF fusion proteins can be used to treat, prevent, or ameliorate diseases that involve aberrant angiogenesis. CD45-HGF fusion proteins can be used to treat angiogeneic-related diseases, including but not limited to, rheumatoid arthritis, osteoarthritis, psoriasis, Osler-Webber syndrome, endometriosis, Still's disease, angiogenesis of the heart-muscle, peripheral hemangiectasis, hemophilic arthritis, age-related macular degeneration, retinopathy of prematurity, rejection to keratoplasty, systemic lupus erythematosus, atherosclerosis, neovascular glaucoma, choroidal neovascularization, retrolental fibroplasias, perosis, neurofibroma, hemangioma, acoustic neuroma, neurofibroma, trachoma, suppurative granuloma, and diabetes-related diseases, such as proliferative diabetic retinopathy and vascular diseases.

CD45-HGF fusion proteins can be used in the treatment and prevention of metastasis in cancers including, but not limited to, squamous cell cancer (e.g. epithelial squamous cell cancer), lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

### I. Methods of Preparing and Isolating HGF Isoform-Specific Antibodies

Methods of preparing and isolating antibodies, including polyclonal and monoclonal antibodies and fragments therefrom are well known in the art. Methods of preparing and isolating recombinant and synthetic antibodies also are well known in the art. For example, such antibodies can be constructed using solid phase peptide synthesis or can be produced recombinantly, using nucleotide and amino acid sequence information of the antigen binding sites of antibodies that specifically bind a candidate polypeptide. Antibodies also can be obtained by screening combinatorial libraries containing variable heavy chains and variable light chains, or antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an isoform polypeptide, for example, to detect the expression of an HGF isoform in a cell, tissue or extract.

### J. Assays to Assess or Monitor HGF Isoform Activities

Generally, the HGF isoforms provided herein exhibit an alteration in structure and also one or more activities compared to a wildtype or predominant form of a ligand. In particular, the isoforms exhibit HGF-antagonist activity and/or anti-angiogenic activity. As such the isoforms are candidate therapeutics. If needed, identified isoforms can be screened using in vitro and in vivo assays to monitor or identify an activity of an HGF isoform and to select HGF isoforms that exhibit such an activity or alteration in activity and/or that exhibit receptor binding or that modulate HGF-mediated MET activation and/or modulate growth factor angiogenic activity.

Any suitable assay can be employed, including assays exemplified herein. Numerous assays for activities of HGF are known to one of skill in the art. The assays permit comparison of an activity of an HGF isoform to an activity of a wildtype or predominant form of an HGF ligand to identify isoforms that lack an activity. In addition, assays permit identification of isoforms that modulate the activity of a MET receptor or other growth factor receptor such as those involved in angiogenesis including FGFR or VEGFR. Assays for HGF and HGF isoforms include, but are not limited to, ligand binding assays, receptor dimerization/oligomerization assays, MET and ERK phosphorylation assays, proliferation and mitogenic assays, motogenic assays, morphogenic assays, and apoptotic assays.

Alternatively or in addition, HGF isoforms modulate the activity of a MET and/or bind to or interact with other cell surface proteins such as GAGs, including heparin, or other cell surface proteins involved in angiogenesis, including growth factor receptors and other angiogenic inducing molecules such as αvβ3 integrin or angiomotin. Identified isoforms can be screened for such activities. Assays to screen isoforms to identify activities and functional interactions with MET and/or other cell surface proteins are known to those of skill in the art. One of skill in the art can test a particular isoform for interaction with MET or another cell surface protein and/or test to assess any change in activity compared to an HGF. Some are exemplified herein.

1. Ligand Binding Assays and HGF Binding Assays

HGF isoform binding can be assessed directly by assessing binding of an HGF isoform compared to HGF to cells. In some examples, binding of HGF isoforms to endothelial cells, or other cells known to bind HGF, can be assessed to determine generally if binding of an HGF isoform is altered compared to HGF; either enhanced or inhibited. In other examples, competitive assays can be employed with HGF or other known ligands for binding to cells known to express MET.

The ability of HGF isoforms to compete with HGF for binding to the MET receptor can be assessed. HGF and HGF isoforms are radioiodinated by the chloramine T method (see Nakamura et al., 1997, Cancer Res. 57, 3305-3313) and specific activities of ¹²⁵I-HGF and ¹²⁵I-HGF isoforms are measured. Cells that normally express the MET receptor are cultured in multiwell plates for the binding assay. The cells are equilibrated in an ice-cold binding buffer and incubated with various concentrations of ¹²⁵I-HGF or ¹²⁵I-HGF isoforms, with or without an excess molar ratio of unlabeled HGF or HGF isoforms. For competitive binding assays, a fixed concentration of ¹²⁵I-HGF and various concentrations of unlabelled HGF or HGF isoforms are incubated with the cells. After the incubation period, the cells are washed, solubilized, and the bound labeled proteins are measured using a γ-counter.

Binding of HGF to cell surface molecules, including MET or heparin, can be measured directly or indirectly for one or more than one cell surface molecule. For example, the ability of an HGF isoform to bind to heparin can be measured. In another assay, immunoprecipitation is used to assess cell surface molecule binding. Cell lysates are incubated with an HGF isoform. Antibodies against a cell surface molecule, such as αvβ3, heparin, or a growth factor receptor are used to immunoprecipitate the complex. The amount of HGF isoform in the complex is quantified and/or detected using western blotting of the immunoprecipitates with anti-HGF antibodies. Cell surface molecule binding assays also can include binding to ligands in the presence of other molecules. For example, cell surface molecule binding by HGF isoforms can be assessed in the presence of soluble heparin.

2. Ligand Dimerization

Dimerization of an HGF ligand, including an HGF isoform, can be tested to determine if the isoform forms dimers. For example, an isoform can be incubated in the presence or absence of a cross-linking reagent such as bis(sulfosuccinimidyl) suberate. In some examples, heparin can be added to the samples. Following quenching, the samples can be resolved by SDS-PAGE and protein can be detected by staining with Coomassie Blue protein stain or by using an anti-HGF antibody or anti-HGF isoform antibody. Protein bands can be analyzed to assess larger molecular weight bands compared to a protein not incubated with a cross-linking reagent, or a protein incubated in the absence of heparin, such as by assessing the presence of monomers, dimers, and other complexed forms within the samples.

3. Complexation

Complexation, such as dimerization of MET RTKs by an HGF ligand or HGF isoform can be detected and/or measured. Generally, receptor dimerization of an RTK is required for activation. An antagonist of MET signaling binds to MET but is unable to induce dimerization or activation. For example, isolated polypeptides can be mixed together, subject to gel electrophoresis and western blotting. HGF and/or HGF isoforms also can be added to cells and cell extracts, such as whole cell or fractionated extracts, and subjected to gel electrophoresis and western blotting. Antibodies recognizing the polypeptides can be used to detect the presence of monomers, dimers and other complexed forms. In some examples, heparin can be added, or cells can be treated with heparinase before complexation experiments.

4. MET and ERK1/2 Phosphorylation Assays

HGF isoforms can be assessed for their ability to affect activation of the MET receptor or interfere with HGF-induction of MET by measuring the phosphorylation status of MET. Endothelial cells that normally express the MET receptor, such as human dermal microvascular endothelial cells, are serum-starved overnight. The cells are then pre-treated with various concentrations of the HGF isoforms for 10 minutes followed by addition of either HGF or serum-free media. Cells can be treated with sodium orthovanadate (Na₃VO₄) alone for a positive control. After an incubation period, cells are washed and solubilized. Equivalent protein amounts of the cell extracts are immunoprecipitated with an anti-MET antibody, such as anti-MET C-12 (Santa Cruz Biotechnology, Santa Cruz, Calif.). The immunoprecipitates are washed, subjected to separation by SDS-PAGE, and transferred to a membrane. The amount of tyrosine phosphorylation of MET receptor is assessed by immunoreactivity with an anti-phosphotyrosine antibody, such as PY99 or PY20 (Santa Cruz Biotechnology, Santa Cruz, Calif. and Chemicon International, Inc., Temecula, Calif.).

Another indication of MET receptor induction is the activation of downstream kinases in the MET pathway. Kinases, such ERK1/2, are activated via phosphorylation following HGF-induced MET receptor activation. HGF isoforms can be assessed for their ability to affect ERK1/2 phosphorylation alone or in the presence of HGF. After treatment with HGF isoforms and/or HGF, as described above, whole cell extracts are subjected to SDS-PAGE and transferred to a membrane. The amount of phosphorylated ERK1/2 is assessed by immunoreactivity with an anti-phosphoERK1/2 antibody (New England Biolabs, Beverly, Mass.).

The assay for ERK1/2 phosphorylation can also be used to assess the ability of HGF isoforms to inhibit the activation of other angiogenic receptors, such as bFGF receptor and VEGF receptor. Following pretreatment with HGF isoforms, cells are incubated with bFGF or VEGF for a period of time. Whole cell extracts are assessed for phosphorylation of ERK1/2 as described above.

5. Morphogenic/Angiogenic Assays

The ability of HGF isoforms to affect HGF-induced angiogenesis in vitro can be assessed by measuring tubule formation. Endothelial cells, such as human umbilical vein endothelial cells (HUVECS) are plated into multiwell plates coated with Matrigel™ (BD Biosciences, San Jose, Calif.) and incubated overnight. The culture medium is then aspirated, and additional Matrigel™ containing either serum-free medium, HGF, HGF isoforms, or HGF with HGF isoforms in combination is overlaid onto the cells. After an overnight incubation, cells are observed under a phase contrast microscope. Random fields of cells are photographed and tubule length is measured. In place of HGF, HGF isoforms can also be co-incubated with other angiogenic factors that stimulate tubule formation, such as bFGF and VEGF, to assess the effects HGF isoforms have on the actions of other factors that stimulate angiogenesis.

Another version of this assay involves using HGF-producing fibroblasts, such as MRC5 cells, as the source of HGF. Endothelial cells, such as HUVECS, are plated into the lower chamber of a Transwell chamber (6.5 mm diameter polycarbonate membrane, 0.45 μm pore size, Costar) that has been coated with Matrigel™. After overnight incubation, the culture medium is then aspirated and additional Matrigel™ containing either serum-free medium or HGF isoforms is overlaid onto the cells. HGF-expressing MRC5 cells are plated in serum-free medium in the top chamber of the Transwell plate. Tubule length is measured after overnight incubation. Medium from the lower chamber is analyzed to confirm the presence of HGF by ELISA. RNA isolated from the MRC5 cells from the top chamber can also be analyzed by RT-PCR to assess production of HGF.

Three-dimensional culture in collagen gels can also be used to observe tubule formation in cells, such as MDCK cells. A defined amount of cells is suspended in 0.2% ice-cold collagen solution. After the solution is gelled, medium containing varying concentrations of HGF isoforms and/or HGF is added, and the cells are cultured for 6 hours. Control MDCK cells without HGF treatment will grow as spherical cysts, while treatment with HGF will induce branching tubulogenesis. Inhibition of tubule formation in the presence of HGF isoforms can be assessed by counting the number of tubules and the length of the tubules. In place of HGF, other angiogenic factors, such as FGF-2 and VEGF, can be used to stimulate tubulogenesis in the collagen gels, and the effects of HGF isoforms on their morphogenic activity can be assessed.

6. Mitogenic/Proliferation Assays

The effect of HGF isoforms on HGF-, FGF-2-, and VEGF-induced mitogenic activity of endothelial cells can be assessed by measuring cell proliferation. Endothelial cells at a predetermined density are plated onto gelatinized multiwell tissue culture plates and incubated overnight. Medium is replaced with fresh medium containing varying concentrations of HGF isoforms with HGF, FGF-2, VEGF or combinations thereof. After 72 hours, cells are dispersed with trypsin and counted using a Coulter counter. Quantitation of cell proliferation can also be performed using a 3-(4,5-dimethylthisazol-2-yl)-2,5-diphenyl tetrazolium bromide (MTT) method (see e.g., Yonekura et al. 2003 Biochem J. 370:1097-1109).

7. Motogenic/Cell Migration Assays

HGF isoforms can be assessed for their ability to interfere with HGF-induced cell motility. Endothelial cells are cultured in multiwell plates until firmly adhered to the culture dish surface. Fresh culture medium is then added and overlaid with light mineral oil to prevent evaporation. Medium containing HGF, HGF isoforms, or a combination thereof is added and images are recorded with a digital camera and a time lapse recorder. The distance traveled is calculated from a defined number of cells from each frame.

Effects of HGF isoforms on HGF-induced cell migration can also be assessed by an endothelial cell wounding assay. Endothelial cells are cultured on plates and grown to reach confluence. Cells are wounded with an 82-gauge needle to produce wounds of approximately 200 μm. The cells are then washed and fresh culture medium is added containing HGF isoforms, HGF, or a combination thereof. Images of cell migration are recorded as described above, and migration distance over the wound front is calculated.

Cell migration also can be assessed using a modified Boyden chamber assay. Endothelial cells, such as human dermal microvascular endothelial cells, are serum starved and then plated onto the inner chamber of a Transwell plate (6.5 mm diameter polycarbonate membrane, 5 μm pore size, Costar, Cambridge, Mass.) coated with 13.4 μg/ml fibronectin. Medium containing HGF, FGF-2 or VEGF, with or without HGF isoforms, is added to the outer chamber, and incubated for a period of time. The number of cells that migrate through the membrane to the under surface of the filter is quantified by counting the cells in randomly selected microscopic fields in each well.

Cell locomotion associated with dissociation of cells in response to HGF treatment can be analyzed by a cell scattering assay. The effects HGF isoforms have on HGF-induced cell scattering can be measured. Cells, such as MDCK renal epithelial cells, are cultured in multiwell plates in the presence of HGF isoforms, HGF, or a combination thereof. After overnight incubation, the cells are stained with hemotoxylin and photographed. Control cells, in the absence of HGF, will form tight colonies and maintain cell contacts, whereas HGF treatment induces scattering of the cells.

8. Apoptotic Assays

HGF exerts an anti-apoptotic effect on cells treated with cytotoxic agents, such as irradiation and certain cancer therapeutics, including cisplatin, camtothesin, Adriamycin, and taxol. The ability to HGF isoforms to alter the anti-apoptotic effects of HGF treatment can be measured. Cells are cultured with medium containing varying concentrations of HGF isoforms and/or HGF. Cells are then exposed to the cytotoxic agent for an incubation period, and cell viability is measured using a 3-(4,5-dimethylthisazol-2-yl)-2,5-diphenyl tetrazolium bromide (MTT, Sigma) assay.

Apoptotic cells show characteristic nuclear fragmentation that can be visualized by nuclear stains. Cells treated with HGF show reduced nuclear fragmentation in response to cytotoxic agents. The ability of HGF isoforms to antagonize this effect of HGF can be assessed. Cells are plated onto glass slides and treated with cytotoxic agents followed by HGF and/or HGF isoforms as described above. Nuclei of the cells are visualized using Hoescht 33342 stain and a fluorescent microscope at excitement wavelength of 350 nm and emission wavelength of 450 nm.

Nuclear fragmentation is the result of cleavage of genomic DNA during apoptosis and yields double stranded and single strand breaks (“nicks”) that produce laddering of chromosomal DNA, which is inhibited by treatment with HGF. A DNA fragmentation assay can be used to measure the degree of chromosomal DNA laddering in response to cytotoxic agents in the presence of HGF isoforms and/or HGF. After treatment, cells are solubilized, and RNA and proteins are removed from the sample by treatment with RNase A and proteinase K, respectively. Chromosomal DNA is precipitated and electrophoresed on an agarose gel with ethidium bromide to visualize DNA ladders present in apoptotic cells.

The DNA filter elution assay also can be used to measure the degree of DNA breakage in apoptotic cells. Cells are incubated with [³H] thymidine for 32 hours followed by incubation in isotope free medium for 2 hours. The cells are then pretreated with varying concentrations of the HGF isoforms and/or HGF, followed by treatment with a cytotoxic agent. After an overnight incubation, the cells are resuspended in trypsin and applied to polycarbonate membranes. The cells are lysed on the membrane and alkaline eluted for detection of single strand DNA breaks. The samples are counted on a scintillation counter and measured as Dpm eluted/(dpm filter-bound+dpm total lysates). Larger unfragmented DNA pieces elute more slowly; hence, the amount of DNA eluted as a function of time is proportional to the DNA damage.

Another method of measuring DNA breakage is the TUNEL stain, which identifies DNA breaks by labeling free 3′-OH termini with modified nucleotides in an enzymatic reaction. This protocol can detect and quantify apoptosis at the single cell level. Commercial kits are available for TUNEL staining, including Apotag In situ Apoptosis detection kit (Invitrogen, Carlsbad, Calif.). Following treatment with HGF isoforms and/or HGF as described above, cells are trypsinized and transferred to glass slides by cytospin centrifugation. Cells are permeabilized followed by immunocytochemistry, which involves the addition of terminal deoxynucleotidyl transferase, digoxygenin-dUTP, anti-digoxygenin HRP, and diaminobenzidine. The cells are counterstained with methyl green and quantified by counting the number of TUNEL-positive cells among a predefined number of cells per slide. The fraction of cells labeled is expressed as an apoptotic index. A positive control for this experiment can include incubation of cells with DNase I to induce DNA strand breaks prior to the labeling procedure.

Measurement of caspase-3 activity can be another measure of induction of the apoptotic program. After treatment with HGF isoforms and/or HGF as described above, cells are solubilized and incubated with the fluorogenic substrate Ac-DEVD-AFC. An inhibitor for caspase-3, Z-DEVD-CMK (Bio-Rad), can be used for a control. Cleavage of the substrate is assessed by a spectrofluorimeter at excitation wavelength 400 nm and emission wavelength 520 nm. Activity is determined by subtracting the peak values in the presence of the control inhibitor.

The anti-apoptotic effects of HGF are believed to be mediated via activation of AKT through the phophatidylinositol-3-kinase (PI3K) pathway. In vitro kinase assays can be performed to assess the ability of HGF isoforms to inhibit HGF induction of AKT activity. SK-LMS-1 cells, which overexpress MET receptor, are transfected with plasmids encoding HA-AKT1 or HA-AKT2, and serum starved overnight. Cells are then treated with varying concentrations of HGF isoforms and/or HGF. Treatment with PI3K inhibitors, such as wortmannin or LY294002, prior to addition of HGF can be used as a positive control for AKT inhibition. Cells are lysed and AKT protein is immunoprecipitated using anti-HA antibodies and/or anti-AKT antibodies. Half of the sample is used for normalizing AKT protein amount. The other half of the sample is used for a kinase assay, in which AKT protein is incubated with [γ-³²P]ATP and histone 2B as a substrate. Samples are subjected to SDS/PAGE and autoradiography.

9. Animal Models

a. Tumor Suppression Assays

Numerous assays are known to those of skill in the art to assess the effects of HGF isoforms on tumor growth and metastasis. Models for various cancers affected by the HGF-MET pathway can include injection of cells or cell lines, including cancerous cells or cells transformed with various growth factors, into target tissues. For example, subcutaneous injection of athymic nude mice with C-127 cells transformed with human HGF and mouse MET produces metastatic tumors in the mice within 2-3 weeks. Recombinant HGF isoforms can be injected at regular intervals for a period of time and tumor size can be measured. In addition, combination therapies including radiation or chemotherapeutic drugs can be delivered in addition to HGF isoform treatment to examine additive or synergistic effects of the anti-tumor therapy.

Some examples of animal models of cancer that are useful for testing HGF isoform treatment in specific cancers can include:

Glioma. Malignant gliomas are the most common cancer of the central nervous system and are associated with poor prognosis due to innate resistance to radio- and chemo-therapy. Malignant gliomas express high levels of HGF. Inhibiting HGF signaling has been shown to reverse malignant phenotypes in vitro and in vivo. Mouse models of gliomas involve injection of 9L cells transformed with HGF injected into caudate-putamen of rats to produce brain tumors. Injections of HGF isoforms can be done to analyze size and metastasis of these tumors.

Other models of glioma include xenografts of cell lines, such as the U-118 human glioma cell line (GBM) that is autocrine for endogenous HGF/MET signaling. GBM cells can be injected subcutaneously into athymic nude mice to produce tumors, and the effects of HGF isoforms on tumor growth can be assessed. Injections of HGF isoforms can be done starting at the time of the xenograft injection or, alternatively, HGF isoforms can be injected intratumor once the tumor has been established.

Colon cancer. Colon cancer is one of the most common cancers in humans. It is characterized by a high mortality rate due to metastatic disease caused by a high rate of metastasis in the liver. Mouse models of colon cancer metastasis involve injection of MC-38 colon cancer cells into spleens of mice. Metastatic nodules are observed at about 21 days after inoculation. Following treatment with HGF isoforms, the number and size of the tumor nodules, blood vessel density in the nodules, number of apoptotic cells in the nodules, and the degree of MET activation can be assessed.

Pancreatic Cancer. Pancreatic cancer is a highly malignant form of cancer with a severely poor prognosis. A mouse model of pancreatic cancer involves orthotopic injection of SUIT-2 cells, a pancreatic cancer cell line, into the pancreas of nude mice. Within 4 weeks the mice will develop a large mass that disseminates into the peritoneal cavity. Treatment with HGF isoforms can be done starting at various times during tumor growth to assess survival and tumor growth.

Additional mouse models for the study of HGF in cancer progression that are available to those of skill in the art can include, but are not limited to: gastric carcinoma, gall bladder carcinoma, lung carcinoma, lymphoma, hepatocellular carcinoma, malignant melanoma, mammary carcinoma, and ovarian carcinoma.

b. Angiogenic Disease

Animals models for diseases associated with excessive neovascularization are known to those of skill in the art. In addition to assays of tumor angiogenesis in animal cancer models, animal models are available for the study of diseases such as proliferative diabetic retinopathy. One such model involves transgenic mice that overexpress insulin-like growth factor (IGF-1) in the eye. These mice exhibit vascular occlusion of retinal vessels, venous dilatation and beading, widespread capillary non-perfusion areas, intraretinal microvascular abnormalities (IRMA) and neovascularization within the retina and inside the vitreous. Treatment with HGF isoforms can be done to observe effects of inhibition of HGF signaling on vessel formation. Also the effects HGF isoforms in the presence of angiogenic factors such as VEGF and/or FGF-2 can be studied.

An additional model for studying vessel proliferation is the corneal micropocket assay, in which corneal neovascularization is induced by pellets containing FGF-2 or VEGF implanted into the corneas of rabbits. The degree of neovascularization in the cornea can be measured in terms of vessel length, number, and branching. Effects of HGF isoforms can be assessed by implantation, injection, or other delivery methods known in the art.

### K. Preparation, Formulation and Administration of HGF Isoforms and HGF Isoform Compositions

HGF isoforms and HGF isoform compositions can be formulated for administration by any route known to those of skill in the art including intramuscular, intravenous, intradermal, intraperitoneal, subcutaneous, epidural, nasal, oral, rectal, topical, inhalational, buccal (e.g., sublingual), and transdermal administration or any route. HGF isoforms can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case depends upon a variety of factors, such as the nature of the disease, the progress of the disease, the severity of the disease and the particular composition that is used.

Various delivery systems are known and can be used to administer HGF isoforms, such as, but not limited to, encapsulation in liposomes, microparticles, microcapsules, recombinant cells capable of expressing the compound, receptor mediated endocytosis, and delivery of nucleic acid molecules encoding HGF isoforms such as retrovirus delivery systems.

Pharmaceutical compositions containing HGF isoforms can be prepared. Generally, pharmaceutically acceptable compositions are prepared in view of approval by a regulatory agency or otherwise prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non-toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly(alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of HGF isoforms can be formulated for parenteral administration by injection e.g., by bolus injection or continuous infusion. Formulations for injection can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles also can be employed with HGF isoform administration. Liposomes are formed from phospholipids that are dispersed in an aqueous medium and spontaneously form multilamellar concentric bilayer vesicles, also termed multilamellar vesicles (MLVs). MLVs generally have diameters of from 25 nm to 4 μm. Sonication of MLVs results in the formation of small unilamellar vesicles (SUVs) with diameters in the range of 200 to 500. ANG., containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

Administration methods can be employed to decrease the exposure of HGF isoforms to degradative processes, such as proteolytic degradation and immunological intervention via antigenic and immunogenic responses. Examples of such methods include local administration at the site of treatment. Pegylation of therapeutics has been reported to increase resistance to proteolysis, increase plasma half-life, and decrease antigenicity and immunogenicity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10): 1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how, and when, to adjust treatment to higher levels if the clinical response is not adequate (precluding toxic side effects). The active agent is administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

An HGF isoform is included in the pharmaceutically acceptable carrier in an amount sufficient to exert a therapeutically useful effect in the absence of undesirable side effects on the patient treated. Therapeutically effective concentrations can be determined empirically by testing the compounds in known in vitro and in vivo systems, such as the assays provided herein.

The concentration of an HGF isoform in the composition depends on absorption, inactivation and excretion rates of the complex, the physicochemical characteristics of the complex, the dosage schedule, and amount administered as well as other factors known to those of skill in the art. The amount of an HGF isoform to be administered for the treatment of a disease or condition, for example cancer or angiogenesis treatment, can be determined by standard clinical techniques. In addition, in vitro assays and animal models can be employed to help identify optimal dosage ranges. The precise dosage, which can be determined empirically, can depend upon the route of administration and the seriousness of the disease. Suitable dosage ranges for administration can range from about 0.01 pg/kg body weight to 1 mg/kg body weight and more typically 0.05 mg/kg to 200 mg/kg HGF isoform: patient weight.

An HGF isoform can be administered once, or can be divided into a number of smaller doses to be administered at intervals of time. HGF isoforms can be administered in one or more doses over the course of a treatment time, for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. The mode of administration of the composition containing the polypeptides as well as compositions containing nucleic acids for gene therapy, includes, but is not limited to intralesional, intraperitoneal, intramuscular and intravenous administration. Also included are infusion, intrathecal, subcutaneous, liposome-mediated and depot-mediated administration. Also included are nasal, ocular, oral, topical, local and otic delivery. Dosages can be empirically determined and depend upon the indication, mode of administration and the subject. Exemplary dosages include from 0.1, 1, 10, 100, 200 and more mg/day/kg weight of the subject.

### L. In Vivo Expression of HGF isoforms and Gene Therapy

HGF isoforms can be delivered to cells and tissues by expression of nucleic acid molecules. HGF isoforms can be administered as nucleic acid molecules encoding an HGF isoform, including ex vivo techniques and direct in vivo expression.

1. Delivery of HGF

Nucleic acids can be delivered to cells and tissues by any method known to those of skill in the art.

a. Vectors—Episomal and Integrating

Methods for administering HGF isoforms by expression of encoding nucleic acid molecules include administration of recombinant vectors. The vector can be designed to remain episomal, such as by inclusion of an origin of replication or can be designed to integrate into a chromosome in the cell. Recombinant vectors can include viral vectors and non-viral vectors. Non-limiting viral vectors include, for example, adenoviral vectors, herpes virus vectors, retroviral vectors, and any other viral vector known to one of skill in the art. Non-limiting non-viral vectors include artificial chromosomes or liposomes or other non-viral vectors. HGF isoforms also can be used in ex vivo gene expression therapy using viral and non-viral vectors. For example, cells can be engineered to express an HGF isoform, such as by integrating an HGF isoform-encoding nucleic acid into a genomic location, either operatively linked to regulatory sequences or such that it is placed operatively linked to regulatory sequences in a genomic location. Such cells then can be administered locally or systemically to a subject, such as a patient in need of treatment.

An HGF isoform can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with an HGF isoform-expressing adenovirus vector. After a suitable culturing period, the transduced cells are administered to a subject, locally and/or systemically. Alternatively, HGF isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell.

b. Artificial Chromosomes and Other Non-Viral Vector Delivery Methods

The nucleic acid molecules can be introduced into artificial chromosomes and other non-viral vectors. Artificial chromosomes (see, e.g., U.S. Pat. No. 6,077,697 and PCT International PCT application No. WO 02/097059) can be engineered to encode and express the isoform.

c. Liposomes and Other Encapsulated Forms and Administration of Cells Containing the Nucleic Acids

The nucleic acids can be encapsulated in a vehicle, such as a liposome, or introduced into a cell, such as a bacterial cell, particularly an attenuated bacterium, or introduced into a viral vector. For example, when liposomes are employed, proteins that bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

2. In Vitro and Ex Vivo Delivery

For ex vivo and in vivo methods, nucleic acid molecules encoding the HGF isoform is introduced into cells that are from a suitable donor or the subject to be treated. Cells into which a nucleic acid can be introduced for purposes of therapy include, for example, any desired, available cell type appropriate for the disease or condition to be treated, including but not limited to, epithelial cells, endothelial cells, keratinocytes, fibroblasts, muscle cells, hepatocytes, blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For ex vivo treatment, cells from a donor compatible with the subject to be treated, or cells from the subject to be treated, are removed, the nucleic acid is introduced into these isolated cells and the modified cells are administered to the subject. Treatment includes direct administration, such as, for example, encapsulated within porous membranes, which are implanted into the patient (see, e.g. U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol), electroporation, microinjection, cell fusion, DEAE-dextran, and calcium phosphate precipitation methods. Methods of DNA delivery can be used to express HGF isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of an HGF isoform can be linked to expression of additional molecules. For example, expression of an HGF isoform can be linked with expression of a cytotoxic product such as in an engineered virus or expressed in a cytotoxic virus. Such viruses can be targeted to a particular cell type that is a target for a therapeutic effect. The expressed HGF isoform can be used to enhance the cytotoxicity of the virus.

In vivo expression of an HGF isoform can include operatively linking an HGF isoform encoding nucleic acid molecule to specific regulatory sequences such as a cell-specific or tissue-specific promoter. HGF isoforms also can be expressed from vectors that specifically infect and/or replicate in target cell types and/or tissues. Inducible promoters can be use to selectively regulate HGF isoform expression.

3. Systemic, Local and Topical Delivery

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating cells can be targeted cells for in vivo expression of HGF isoforms. Cells used for in vivo expression of an isoform also include cells autologous to the patient. Such cells can be removed from a patient, nucleic acids for expression of an HGF isoform introduced, and then administered to a patient such as by injection or engraftment.

### M. HGF and Cancer and Angiogenesis

HGF plays a significant role in mediating mitogenesis, morphogenesis, motogenesis, and angiogenesis through its receptor MET. In cancer, these activities are involved in the growth, neovascularization, and metastasis of tumors (see e.g., FIG. 1). Metastases of primary tumors are often associated with high mortality rates in cancer patients, and treatments that decrease the metastatic processes of tumor growth, including tumor-induced angiogenesis, may elevate the prognoses in malignant cancers. In addition to cancer, the angiogenic properties of HGF contribute to the progression of various vascular diseases, including rheumatoid arthritis and proliferative diabetic retinopathy. HGF isoforms, such as HGF isoforms provided herein, can be used as antagonists of MET to inhibit cancer growth and spread and also can be used as general angio-inhibitory molecules to inhibit angiogenesis associated with cancer progression or other vascular diseases.

1. Tumor Growth and Metastasis

HGF regulates cellular processes including proliferation, apoptosis, migration, and morphogenesis, which contribute to the invasive, angiogenic, and metastatic responses associated with malignant behavior in cancer. The receptor for HGF, MET, was originally isolated as an oncogenic fusion protein, encoded by tpr-met, with constitutive, ligand-independent tyrosine kinase activity and the ability to transform cells. Excessive activation of MET can induce tumor growth, tumor cell motility, invasion of extracellular matrices and angiogenesis. A large number of cancers, including carcinomas of the bladder, breast, cervix, colon, esophagus, stomach, head and neck, kidney, liver, lung, pharynx, ovary, pancreas, prostate and thyroid, musculoskeletal sarcomas, soft tissue sarcomas, hematopoietic malignancies, glioblastomas, melanomas, mesotheliomas and Wilm's Tumor, exhibit elevated levels of HGF and/or MET expression that contribute to autocrine upregulation of HGF signaling. Mutations in the c-met gene also have been identified in carcinomas of the stomach, head and neck, kidney, liver, lung, ovary, and thyroid. Transgenic mice that are engineered to express high levels HGF develop a broad array of histologically distinct tumors of mesenchymal and epithelial origin. In animal models of cancers with elevated MET and/or HGF expression, treatments with inhibitors that block activation of the MET receptor have been successful in affecting tumor growth and metastasis.

Progression of cancer from transformation to malignancy and metastasis is a multistep process that involves enhanced cellular proliferation, evasion of cell death, disruption of cell-cell contacts, degradation of the extracellular matrix, and increased cell motility and morphogenesis. HGF has been implicated in the regulation of each of these processes relating to the establishment and invasiveness of the primary tumor and to the metastatic cascade, whereby cells detach from the primary tumor and travel via the circulatory system to distal sites to form secondary tumors.

a. Mitogenesis

Stimulation of HGF can induce cellular proliferation. Although the mitogenic potential of HGF can vary depending on the type of cancer, HGF clearly exhibits several cell cycle promoting activities. HGF treatment can induce mitogenic signaling pathways such as the MEK/ERK pathway. HGF can also down-regulate p27kip1, which causes an accumulation of hyperphosphorylated Rb protein that advances cell cycle entry. In addition, HGF signaling can lead to the accumulation of β-catenin, which promotes formation of the LEF/TCF transcription factor complex that upregulates cell cycle regulators involved in oncogenic transformation.

The mitogenic properties of HGF are also linked to inhibition of apoptosis. Upregulation of cellular survival factors is a critical feature of cancer cells and contributes to their ability to escape apoptotic cell death. HGF treatment has been shown to protect cells against apoptosis induced by serum starvation, UV irradiation, and other cytotoxic agents. Constitutive expression of activated MET in cells, such as hepatocytes, can also inhibit apoptosis. The anti-apoptotic effects of HGF are mediated in part by activation of Akt kinase via the phosphatidylinositol 3-kinase (PI3K) pathway. In support of this, studies have shown that the anti-apoptotic effects of HGF can be blocked by treatment with PI3K inhibitors, such as LY294002. In addition, HGF can induce the expression and/or activation of anti-apoptotic proteins, including BCL-xL, MAPK, and GATA-4. HGF signaling can also interfere with the activation of certain caspases that are important for the apoptotic program. The MET receptor also has the ability to directly bind to Fas and prevent Fas-induced apoptosis.

HGF treatment can inhibit the apoptotic effects induced by DNA damaging agents, including cytotoxic agents used in the treatment of cancer. Studies have shown that HGF treatment promotes cell survival in lung cancer, glioblastoma cells, colon cancer cells, breast cancer cells, squamous cell carcinoma of the head and neck, myeloma cells, and in epithelial cell lines. As an example, HGF treatment of MDA-MB-453 human breast cancer cells, EMT6 mouse mammary tumor cells, U373 glioblastoma cells or MDCK renal epithelial cells protects the cells against apoptosis induced by cytotoxic agents, such as adriamycin (ADR), cisplatin, camtothesin, taxol, X-rays, gamma irradiation, or ultraviolet radiation. Given the effects of HGF on the inhibition of apoptosis, accumulation of HGF in cancerous cells may contribute to radio- and chemo-resistant phenotypes that have been observed in cancer therapy. Inhibitors of HGF signaling thus have the potential to be used in combination therapies with conventional cytotoxic agents for the treatment of cancer.

b. Motogenesis and Morphogenesis

The ability of cancer cells to invade surrounding tissue and to migrate to distal sites depends on the stimulation of cell motility and involves the morphogenesis of epithelial and endothelial cell types. These processes are also important for normal organ development and wound healing. In cancer, however, dysregulation of cell motility and morphogenesis contributes to cancer progression and metastasis. It is also important for tumor angiogenesis as discussed below. Treatment of cancer cells with HGF promotes rapid migration of cells over a number of matrices. HGF can stimulate movement and morphogenesis of cells though activation of components of the rho/rac pathway. This pathway is important for cytoskeletal rearrangement and cell-substrate adhesion. Several members of the rho family are aberrantly expressed in cancers. Upon HGF stimulation, the MET receptor is phosphorylated on multiple tyrosine residues that serve as docking sites for signaling molecules, including c-Cbl, PI3K, Grb2, Shc, Crk, and Gab-1. These proteins in turn activate downstream signaling pathways that connect with the cytoskeletal machinery leading to breakdown of adherens junctions, stimulation of membrane ruffling, and directional cell movement.

Another important factor in cell migration in tumor metastasis is the disruption of cell contacts to promote dissociation and scattering of cells from their anchored positions. HGF signaling promotes cell scattering via β-catenin assisted pathways leading to shedding and redistribution of cadherins, such as E-cadherin, that are important for maintaining cell-cell contacts.

Invasion of cancer cells into the surrounding tissue also requires the degradation of the extracellular matrix. HGF contributes to the invasiveness of cancer cells through stimulation of proteolytic enzyme secretion, including matrix metalloproteinases, such as MMp2, MMP7, and MMp9, and serine proteases such as the plasminogen activator uPA. This breakdown of the extracellular matrix aids in migration of the metastatic cells from the primary tumor site and in the invasive ability of the cells at distal docking sites. HGF is also often stored within the extracellular matrix in the tumor tissue. Following secretion of proteolytic enzymes, this ready source of HGF is released, further aiding in the cancer cell migration and invasion. Heparin sulfate glycosaminoglycans in the extracellular matrix can also bind to MET independently of HGF and may regulate motility.

At distal locations of secondary tumor growth, cell surface molecules such as CD44 and integrins play a role in anchoring the metastatic cell to the distal site of invasion. HGF expression can induce the expression of CD44. In addition, MET plays a critical role in docking via its interaction with integrins, such as α6β4 integrin.

2. Angiogenesis

Cellular receptors for angiogenic factors (positive and negative) can act as points of intervention in multiple disease processes, for example, in diseases and conditions where the balance of angiogenic growth factors has been altered and/or the amount or timing of angiogenesis is altered. For example, in some situations ‘too much’ angiogenesis can be detrimental, such as angiogenesis that supplies blood to tumor foci, and in inflammatory responses and other aberrant angiogenic-related conditions. The growth of tumors, or sites of proliferation in chronic inflammation, generally requires the recruitment of neighboring blood vessels and vascular endothelial cells to support their metabolic requirements. This is because the diffusion is limited for oxygen in tissues. Exemplary conditions that require angiogenesis include, but are not limited to solid tumors and hematologic malignancies such as lymphomas, acute leukemia and multiple myeloma, where increased numbers of blood vessels are observed in the pathologic bone marrow. Stimuli for angiogenesis include hypoxia, inflammation and genetic lesions in oncogenes or tumor suppressors that alter disease cell gene expression.

a. The Angiogenic Process

Angiogenesis includes several steps, including the recruitment of circulating endothelial cell precursors (CEPs), stimulation of new endothelial cell (EC) growth by growth factors, the degradation of the ECM by proteases, proliferation of ECs and migration into the target, which could be a tumor site or another proliferative site caused by inflammation. This results in the eventual formation of new capillary tubes. Such blood vessels are not necessarily normal in structure. They may have chaotic architecture and blood flow. Due to an imbalance of angiogenic regulators such as vascular endothelial growth factor (VEGF), and angiopoietins, the new vessels supplying tumorous or inflammatory sites are tortuous and dilated with an uneven diameter, excessive branching, and shunting. Blood flow is variable, with areas of hypoxia and acidosis leading to the selection of variants that are resistant to hypoxia-induced apoptosis (often due to the loss of p53 expression); and enhanced production of pro-angiogenic signals. Disease-associated vessel walls have numerous openings, widened interendothelial junctions, and a discontinuous or absent basement membrane; this contributes to the high vascular permeability of these vessels and, together with lack of functional lymphatics/drainage, causes interstitial hypertension. Disease-associated blood vessels may lack perivascular cells such as pericytes and smooth muscle cells that normally regulate vasoactive control in response to tissue metabolic needs. Unlike normal blood vessels, the vascular lining of tumor vessels is not a homogenous layer of ECs but often contains a mosaic of ECs and tumor cells; the concept of cancer cell-derived vascular channels, which may be lined by ECM secreted by the tumor cells, is referred to as vascular mimicry.

A similar situation occurs where blood vessels rapidly invade sites of acute inflammation. The ECs of angiogenic blood vessels are unlike quiescent ECs found in adult vessels, where only 0.01% of ECs are dividing. During tumor angiogenesis, ECs are highly proliferative and express a number of plasma membrane proteins that are characteristic of activated endothelium, including growth factor receptors and adhesion molecules such as integrins. Tumors utilize a number of mechanisms to promote their vascularization, and in each case they subvert normal angiogenic processes to suit this purpose. For this reason, increased production of angiogenic factors, proliferative with respect to endothelium and structure (allowing for increased branching of the neovasculature), are likely to occur in disease foci, as in cancer or chronic inflammatory disease.

b. Cell Surface Receptors in Angiogenesis

Cell surface receptors, including receptor tyrosine kinases (RTKs) and their ligands, play a role in the regulation of angiogenesis. Angiogenic endothelium expresses a number of receptors not found on resting endothelium. These include RTKs (i.e. FGF, PDGF and VEGF receptors) and integrins that bind to the extracellular matrix and mediate endothelial cell adhesion, migration, and invasion. Functions mediated by activated RTK include proliferation, migration, and enhanced survival of endothelial cells, as well as regulation of the recruitment of perivascular cells and bloodborne circulating endothelial precursors and hematopoietic stem cells to the tumor.

Additional signaling pathways also are involved in angiogenesis. The angiopoietin, Ang1, produced by stromal cells, binds to the RTK Tie-2 and promotes the interaction of endothelial cells with the extracellular matrix and perivascular cells, such as pericytes and smooth muscle cells, to form tight, non-leaky vessels. PDGF and basic fibroblast growth factor (bFGF, also called FGF-2) help to recruit these perivascular cells. Ang1 is required for maintaining the quiescence and stability of mature blood vessels and prevents the vascular permeability normally induced by VEGF and inflammatory cytokines.

Pro-angiogenic cytokines, chemokines, and growth factors secreted by stromal cells or inflammatory cells make important contributions to neovascularization, including bFGF, transforming growth factor-alpha, TNF-alpha, and IL-8. In contrast to normal endothelium, angiogenic endothelium overexpresses specific members of the integrin family of extracellular matrix-binding proteins that mediate endothelial cell adhesion, migration, and survival. Integrins mediate spreading and migration of endothelial cells and are required for angiogenesis induced by HGF, VEGF and bFGF, which in turn can upregulate endothelial cell integrin expression. VEGF promotes the mobilization and recruitment of circulating endothelial cell precursors (CEPs) and hematopoietic stem cells (HSCs) to tumors where they colocalize and appear to cooperate in neovessel formation. CEPs express VEGFR2, while HSCs express VEGFR1, a receptor, or VEGF and PlGF. Both CEPs and HSCs are derived from a common precursor, the hemangioblast. CEPs are thought to differentiate into endothelial cells, whereas the role of HSC-derived cells (such as tumor-associated macrophages) may be to secrete angiogenic factors required for sprouting and stabilization of endothelial cells (VEGF, bFGF, angiopoietins) and to activate matrix metalloproteinases (MMPs), resulting in extracellular matrix remodeling and growth factor release. In mouse tumor models and in human cancers, increased numbers of CEPs and subsets of VEGFR1 or VEGFR-expressing HSCs can be detected in the circulation, which may correlate with increased levels of serum VEGF. HGF also contributes to normal physiological angiogenesis that occurs during embryonic development, wound healing, and tissue regeneration.

c. HGF in Tumor Angiogenesis

Neovascularization is a critical process in tumor growth. A critical element in the growth of primary tumors and formation of metastatic sites is the ability of the tumor to promote the formation of new capillaries from preexisting host vessels. Tumor-associated angiogenesis is a complex process involving many different cell types that proliferate, migrate, invade, and differentiate in response to signals from the microenvironment. Endothelial cells sprout from host vessels in response to HGF, VEGF, bFGF, Ang2, and other pro-angiogenic stimuli. Sprouting is stimulated by HGF/MET, VEGF/VEGFR2, Ang2/Tie-2, and integrin/extracellular matrix interactions. Bone marrow-derived circulating endothelial precursors migrate to the tumor in response to VEGF and differentiate into endothelial cells, while hematopoietic stem cells differentiate into leukocytes, including tumor-associated macrophages that secrete angiogenic growth factors and produce matrix metalloproteinases (MMPs) that remodel the extracellular matrix and release bound growth factors.

HGF contributes to angiogenesis by stimulation of morphogenic changes that promote angiogenesis in vascular endothelial cells. HGF signaling stimulates branching tubulogenesis in endothelial cells and alters endothelial cell motility. HGF can also upregulate the expression of angiogenic factors, including VEGF and IL-8, and the downregulation angiogenic suppressive factors, such as thrombospondin-1 (TPS-1), which inhibit endothelial cell proliferation and induce endothelial cell apoptosis. HGF also takes part in mediating epithelial to mesenchymal transition and formation of tubule and lumens necessary for angiogenesis. Although HGF signaling through the MET receptor plays an import role in the morphological changes associated with angiogenesis, studies with HGF antagonists have revealed that HGF angiogenic activities may partially function though activation of FGF and/or VEGF receptors.

When tumor cells arise in, or metastasize to, an avascular area, they grow to a size limited by hypoxia and nutrient deprivation. This condition, also likely to occur in other localized proliferative diseases, leads to the selection of cells that produce angiogenic factors. Hypoxia, a key regulator of tumor angiogenesis, causes the transcriptional induction of VEGF and HGF by a process that involves stabilization of the transcription factor hypoxia-inducible factor (HIF)1. Under normoxic conditions, EC HIF-1 levels are maintained at a low level by proteasome-mediated destruction regulated by a ubiquitin E3-ligase encoded by the VHL tumor-suppressor locus. Under hypoxic conditions, the HIF-1 protein is not hydroxylated and association with VHL does not occur; therefore HIF-1 levels increase, and target genes including HGF, VEGF, nitric oxide synthetase (NOS), and Ang2 are induced. Loss of the VHL genes, as occurs in familial and sporadic renal cell carcinomas, also results in HIF-1 stabilization and induction of VEGF. Most tumors have hypoxic regions due to poor blood flow, and tumor cells in these areas stain positive for HIF-1 expression.

d. HGF in Other Vascular Diseases

Angiogenesis also plays a role in inflammatory diseases. These diseases have a proliferative component, similar to a tumor focus. In rheumatoid arthritis, one component of this is characterized by aberrant proliferation of synovial fibroblasts, resulting in pannus formation. The pannus is composed of synovial fibroblasts which have some phenotypic characteristics with transformed cells. As a pannus grows within the joint it expresses many pro-angiogenic signals, and experiences many of the same neo-angiogenic requirements as a tumor. The need for additional blood supply, neoangiogenesis, is critical. Similarly, many chronic inflammatory conditions also have a proliferative component in which some of the cells composing it may have characteristics usually attributed to transformed cells.

Another example of a condition involving excess angiogenesis is proliferative diabetic retinopathy (PDR) (Lip et al. Br J Ophthalmology 88: 1543, 2004). PDR possesses angiogenic, inflammatory and proliferative components. It is characterized by neovascularization of the retina and intrusion of vessels into the vitreous cavity, and is accompanied by bleeding and scarring around proliferative channels. Elevated expression of HGF, VEGF, and angiopoietin-2 is commonly detected in the vitreous fluid of patients with PDR. This overexpression is likely required for disease-associated remodeling and branching of blood vessels, which then supports the proliferative component of the disease. VEGF may be important in early stage to increase vascular permeability while HGF functions at a later stage in growth of endothelial cells in neovascularization.

3. HGF Isoforms and Cancer and Angiogenesis

HGF isoforms that antagonize HGF/MET signaling and/or that inhibit angiogenesis can be used in treatments of cancer and angiogenic related vascular disease. Generally, angiogenesis inhibitors are potent inhibitors of tumor growth and metastases by decreasing the density of blood vessels that supply oxygen to the tumor. Metastasis of tumors, however, is also contributed to by hypoxic regions of tumors that are devoid of vessel growth. Such hypoxia leads to upregulation of MET in cancer cells which in turn leads to invasive growth potential of tumors through upregulation of the HGF/MET signaling pathway. Thus, inhibition of HGF/MET signaling offers added advantages of decreasing metastatic growth coupled with anti-angiogenic therapy.

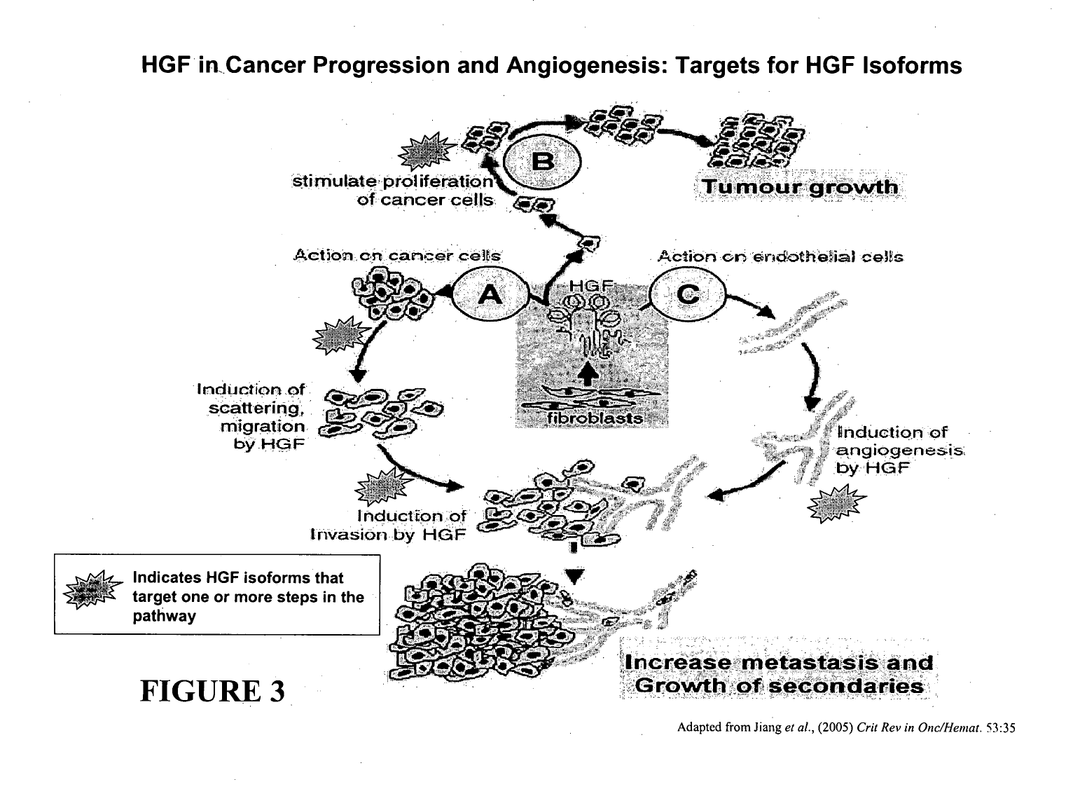

Provided herein are HGF isoforms that can modulate one or more steps in the tumorogenic and/or angiogenic process. Exemplary steps in the tumor growth and angiogenesis pathway that are targets for HGF isoforms are shown in FIG. 3. HGF isoforms can be administered singly, intermittently, together in single or two or more compositions or in other combinations thereof. Among the isoforms provided are those that compete with HGF for binding to MET and/or other receptors therefore thereby reducing interaction of circulating HGF. Reduction of circulating HGF can mitigate the effects of circulating HGF in cancer development, including inhibiting tumor growth, invasion, and metastasis of tumor cells and its role in angiogenesis. HGF isoforms also can inhibit angiogenesis as it contributes to the metastasis and growth of primary and secondary tumors, as well as other angiogenic diseases.

### N. Exemplary Treatments with HGF Isoforms

Provided herein are methods of treatment with HGF isoforms for diseases and conditions associated with angiogenesis and/or aberrant activation of MET. HGF isoforms can be used in the treatment of a variety of diseases and conditions, including those described herein. Treatment can be effected by administering, by suitable route, formulations of the polypeptides, which can be provided in compositions as polypeptides and can be linked to targeting agents for targeted delivery, or encapsulated in delivery vehicles, such as liposomes. Alternatively, nucleic acids encoding the polypeptides can be administered as naked nucleic acids or in vectors, particularly gene therapy vectors. Such gene therapy can be effected ex vivo by removing cells from a subject, introducing the vector or nucleic acid into the cells and then reintroducing the modified cells. Gene therapy also can be effected in vivo by directly administering the nucleic acid or vector.

Treatments using the HGF isoforms provided herein, include, but are not limited to, treatment of diseases and conditions associated with cell proliferation and neovascularization including cancers and angiogenic diseases, including rheumatoid arthritis, diabetic retinopathy, and hemangiomas. Exemplary treatments and preclinical studies are described for treatments and therapies with HGF isoforms. Such descriptions are meant to be exemplary only and are not limited to a particular HGF isoform. One of skill in the art can determine the appropriate dosage of a molecule to administer based on the type of disease to be treated, the severity and course of the disease, whether the molecule is administered for preventive or therapeutic purposes, previous therapy, the patient's clinical history and response to therapy, and the discretion of the attending physician.

1. Cancer

HGF isoforms, including those provided herein, such as, but not limited to, the HGF isoforms (and encoding nucleic acids) set forth in SEQ ID NOS: 9-14 can be used in the treatment of cell proliferation diseases including cancers. HGF signaling contributes to cancer progression by affecting cellular processes such as cell growth, inhibition of apoptosis, cell morphogenesis, cell adhesion, and cell motility that are associated with tumor proliferation and invasion. Examples of cancers to be treated include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Cancers treatable with HGF isoforms are generally cancers expressing the MET receptor. Such cancers can be identified by any means known in the art for detecting MET expression, for example by RT-PCR or by immunohistochemistry.

Treatment of cancer with HGF isoforms can suppress tumor growth and metastases. For example, an animal model of tumor cell formation can be produced by injecting C6 glioma cells into immunocompromised athymic nude mice. Administration of HGF isoforms, for example once daily, to the immunocompromised mice can decrease tumor volume and decrease cellular proliferation at the tumor site. In another model termed the Lewis lung carcinoma model, whereby distant metastases flourish upon removal of the primary tumor, administration of HGF isoforms just before and just after resection of primary tumors resulting from inoculation with wild-type Lewis lung carcinoma cells results in a decrease in the number of lung surface metastases.

HGF isoforms can be used to treat cancers that exhibit neovascularization of solid tumors. Tumor angiogenesis is critical to the growth and metastasis of tumors. Highly vascular tumors have an increased risk of developing metastases. HGF isoforms can inhibit blood vessel growth by inhibiting the actions of pro-angiogenic factors, such as FGF and VEGF, in addition to HGF. Therapies for the treatment of cancers with HGF isoforms include administration of predefined doses of HGF isoforms over a period of time to control to the vascularization and growth of the tumor. Exemplary cancers in which HGF isoforms can be used to inhibit tumor angiogenesis include, but are not limited to, carcinomas of the breast colon, gallbladder, stomach, lung, ovary, pancreas, and prostate, lymphomas, and malignant melanomas.

2. Angiogenic Diseases

HGF isoforms, including those provided herein, such as but not limited to, the HGF isoforms (and encoding nucleic acids) set forth in SEQ ID NOS: 9-14 can be used the treatment of diseases associated with aberrant angiogenesis including rheumatoid arthritis, osteoarthritis, psoriasis, Osler-Webber syndrome, endometriosis, Still's disease, angiogenesis of the heart-muscle, peripheral hemangiectasis, hemophilic arthritis, age-related macular degeneration, retinopathy of prematurity, rejection to keratoplasty, systemic lupus erythematosus, atherosclerosis, neovascular glaucoma, choroidal neovascularization, retrolental fibroplasias, perosis, neurofibroma, hemangioma, acoustic neuroma, neurofibroma, trachoma, suppurative granuloma, and diabetes-related diseases, such as proliferative diabetic retinopathy and vascular diseases. Exemplary non-limiting angiogenic diseases contemplated as disease targets for treatment using HGF isoforms are described below.

a. Arthritis and Chronic Inflammatory Diseases

HGF isoforms including, but not limited to, HGF isoforms described herein such as polypeptides that contain sequences of amino acids set forth in any of SEQ ID NOS: 10, 12, 14, 18, or 20, can be used in the treatment of inflammatory diseases and conditions, including arthritis, inflammatory lung disease, Crohn's disease, and psoriasis. The inflammatory response is characterized by dilation and increased permeability of the vasculature and activation of endothelial cells, followed by angiogenic remodeling of capillaries and venules. Although stimulation of angiogenic factors can be part of the normal inflammatory response, chronic inflammation is often characterized by significant increases in capillary density and excessive dilation of blood vessels. The inflammatory tissue is often hypoxic, which causes the upregulation of pro-angiogenic factors, such as VEGF, FGF, and HGF. Suppression of angiogenesis can decrease the nutrient supply to inflamed tissues, block the entry of inflammatory cells into the tissue, and prevent the endothelial cell activation and secretion of cytokines and extracellular matrix proteinases.

In the synovial fluid of patients with rheumatoid arthritis and osteoarthritis, elevated levels of VEGF, FGF, and HGF and other pro-angiogenic factors can be found. In rheumatoid arthritis, the synovial pannus becomes hyperplastic and invades articular cartilage and adjacent bone. A vascular reorganization occurs that results in increased vascular density in the synovium to provide the necessary oxygen and nutrients to the invading pannus. The increased vascular permeability may also increase oedema and joint swelling. In osteoarthritis, vascular reorganization in the synovium also occurs; however, instead of degradation of the bone and cartilage by an invading pannus, chondrocyte hypertrophy and endochondral ossification occurs by direct vascular invasion of the cartilage and increased vascularization at the osteochondral junction. Treatment of rheumatoid arthritis and osteoarthritis with HGF isoforms, including one or more of the isoforms set forth as SEQ ID NOS: 10, 12, 14, 18, or 20, can ameliorate the symptoms associated with these diseases by inhibiting the neovascularization processes that lead to joint damage.

Chronic fibroproliferative disorders such as inflammatory pulmonary fibrosis exhibit dysregulated angiogenesis and may contribute to fibroplasia and deposition of extracellular matrix. Stimulation of angiogenesis occurs due to the imbalance of pro-angiogenic factors that are upregulated during lung inflammation. Extensive neovascularization is observed in the lungs of patients with widespread interstitial fibrosis. Vascular redistribution may also impair gas exchange through decreased vessel densities in the alveolar walls in favor of vessel formation near the inflamed tissue which diverts blood flow further away from needed airspaces. Treatment of pulmonary inflammation with HGF isoforms, including one or more of the isoforms set forth as SEQ ID NOS: 10, 12, 14, 18, or 20, can aid in preventing unwanted redistribution of the vascular network and decreasing tissue inflammation.

Vascular dilation and expansion also play a part in the progression of other inflammatory diseases such as psoriasis and Crohn's disease. Poriatic skin is characterized by abnormally proliferating epithelial cells and blood vessels, capillary vessel leakage and overproduction of pro-angiogeneic factors, including VEGF and IL-8. The vasculature beneath psoriatic lesions is abundant and elongated. Skin lesions that show increased expression of VEGF also display abundant VEGF receptor expression in the underlying endothelium. Similarly, increased levels of VEGF are observed in the serum of patients with inflammatory bowel diseases, such as Crohn's disease. Increased vascular permeability may contribute to recruitment of macrophage infiltration and stimulation of immune responses against the injured tissue. Treatment of inflammatory disorders, such as psorisis and Crohn's disease, with HGF isoforms, including one or more of the isoforms set forth as SEQ ID NOS: 10, 12, 14, 18, or 20, can ameliorate the symptoms associated chronic inflammation.

HGF isoforms also can be used to treat vascular diseases, such as atherosclerosis. Stimulation of angiogenesis can contribute to the formation and growth of atherosclerotic plaques through increased vascular dilation and recruitment of macrophages to the vessel lesions. Increased inflammatory responses at sites of atherosclerotic plaques leads to expansion of the lesion. Treatment with HGF isoforms, including one or more of the isoforms set forth as SEQ ID NOS: 10, 12, 14, 18, or 20, can be used to inhibit plaque growth in atherosclerotic disease.

b. Ocular Diseases

HGF isoforms including, but not limited to, HGF isoforms described herein such as polypeptides that contain sequences of amino acids set forth in any of SEQ ID NOS: 10, 12, 14, 18, or 20, can be used in the treatment of ocular diseases and conditions, including age-related macular degeneration and proliferative diabetic retinopathy. Age-related macular degeneration is associated with vision loss resulting from accumulated macular drusen, extracellular deposits in Brusch's membrane, and retinal pigment epithelium (RPE) dysfunction due to degenerative cellular and molecular changes in RPE and photoreceptors overlying the macular drusen. The cellular and molecular changes occurring in the RPE, in part due to oxidative stress in the aging eye, include altered expression of genes for cytokines, matrix organization, cell adhesion, and apoptosis resulting in the possible induction of a focal inflammatory response at the RPE-Bruch's membrane border. For example, oxidative stress induces the accumulation of angiogenic factors, including HGF, in the RPE and photoreceptor layers in early age-related macular degeneration, and induces a variety of inflammatory events including NFκB nuclear localization, and apoptosis. HGF stimulates the division and migration of RPE and blood vessel endothelial cells. HGF also stimulates the production of other growth factors that promote the formation of new blood vessels and supports neovascularization directly by invasion of the blood vessel cells into the extracellular matrix. Treatment of early stage age-related macular generation with HGF isoforms, including one or more of the isoforms set forth as SEQ ID NOS: 10, 12, 14, 18, or 20, can ameliorate one or more symptoms of the disease.

Proliferative diabetic retinopathy (PDR) is characterized by neovascularization of the retina and intrusion of blood vessels into the vitreous cavity, that leads to bleeding and scarring around proliferative channels. HGF expression is significantly elevated in the vitreous fluid of the eyes of patients with PDR. VEGF expression is also upregulated in PDR and may be important in the early stages to increase vascular permeability, while HGF functions at a later stage in growth and activation of endothelial cells needed for neovascularization. Treatment of PDR with HGF isoforms, including one or more of the isoforms set forth as SEQ ID NOS: 10, 12, 14, 18, or 20, can aid in the inhibition of retinal vessel growth stimulated by HGF and VEGF pathways.

c. Endometriosis

HGF isoforms including, but not limited to, HGF isoforms described herein such as polypeptides that contain sequences of amino acids set forth in any of SEQ ID NOS: 10, 12, 14, 18, or 20, can be used in the treatment of endometriosis. Regulated angiogenesis is a normal process that occurs during the female menstrual cycle; however, in endometriosis, the endometrium exhibits excessive angiogenesis, characterized by enhanced endothelial cell proliferation. These endothelial cells have a high expression of the pro-angiogenic αᵥβ₃ integrin. The increased growth mimics some of the characteristics of tumor growth by the formation of nodules or lesions that implant and grow in areas of the peritoneal cavity including the ovaries, fallopian tubes, the ligaments supporting the uterus, the area between the vagina and the rectum, the outer surface of the uterus, and the lining of the pelvic cavity. Growths can also be found in abdominal surgery scars, on the intestines or in the rectum, on the bladder, vagina, cervix, and vulva. Treatment of endometriosis with HGF isoforms, including one or more of the isoforms set forth as SEQ ID NOS: 10, 12, 14, 18, or 20, can aid in the inhibition of excessive endometrial vessel formation and nodule growth.

3. Malaria

HGF isoforms, including, but not limited to, HGF isoforms described herein such as polypeptides that contain sequences of amino acids set forth in any of SEQ ID NOS: 10, 12, 14, 18, or 20, can be used in the treatment of malaria. The causative agent of malaria is Plasmodium which infects hepatocytes to initiate mammalian infection. HGF renders hepatocytes susceptible to infection which is dependent upon signaling of HGF through its receptor MET. MET signaling induced by HGF induces morphogenic rearrangements of the host-cell cytoskeleton that are required for the early development of the parasites within hepatocytes. Infection of hepatocytes by Plasmodium also is contributed to by anti-apoptotic signals induced by HGF-MET signaling. Treatment of malaria with HGF isoforms, including one or more of the isoforms set forth as SEQ ID NOS: 10, 12, 14, 18, or 20 can prevent malaria infection.

4. Combination Therapies

HGF isoforms, including those provided herein, such as but not limited to, the HGF isoforms (and encoding nucleic acids) set forth in SEQ ID NOS: 10, 12, 14, 18, or 20, can be used in combination with each other, and/or in combination with other agents, molecules, and or existing drugs and therapeutics to treat diseases and conditions, particularly those involving cancers and other proliferative disorders and/or aberrant angiogenesis as set forth herein and known to those of skill in the art. For example, an HGF isoform can be administered with an anti-tumor agent that treats cancers including squamous cell cancer (e.g. epithelial squamous cell cancer), lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer, and other cancers where aberrant MET activation is involved.

Examples of anti-tumor agents include angiogenesis inhibitors, anti-proliferative agents, bone resorption inhibitors, DNA modification/repair agents, DNA synthesis inhibitors, DNA-RNA transcription regulators, enzyme activators, enzyme inhibitors, HSP-90 inhibitors, microtubule inhibitors, and other therapy adjuncts. Exemplary anti-tumor agents that can be used in combination with HGF isoforms include, but are not limited to, angiostatin, DL-α-difluoromethylomithine hydrochloride solid, endostatin, genistein, staurosporine, thalidomide, N-acetyl-D-sphingosine, aloe-emodine, apigenin, berberine chloride form, dichloromethylenediphosphonic acid disodium salt, emodin, N-hexanoyl-D-sphingosine, 7β-hydroxycholesterol, 25-hydroxycholesterol, hyperforin, parthenolide, rapamycin, alendronate sodium trihydrate, etidronate disodium solid, pamidronate disodium salt, aphidicolin, bleomycin sulfate, carboplatin, carmustine, chlorambucil, cyclophosphamide monohydrate, dacarbazine, cis-diammineplatimun(II)dichloride crystalline, 6,7-dihydroxycourmain, melphalan powder, methoxyamine hydrochloride, mitomycin C, mitoxantrone dihydrochloride, oxaliplatin solid, amethopterin, cytosine β-D-arabinofuranoside, 5-fluoro-5′-deoxyuridine, ganciclovir, hydroxyurea, 6-mecaptopurine monohydrate, Daunorubicin hydrochloride, (−)-Deguelin, formestane, Fostriecin, indomethacin, oxamflatin, tryphostin AG, urinary trypsin inhibitor, cholecalciferol, melatonin, raloxifene hydrochloride, tamoxifen, troglitazone, and/or geldanamycin.

An HGF isoform can be administered in combination with other agents that inhibit MET activation. For example, an HGF isoform can be administered with other antagonist or neutralizing agents of a MET receptor such as for example an anti-HGF antibody, an uncleavable pro-HGF, a recombinant Sema domain of MET, and/or a soluble MET isoform. Exemplary soluble MET isoforms can include any one of the MET isoforms set forth in SEQ ID NOS:84-114. An HGF isoform also can be administered in combination with agents that prevent MET dimerization and signaling such as a dominant-negative receptor, anti-MET Sema antibodies, ATP competitors, SH2 competitors, inhibitors of specific transducers such as for example PtdIns3K, MAPK, or STAT3 inhibitors, and/or antisense, ribozyme, RNAi or other molecules that silence MET expression.

Combinations of HGF isoforms with intron fusion proteins and other agents, including cell surface receptor (CSR) polypeptide isoforms for treating cancers and other disorders involving aberrant angiogenesis are contemplated (see, e.g. those described herein and in copending and published applications U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application No. WO 00/44403, WO 01/61356, WO 2005/016966). The cell surface receptor isoforms can include MET isoforms or other cell surface receptor isoforms including isoforms of receptor tyrosine kinases or tumor necrosis factor receptors, such as members of the VEGFR, FGFR, PDGFR, MET, TIE, Eph, RAGE, and TNFR families. These can include isoforms of CSRs including ErbB2 (HER2), ErbB3, ErbB4, DDR1, DDR2, EphA1, EphA2, EphA3, EphA4, EphA5, EphA6, EphA7, EphA8, EphB1, EphB2, EphB3, EphB4, EphB5, EphB6, FGFR-1, FGFR-2, FGFR-3, FGFR-4, PDGFR-B, TEK, Tie-1, KIT, VEGFR-1, VEGFR-2, VEGFR-3, Flt1, Flt3, TNFR1, TNFR2, RON, CSF1R, and RAGE. Exemplary of such isoforms are the herstatins (see, SEQ ID NOS: 186-200), and polypeptides that include the intron portion of a herstatin (see, SEQ ID NOS: 216-230), as well as isoforms and encoding nucleotide sequences set forth in any of SEQ ID NOS:36-185. The combinations of isoforms and/or drug agent and HGF isoform selected is a function of the disease to be treated and is based upon consideration of the target tissues and cells and receptors expressed thereon.

The combinations can target two or more cell surface receptors or steps involved in cancer cell proliferation, growth, invasion, and metastasis, and/or steps involved in angiogenic and/or endothelial cell maintenance pathways or can target two or more cell surface receptors or steps in a disease process, such as any in which one or both of these pathways are implicated, such as angiogenic diseases including diabetes, cancers and all other noted herein and known to those of skill in the art. The two or more agents can be administered as a single composition or can be administered as two or more compositions (where there are more than two agents) simultaneously, intermittently or sequentially. They can be packaged as a kit that contains two or more compositions separately or as a combined composition and optionally with instructions for administration and/or devices for administration, such as syringes.

5. Evaluation of HGF Isoform Activities

If needed animal models can be used to evaluate HGF isoforms that are candidate therapeutics. Parameters that can be assessed include, but are not limited to, efficacy and concentration-response, safety, pharmacokinetics, interspecies scaling and tissue distribution. Model animal studies include assays such as described herein as well as those known to one of skill in the art. Animal models can be used to obtain data that then can be extrapolated to human dosages for design of clinical trials and treatments with HGF isoforms, for example, efficacy and concentration-response can be extrapolated from animal model results.

### O. EXAMPLE

The following example is included for illustrative purposes only and are not intended to limit the scope of the invention.

### Example

### Cloning HGF Isoforms

A. Preparation of Messenger RNA

mRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

mRNA was denatured at 70° C. in the presence of 40% DMSO for 10 min and quenched on ice. First-strand cDNA was synthesized with either 200 ng oligo(dT) or 20 ng random hexamers in a 20 μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of Stratascript reverse transcriptase (Stratagene, La Jolla, Calif.). After incubation at 37° C. for 1 h, the cDNA from both reactions were pooled and treated with 10 units of RNase H (Promega, Madison, Wis.).

C. PCR Amplification

Forward and reverse primers for RT-PCR cloning were designed to clone splice variants of HGF. Forward primers (F1, F2) were selected flanking the start codon and reverse primers (intron11R1, intron11R2, or intron13R1) were selected from the intron sequence of the HGF genomic sequence (Table 6) using the method described by Hiller et al (Genome Biology 2005. 6: R58)(see Table 7). Each PCR reaction contained 10 ng of reverse-transcribed cDNA, 0.2 μM F1/R1 primer mix, 1 mM Mg(OAc)₂, 0.2 mM dNTP (Amersham, Piscataway, N.J.), 1×XL-Buffer, and 0.04 U/μl rTth DNA polymerase (Applied Biosystems) in a total volume of 70 μl. PCR conditions were 36 cycles of 94° C. for 45 sec, 60° C. for 1 min, and 68° C. for 2 min. The reaction was terminated with an elongation step of 68° C. for 20 min. Nested PCR was performed with 1 μp of RT-PCR product from above, F2/R2 primer mix, 1 mM Mg(OAc)₂, 0.2 mM dNTP, 1×XL-Buffer, and 0.04 U/μl rTth DNA polymerase (Applied Biosystems) in a total volume of 70 ul. PCR conditions were 33 cycles of 94° C. for 45 sec, 60° C. for 1 min, and 68° C. for 2 min. The reaction was terminated with an elongation step of 68° C. for 20 min.

TABLE 6
NUCLEIC ACID FOR CLONING HGF ISOFORMS

| | Genomic | | nt | | SEQ | | amino acid | SEQ |
|---|---|---|---|---|---|---|---|---|
| Member | SEQ ID NO: | nt ACC. # | length | CDS | ID NO: | prt ACC. # | length | ID NO: |
| HGF | 1 | NM_000601 | 2820 | 166-2352 | 2 | NP_000592 | 728 | 3 |

TABLE 7

PRIMERS FOR PCR CLONING

SEQ
ID

| | | |
|---|---|---|
| NO | Primer | Sequence |

| | | |
|---|---|---|
| 4 | HGF_F1 | AGG ATT CTT TCA CCC AGG CA |
| 5 | HGF_intron11R1 | GAA TAA ATG CCA GAC CAC CTA |
| 6 | HGF_F2 | ACC ATG TGG GTG ACC AAA CT |
| 7 | HGF_intron11R2 | TCA CAA GAC ACC AAT CCC TAA CT |
| 8 | HGF_intron13R1 | TCC ATA TTT CTG GGA ATA GGA GGA |
| | | C |

D. Cloning and Sequencing of PCR Products

PCR products were electrophoresed on a 0.8% agarose gel, and DNA from detectable bands were stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into DH10B cells (Invitrogen, Carlsbad, Calif.). Recombinant plasmids were selected on LB agar plates containing 25 μg/ml kanamycin, 0.1 mM IPTG, and 60 μg/ml X-gal. For each transfection, 12 colonies were randomly picked and their cDNA insert sizes were determined by PCR with UA vector primers. Clones were then sequenced from both directions with M13 forward and reverse vector primers. All clones were sequenced entirely using custom primers for directed sequencing completion across gapped regions.

E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding HGF isoforms were studied further (see below, Table 8).

F. Exemplary HGF Isoforms

Exemplary nucleic acid molecules encoding HGF isoforms, prepared using the methods described herein, are set forth below in Table 8. Nucleic acid molecules encoding HGF isoforms are provided and sequences thereof are set forth in SEQ ID NOS: 9, 11, 13, 17, or 19. The sequence of polypeptides of exemplary HGF isoforms are set forth in SEQ ID NOS: 10, 12, 14, 18, or 20.

TABLE 8
Nucleic acid molecules encoding HGF Isoforms

| | | | Amino Acid | SEQ ID |
|---|---|---|---|---|
| Gene | ID | Type | Length | NOS |
| HGF | SR023A02 | Intron fusion | 467 | 10, 18 |
| HGF | SR023A08 | Intron fusion | 472 | 12, 20 |
| HGF | SR023E09 | Intron fusion | 514 | 14 |

Since modifications will be apparent to those of skill in this art, it is intended that this invention be limited only by the scope of the appended claims.

1. An isolated HGF polypeptide isoform, comprising all or a portion of a K4 domain of an HGF ligand, wherein the HGF polypeptide isoform is an intron fusion protein.

2. The isolated HGF polypeptide isoform of claim 1, wherein the HGF polypeptide is encoded by a sequence of nucleotides that includes all or a portion of an intron selected from among introns 1, 2, 3, 4, 5, 6, 7, 8, 9, 10, 11, 12, 13, 14, 15, 16, and 17 of a cognate HGF gene.

3. The isolated HGF polypeptide isoform of claim 1, wherein the sequence of the cognate HGF gene is set forth in SEQ ID NO:1, or is an allelic or species variant thereof.

4. The isolated HGF polypeptide of claim 3, wherein the HGF polypeptide has at least 80%, 85%, 90%, 95%, 96%, 97%, 98% or 99% sequence identity along its full length with a sequence of amino acids encoded by the corresponding portions of SEQ ID NO: 1.

5. The isolated HGF polypeptide of claim 3, wherein the cognate HGF polypeptide has at least 80%, 85%, 90%, 95%, 96%, 97%, 98% or 99%. sequence identity with the sequence of amino acids encoded by SEQ ID NO: 1.

6. The isolated HGF polypeptide isoform of claim 1, further comprising all or part of a N-terminal domain, all or part of a K1 domain, all or part of a K2 domain, or all or part of a K3 domain or combinations thereof.

7. The isolated HGF polypeptide isoform of claim 2, wherein the intron is all or a portion of intron 11.

8. The isolated HGF polypeptide isoform of claim 1, wherein the polypeptide is operatively linked to at least one amino acid encoded by intron 11.

9. The isolated HGF polypeptide isoform of claim 8, wherein the polypeptide comprises three amino acids encoded by intron 11.

10. The isolated HGF polypeptide isoform of claim 9, wherein the HGF polypeptide has at least 80%, 85%, 90%, 95%, 96%, 97%, 98% or 99% sequence identity with a sequence of amino acids set forth in any of SEQ ID NOS: 10, 12, 18, or 20.

11. The isolated HGF polypeptide isoform of claim 9 that comprises the sequence of amino acid set forth in any of SEQ ID NOS: 10, 12, 18, or 20, or is an allelic variant thereof.

12. The isolated HGF polypeptide isoform of claim 11, wherein the allelic variant comprises one or more amino acids of the allelic variations as set forth in SEQ ID NO: 16.

13. The isolated HGF polypeptide isoform of claim 1, further comprising all or part of a SerP domain.

14. The isolated HGF polypeptide of claim 13, wherein the intron is all or part of intron 13.

15. The isolated HGF polypeptide isoform of claim 14, wherein the HGF polypeptide has at least 80%, 85%, 90%, 95%, 96%, 97%, 98% or 99% sequence identity with a sequence of amino acids set forth in SEQ ID NO: 14.

16. The isolated HGF polypeptide isoform of claim 14 that comprises the sequence of amino acid set forth in SEQ ID NO: 14, or is an allelic or species variant thereof.

17. The isolated HGF polypeptide isoform of claim 16, wherein the variant comprises one or more amino acids of the allelic variations as set forth in SEQ ID NO: 16.

18. The isolated HGF polypeptide isoform of claim 15, wherein the polypeptide contains the same number of amino acids as the polypeptide set forth in SEQ ID NO: 14.

19. The isolated HGF polypeptide isoform of claim 1 that is an antagonist of a cognate HGF polypeptide.

20. The isolated HGF polypeptide isoform of claim 19, wherein the polypeptide binds to a MET receptor.

21. The isolated HGF polypeptide isoform of claim 20, wherein the polypeptide inhibits a MET-mediated activity selected from among one or more of mitogenesis, morphogenesis, and motogenesis.

22. The isolated HGF polypeptide isoform of claim 1 that inhibits angiogenesis.

23. The isolated HGF polypeptide isoform claim 22, wherein the polypeptide binds to a glycosaminoglycan.

24. The isolated HGF polypeptide isoform of claim 23, wherein the glycosaminoglycan is heparin sulfate.

25. The isolated HGF polypeptide isoform of claim 22, wherein the polypeptide binds to an angiogenic molecule.

26. The isolated HGF polypeptide isoform of claim 25, wherein the angiogenic molecule is selected from among ATP synthase, angiomotin, αvβ3 integrin, annexin II, MET, VEGFR, and FGFR.

27. The isolated HGF polypeptide isoform of claim 22 that inhibits angiogenesis induced by a cognate HGF, FGF-2, or VEGF.

28. The isolated HGF polypeptide isoform of claim 1 that is an HGF antagonistic and inhibits angiogenesis.

29. A pharmaceutical composition, comprising an HGF polypeptide isoform of claim 1.

30. The composition of claim 29, comprising an amount of the polypeptide effective for antagonizing a cognate HGF polypeptide.

31. The composition of claim 30, wherein antagonizing a cognate HGF inhibits one or more of a MET-mediated activity selected from among any one or more of mitogenesis, motogenesis and morphogenesis.

32. The composition of claim 29, comprising an amount of the polypeptide effective for inhibiting angiogenesis.

33. The composition of claim 32, wherein the polypeptide inhibits angiogenesis induced by a cognate HGF, FGF-2, or VEGF.

34. The composition of claim 29, further comprising an anti-cancer agent and/or an anti-angiogenesis agent.

35. A nucleic acid molecule encoding an HGF polypeptide of claim 1.

36. A nucleic acid molecule, comprising at least all or part of one intron and an exon of an HGF gene, but not containing intron 5.

37. A nucleic acid molecule of claim 36, wherein:
the intron contained in the molecule contains a stop codon;
the nucleic acid molecule encodes an open reading frame that spans an exon intron junction; and
the open reading frame terminates at the stop codon in the intron.

38. The nucleic acid molecule of claim 37, wherein the intron encodes one or more amino acids of the encoded polypeptide.

39. The nucleic acid molecule of claim 38, wherein the intron is all or a portion of intron 11.

40. The nucleic acid molecule of claim 39, comprising a sequence of nucleotides set forth in any one of SEQ ID NOS: 9, 11, 17 and 19, or an allelic or species variant thereof.

41. The nucleic acid molecule of claim 40, wherein the allelic variant is any one of the allelic variations set forth in SEQ ID NO:15.

42. The nucleic acid molecule of claim 37, wherein the stop codon is the first codon in the intron.

43. The nucleic acid molecule of claim 42, wherein the intron is all or a portion of intron 13.

44. The nucleic acid molecule of claim 43, comprising a sequence of nucleotides set forth in SEQ ID NO: 13 or an allelic variant thereof.

45. The nucleic acid molecule of claim 44, wherein the allelic variant is any one of the allelic variations set forth in SEQ ID NO:15.

46. A nucleic acid molecule, wherein the nucleic acid molecule is selected from among:
a) a nucleic acid molecule comprising a sequence of nucleotides set forth in any of SEQ ID NOS: 9, 11, 13, 17, 19, and allelic variants or species thereof;
b) a nucleic acid molecule that encodes a polypeptide of claim 1 and has at least 60%, 65%, 70%, 75%, 80%, 85%, 90%, 91%, 92%, 93%, 94%, 95%, 96%, 97%, 98% or 99% sequence identity to any of SEQ ID NOS: 9, 11, 13, 17, or 19;
c) a nucleic acid that hybridizes under conditions of medium or high stringency along at least 70% of its full length to a nucleic acid molecule comprising a sequence of nucleotides set forth in any of SEQ ID NOS: 9, 11, 13, 17, or 19 wherein the encoded polypeptide contains a K4 domain and contains at least one codon from an intron;
d) a nucleic acid molecule that comprises degenerate codons of a), b), or c); and
e) a nucleic acid molecule that is a splice variant of an HGF gene wherein the nucleic acid molecule includes all or a portion of an intron other than intron 5.

47. A polypeptide encoded by a nucleic acid molecule of claim 35.

48. A vector, comprising the nucleic acid molecule of claim 35.

49. The vector of claim 48 that is a mammalian expression vector.

50. The vector of claim 48 that is selected from among an adenovirus vector, an adeno-associated virus vector, EBV, SV40, a cytomegalovirus vector, a vaccinia virus vector, a herpesvirus vector, a retrovirus vector, a lentivirus vector and an artificial chromosome.

51. The vector of claim 50 that is episomal or that integrates into a chromosome of a cell into which it is introduced.

52. A cell, comprising the vector of claim 48.

53. A method of treatment of an HGF-mediate disease, comprising administering to a subject a nucleic acid molecule of claim 35.

54. The method of treatment of claim 53, wherein the nucleic acid molecule is introduced into a vector for administration.

55. The method of treatment of claim 54, wherein the vector is an expression vector.

56. The method of treatment of claim 55, wherein the vector is episomal.

57. The method of treatment of claim 55, wherein the expression vector is selected from among an adenovirus vector, an adeno-associated virus vector, EBV, SV40, a cytomegalovirus vector, a vaccinia virus vector, a herpesvirus vector, a retrovirus vector, a lentivirus vector, or an artificial chromosome.

58. The method of treatment of claim 53, wherein the nucleic acid is administered in vivo or ex vivo.

59. The method of treatment of claim 58, wherein ex vivo treatment comprises administering the nucleic acid into a cell in vitro, followed by administration of the cell into the subject.

60. The method of treatment of claim 59, wherein the cell is from a suitable donor or from the subject to be treated.

61. The method of treatment of claim 53, wherein the subject is a human.

62. A pharmaceutical composition, comprising a nucleic acid molecule of claim 35.

63. A method of treating an HGF-mediate disease or condition, comprising, administering a pharmaceutical composition of claim 29.

64. The method of claim 63, wherein the pharmaceutical composition contains a polypeptide that inhibits angiogenesis, cell proliferation, cell migration, tumor cell growth or tumor cell metastasis.

65. The method of claim 63, wherein the disease or condition is selected from the group consisting of cancer, angiogenic disease, or malaria.

66. The method of claim 65, wherein the angiogenic disease is selected from among ocular disease, endometriosis, arthritis, or other chronic inflammatory diseases.

67. The method of claim 66, wherein the angiogenic disease is selected from among rheumatoid arthritis, osteoarthritis, psoriasis, Osler-Webber syndrome, endometriosis, Still's disease, angiogenesis of the heart-muscle, peripheral hemangiectasis, hemophilic arthritis, age-related macular degeneration, retinopathy of prematurity, rejection to keratoplasty, systemic lupus erythematosus, atherosclerosis, neovascular glaucoma, choroidal neovascularization, retrolental fibroplasias, perosis, neurofibroma, hemangioma, acoustic neuroma, neurofibroma, trachoma, suppurative granuloma, and diabetes-related diseases, such as proliferative diabetic retinopathy and vascular diseases, inflammatory lung disease, Crohn's disease, and psoriasis.

68. The method of claim 66, wherein the cancer is selected from the group consisting of carcinoma, lymphoma, blastoma, sarcoma, and leukemia or lymphoid malignancies, squamous cell cancer, lung cancer, small-cell lung cancer, non-small-cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

69. A conjugate, comprising an HGF isoform of claim 1.

70. The conjugate of claim 69, wherein:
the conjugate comprises an HGF isoform or domain thereof or functional portion thereof, and a second portion from a different HGF isoform or from another cell surface receptor (CSR) isoform; and
the portions are linked directly or via a linker.

71. The conjugate of claim 70, wherein the second portion from a cell surface receptor isoform is all or part of an extracellular domain of the cell surface receptor isoform.

72. The conjugate of claim 70, wherein the cell surface receptor isoform is a receptor tyrosine kinase.

73. The conjugate of claim 70, wherein the second portion is all or part of a herstatin polypeptide.

74. The conjugate of claim 73, wherein the herstatin polypeptide comprises a sequence of amino acids set forth in any one of SEQ ID NOS:186-200.

75. A chimeric polypeptide, comprising:
all of or at least one domain of an HGF isoform of claim 1; and
all of or at least one domain of a different HGF isoform or of another cell surface receptor isoform.

76. The polypeptide of claim 75, wherein the cell surface receptor isoform is an intron fusion protein.

77. The polypeptide of claim 76, comprising all of or at least one domain of an HGF isoform and an intron-encoded portion of a cell surface receptor isoform.

78. A combination, comprising:
one or more HGF isoform(s) of claim 1;
one or more other cell surface receptor isoforms; and/or
a therapeutic drug.

79. The combination of claim 78, wherein the isoforms and/or drugs are in separate compositions or in a single composition.

80. The combination of claim 78, wherein the cell surface receptor isoform is an isoform of a VEGFR, FGFR, DDR, TNFR, PDGFR, MET, TIE, RAGE, EPH, or HER.

81. The combination of claim 80, wherein the cell surface receptor isoform is a MET isoform.

82. The combination of claim 78, wherein the isoform is an intron fusion protein.

83. The combination of claim 81, wherein the MET isoform comprises a sequence of amino acids selected from any one of SEQ ID NOS: 85, 87, 89, 91, 93, 95, 97, 99, 101, 103, 105, 107, 109, 111, 113, and 114.

84. A method of treatment of an HGF-mediated disease, comprising administering the components of the combination of claim 78, wherein each component is administered separately, simultaneously, intermittently, in a single composition or combinations thereof.

85. A method of inhibiting tumor invasion or metastasis of a tumor, comprising administering a composition of claim 29.

86. A method of inhibiting angiogenesis, comprising administering a composition of claim 29.

87. A fusion protein or conjugate, comprising a fragment of a CD45 polypeptide linked directly or via a linker to a protein, wherein;
the fragment of CD45 is selected to add carbohydrates or glycosylation sites.

88. The fusion protein of claim 87, wherein the protein is a therapeutic protein.

89. The fusion protein of claim 87, wherein the fusion protein is a cell surface receptor (CSR) or ligand isoform or is a cytokine or CSR or ligand or growth factor or hormone or forms thereof that include additional amino acids on the end.

90. The fusion protein of claim 87, wherein the CD45 polypeptide comprises a sufficient number of glycosylation sites or carbohydrates, whereby serum half-life of the protein is increased by 1%, 5%, 10%, 20%, 30%, 40%, 50%, 60%, 70%, 80%, 90%, 95%, 96%, 97%, 98%, 99% or more.

91. The fusion protein or conjugate of claim 87, wherein the linkage is a chemical linkage optionally including a chemical linker.

92. The fusion protein or conjugate of claim 91, wherein the linker is produced from a heterobifunctional linker and/or is a photocleavable linker.

93. The fusion protein or conjugate of claim 87 that is a fusion protein that optionally also includes a polypeptide or peptide or amino acid linker.

94. The fusion protein or conjugate of claim 93, wherein the linker contains 1-30, 1-10, 2-10 or 2-15 amino acid residues.

95. The fusion protein or conjugate of claim 87, wherein the CD45 polypeptide or fragment thereof comprises the sequence of amino acids set forth in any of SEQ ID NOS: 272, 274, 275, 276, 277, 278, 279, 281, 283, 285, 287, 289, 291, 293, and 295, and fragments thereof and variants thereof.

96. The fusion protein or conjugate of claim 87, wherein the protein is a ligand or CSR isoform or a form thereof containing additional amino acids.

97. The fusion protein or conjugate of claim 96, wherein the protein comprises a sequence of amino acids set forth in any of SEQ ID NOS: 3, 10, 12, 14, 16, 18, 20, 22, 24, 26, 28, 30, 32, 34, 35, 37, 39, 40, 42, 44, 46, 47, 49, 50, 52, 54, 56, 58, 59, 60, 61, 62, 63, 64, 65, 67, 69, 71, 73, 75, 77, 78, 80, 82, 83, 85, 87, 89, 91, 93, 95, 97, 99, 101, 103, 105, 107, 109, 111, 113, 114, 116, 118, 120, 122, 124, 126, 127, 128, 130, 132, 134, 136, 138, 140, 142, 144, 145, 146, 147, 148, 150, 152, 154, 156, 158, 160, 161, 162, 163, 164, 165, 166, 167, 169, 171, 172, 173, 174, 175, 176, 177, 179, 181, 183, 185, 186, 187, 188, 189, 190, 191, 192, 193, 194, 195, 196, 197, 198, 199, 200, 216, 217, 218, 219, 220, 221, 222, 223, 224, 225, 226, 227, 228, 229, 230, 246, 247, 248, 249, 250, 251, and allelic variants thereof.

98. The fusion protein or conjugate of claim 97, wherein the protein is HGF or an isoform thereof.

99. A kit, comprising:
a combination of claim 78; and optionally
one or more of instructions for use of the combination and instructions for use thereof.

100. The isolated HGF polypeptide isoform of claim 2, further comprising all or part of a SerP domain.

101. A polypeptide encoded by a nucleic acid molecule of claim 36.

102. A polypeptide encoded by a nucleic acid molecule of claim 46.

103. A pharmaceutical composition, comprising a vector of claim 48.

104. A method of treating an HGF-mediate disease or condition, comprising, administering a pharmaceutical composition of claim 62.

105. The method of claim 104, that results in inhibition of tumor invasion or metastasis of a tumor or angiogenesis.

106. The isolated HGF polypeptide isoform of claim 9, wherein the polypeptide contains the same number of amino acids as set forth in any of SEQ ID NOS: 10, 12, 18, or 20.
